(19) United States
(12) Patent Application Publication
(10) Pub. No.: US 2010/0009957 A1
(43) Pub. Date: Jan. 14, 2010

(54) NOVEL INHIBITORS OF BETA-LACTAMASE

(76) Inventors: Timothy A. Blizzard, Princeton, NJ (US); Helen Y. Chen, Marlboro, NJ (US); Jane Yang Wu, Marlboro, NJ (US); Seongkon Kim, Holmdel, NJ (US); Sookhee Ha, Warren, NJ (US); Christopher J. Mortko, Hoboken, NJ (US); Narayan Variankaval, Plainsboro, NJ (US); Anna Chiu, Redwood City, CA (US)

Correspondence Address: MERCK AND CO., INC, P O BOX 2000, RAHWAY, NJ, 07065-0907, US

(21) Appl. No.: 12/442,816

(22) PCT Filed: Sep. 24, 2007
(86) PCT No.: PCT/US07/20608

§ 371 Date: Mar. 25, 2009

Related U.S. Application Data
(60) Provisional application No. 60/847,453, filed on Sep. 27, 2006.

(51) Int. Cl. A61K 31/397; C07D 205/12; A61P 31/04

(52) U.S. Cl. 514/210.05; 540/203

(57) ABSTRACT

A class of 7-oxo-2,6-diazabicyclo-[3.2.0]-heptane-6-sulfonic acid compounds substituted at the two position of the bicyclic ring with a heterocyclylaminocarbonyl group or a carbocyclylaminocarbonyl group are β-lactamase inhibitors. The compounds and their prodrugs and pharmaceutically acceptable salts are useful in the treatment of bacterial infections in combination with β-lactam antibiotics. In particular, the compounds are suitable for use with β-lactam antibiotics (e.g., imipenem and ceftazidime) against micro-organisms resistant to β-lactam antibiotics due to the presence of the β-lactamases.

This application claims the benefit of U.S. Provisional Application No. 60/847,453 (filed Sep. 27, 2006), the disclosure of which is hereby incorporated by reference in its entirety.

### FIELD OF THE INVENTION

This invention relates to novel beta-lactamase inhibitors and their use against bacterial antibiotic resistance. More particularly, the invention relates to compositions and methods for overcoming bacterial antibiotic resistance.

### BACKGROUND OF THE INVENTION

Bacterial antibiotic resistance has become one of the most important threats to modern health care. Cohen, Science 1992, 257: 1051-1055 discloses that infections caused by resistant bacteria frequently result in longer hospital stays, higher mortality and increased cost of treatment. Neu, Science 1992, 257: 1064-1073 discloses that the need for new antibiotics will continue to escalate because bacteria have a remarkable ability to develop resistance to new agents rendering them quickly ineffective.

The present crisis has prompted various efforts to elucidate the mechanisms responsible for bacterial resistance, Coulton et al., Progress in Medicinal Chemistry 1994, 31: 297-349 teaches that the widespread use of penicillins and cephalosporins has resulted in the emergence of β lactamases, a family of bacterial enzymes that catalyze the hydrolysis of the β-lactam ring common to numerous presently used antibiotics. More recently, Dudley, Pharmacotherapy 1995, 15: 9S-14S has disclosed that resistance mediated by β-lactamases is a critical aspect at the core of the development of bacterial antibiotic resistance. Clavulanic acid, which is a metabolite of Streptomyces clavuligerus, and two semi-synthetic inhibitors, sulbactam and tazobactam are presently available semi-synthetic or natural product β-lactamase inhibitors. U.S. Pat. No. 5,698,577, U.S. Pat. No. 5,510,343, U.S. Pat. No. 6,472,406 and Hubschwerlen et al., J. Med. Chem. 1998, 41: 3961 and Livermore et al., J. Med. Chem. 1997, 40: 335-343, disclose certain synthetic β-lactamase inhibitors.

The availability of only a few β-lactamase inhibitors, however, is insufficient to counter the constantly increasing diversity of β-lactamases, for which a variety of novel and distinct inhibitors has become a necessity. There is, therefore, a need for new β-lactamase inhibitors.

### SUMMARY OF THE INVENTION

This invention provides novel substituted bicyclic beta-lactams which are surprisingly potent beta-lactamase inhibitors and are useful in combination with a beta-lactam antibiotic for the treatment of antibiotic-resistant bacterial infections. The compounds inhibit β-lactamases and synergize the antibacterial effects of β-lactam antibiotics (e.g., imipenem and ceftazidime) against those micro-organisms normally resistant to the β-lactam antibiotics as a result of the presence of the β-lactamases. The compounds of the present invention are effective against class C β-lactamases and the combination of these beta lactamase inhibitors with a beta lactam antibiotic (e.g., imipenem) will enable treatment of bacterial infections caused by class C β-lactamase producing organisms. Thus, this invention also relates to the combination of the claimed compounds with relevant β-lactam antibiotics to extend the spectrum of antimicrobial activity of the antibiotic against class C β-lactamase producing bacteria such as Pseudomonas spp. The invention further relates to compositions containing compounds of this invention and a pharmaceutically acceptable carrier or carriers. It also relates to methods for treating bacterial infections and inhibiting bacterial growth using the compounds or compositions of this invention. Another aspect of this invention is the use of the claimed compounds in the manufacture of a medicament for treating bacterial infections. This and other aspects of the invention are realized upon consideration of the specification in its entirety.

### BRIEF DESCRIPTION OF THE DRAWINGS

FIG. 1 is the X-ray powder diffraction pattern for the crystalline dihydrate in Example 34.

FIG. 2 is the DSC curve for the crystalline dihydrate in Example 34.

FIG. 3 is the TGA cruve for the crystalline dihydrate in Example 34.

### DETAILED DESCRIPTION OF THE INVENTION

An embodiment of the present invention (alternatively referred to herein as “Embodiment E1”) is directed to novel compounds of Formula I:

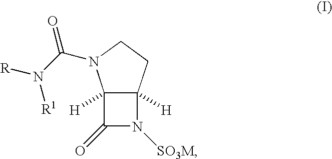

or a pro-drug or pharmaceutically acceptable salt thereof; wherein:
R represents a seven, eight, or nine membered saturated or unsaturated ring optionally containing one to three nitrogen, oxygen, or sulfur atoms and optionally substituted with one or more R^{a} groups;
R¹ represents hydrogen or C₁₋₃ alkyl;
R^{a} independently represents hydrogen, C₁₋₆ alkyl, substituted C₁₋₆ alkyl, C₁₋₆ alkenyl, substituted C₁₋₆ alkenyl, C₁₋₆ alkynyl, substituted C₁₋₆ alkynyl, halo; —CN; —NO₂; —OR^{b}, —SR^{b}; —N(R^{b})₂; —C(O)N(R^{b})₂; —SO₂N(R^{b})₂; —CO₂R^{b}; —C(O)R^{b}; —OCOR^{b}; —NHCOR^{b}; —NHC(O)₂R^{b}; —NHSO₂R^{b}; —C(NH)NH₂; —C(NH)H; or two R^{a} groups on the same ring carbon atom may be taken together with the carbon to represent a carbonyl group; or two or four R^{a} groups on the same ring sulfur atom may be taken together with the sulfur to represent a sulfinyl group or a sulfonyl group (i.e. SO, or SO₂);
R^{b} independently represents hydrogen or C₁₋₄ alkyl; and
M represents hydrogen or a pharmaceutically acceptable cation; in cases where the molecule contains an internal base which would be protonated by a sulfonic acid, M may represent a negative charge (i.e., is optionally a negative charge).

An embodiment of the present invention (Embodiment E2) is a compound of Formula I, or a prodrug or pharmaceutically acceptable salt thereof, wherein:
R represents a seven, eight, or nine membered saturated or unsaturated ring optionally containing one to three heteroatoms independently selected from N, O and S, wherein the ring is optionally substituted with one or more R^{a} groups (e.g., R is optionally substituted with from 1 to 8 R^{a} groups, or is optionally substituted with from 1 to 6 R^{a} groups, or is optionally substituted with from 1 to 4 R^{a} groups, or is optionally substituted with from 1 to 3 R^{a} groups, or is optionally with 1 or 2 R^{a} groups, or is optionally mono-substituted with R^{a});
R¹ represents hydrogen or methyl;
R^{a} independently represents hydrogen, C₁₋₆ alkyl, halo; —(CH₂)ₙCN; —(CH₂)ₙNO₂; —(CH₂)ₙOR^{b}, —(CH₂)ₙSR^{b}; —(CH₂)ₙN(R^{b})₂; —(CH₂)ₙC(O)N(R^{b})₂; —(CH₂)ₙSO₂N(R^{b})₂; —(CH₂)ₙCO₂R^{b}; —(CH₂)ₙC(O)R^{b}; —(CH₂)ₙOCOR^{b}; —(CH₂)ₙNHCOR^{b}; —(CH₂)ₙNHC(O)₂R^{b}; —(CH₂)ₙNHSO₂R^{b}; —(CH₂)ₙC(NH)NH₂; —(CH₂)ₙC(NH)H; or two R^{a} groups on the same ring carbon atom are optionally taken together to form oxo; or two R^{a} groups on the same ring sulfur atom are optionally taken together with the sulfur to represent SO; or four R^{a} groups on the same ring sulfur atom are optionally taken together with the sulfur to represent SO₂;
n is 0-4 (i.e., each n is independently zero, 1, 2, 3, or 4);
each R^{b} independently represents hydrogen or C₁₋₄ alkyl; and
M represents hydrogen or a pharmaceutically acceptable cation; in cases where the molecule contains an internal base which would be protonated by a sulfonic acid, M may represent a negative charge.

The invention further relates to bacterial antibiotic resistance. More particularly, the invention relates to compositions and methods for overcoming bacterial antibiotic resistance. The patents and publications identified in this specification indicate the knowledge in this field and are hereby incorporated by reference in their entireties. In the case of inconsistencies, the present disclosure will prevail.

For purposes of the present invention, the following definitions will be used:

As used herein, the term “β-lactamase inhibitor” is used to identify a compound having a structure as defined herein, which is capable of inhibiting β-lactamase activity. Inhibiting β-lactamase activity means inhibiting the activity of a class A, C, or D β-lactamase. Preferably, for antimicrobial applications such inhibition should be at a 50% inhibitory concentration below 100 micrograms/mL, more preferably below 50 micrograms/mL and most preferably below 25 micrograms/mL. The terms “class A”, “class C”, and “class D” β-lactamases are understood by those skilled in the art and can be found described in Waley, The Chemistry of β-lactamase Page Ed., Chapman & Hall, London, (1992) 198-228.

As used herein, the term “β-lactamase” denotes a protein capable of inactivating a β-lactam antibiotic. In one preferred embodiment, the β-lactamase is an enzyme which catalyzes the hydrolysis of the β-lactam ring of a β-lactam antibiotic. In certain preferred embodiments, the β-lactamase is microbial. In certain other preferred embodiments, the β-lactamase is a serine β-lactamase. Examples of such preferred β-lactamases are well known and are disclosed in, e.g., Waley, The Chemistry of β-lactamase, Page Ed., Chapman & Hall, London, (1992) 198-228. In particularly preferred embodiments, the β-lactamase is a class C β-lactamase of Pseudomonas aeruginosa or of Enterobacter cloacae P99 (hereinafter P99 β-lactamase).

When any variable (e.g. R^{a} or R^{b}) occurs more than one time in any constituent, its definition on each occurrence is independent at every other occurrence. Also, combinations of substituents/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formula I.

As used herein, the term “organism” refers to any multicellular organism. Preferably, the organism is an animal, more preferably a mammal, and most preferably a human

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃CH₂—), in certain circumstances a bivalent linking moiety can be “alkyl,” in which case those skilled in the art will understand the alkyl to be a divalent radical (e.g., —CH₂CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

A number of moieties disclosed herein can exist in multiple tautomeric forms, all of which are intended to be encompassed by any given tautomeric structure. More particularly, all tautomeric forms of the compounds embraced by Formula I, whether individually or in mixtures, are within the scope of the present invention. It is further noted that compounds of the present invention having a hydroxy substituent on a carbon atom in a heteroaromatic ring or, more generally, on a ring or aliphatic carbon atom which is part of a double bond are understood to include compounds in which only the hydroxy is present, compounds in which only the tautomeric keto form (i.e., an oxo substitutent) is present, and compounds in which the keto and enol forms are both present.

The compounds of the invention contain chiral centers and, as a result of the selection of substituents and substituent patterns (e.g., on R), can have additional asymmetric centers, and thus can occur as mixtures of stereoisomers, or as individual diastereomers, or enantiomers. All isomeric forms of these compounds, whether individually or in mixtures, are within the scope of the present invention.

The term “alkyl” as employed herein refers to a monovalent straight or branched chain, saturated aliphatic hydrocarbon radical having from 1 to 12 carbon atoms, preferably 1-8 carbon atoms, more preferably 1-6 carbon atoms, and most preferably 1 to 4 carbon atoms. Examples of alkyl groups include, without limitation, methyl, ethyl, propyl, isopropyl, butyl, tert-butyl, isobutyl, pentyl, and hexyl.

The term “substituted alkyl” refers to an alkyl group as defined above substituted with one, two, three or four substituents (e.g., substituted with from 1 to 4 substituents, or from 1 to 3 substituents, or from 1 to 2 substituents, or 1 substituent—i.e., is mono-substituted).

The term “alkenyl” as employed herein refers to a monovalent straight or branched chain aliphatic hydrocarbon radical containing one carbon-carbon double bond and having from 2 to 12 carbon atoms, preferably 2 to 8 carbon atoms, more preferably 2 to 6 carbon atoms and most preferably 2 to 4 carbon atoms. Examples of alkenyl groups include, without limitation, vinyl(ethenyl), 2-propenyl, isopropenyl, and isobutenyl.

The term “substituted alkenyl” refers to an alkenyl group as defined above substituted with one, two, three, or four substituents (e.g., substituted with from 1 to 4 substituents, or from 1 to 3 substituents, or from 1 to 2 substituents, or 1 substituent—i.e., is mono-substituted).

The term “alkynyl” as employed herein refers to a monovalent straight or branched chain aliphatic hydrocarbon radical containing one carbon-carbon triple bond and having from 2 to 12 carbon atoms, preferably 2 to 8 carbon atoms, more preferably 2 to 6 carbon atoms and most preferably 2 to 4 carbon atoms. Examples of alkynyl groups include, without limitation, ethynyl and propynyl.

The term “substituted alkynyl” refers to an alkynyl group as defined above substituted with one, two, three or four substituents (e.g., substituted with from 1 to 4 substituents, or from 1 to 3 substituents, or from 1 to 2 substituents, or 1 substituent—i.e., is mono-substituted).

The term “cycloalkyl” as employed herein refers to a saturated cyclic hydrocarbon groups having 3 to 12, preferably 3 to 8 carbons, and more preferably 3 to 7 carbons. Examples of cycloalkyl groups include, without limitation, cyclopropyl, cyclobutyl, cyclopentyl, cyclohexyl, cycloheptyl, and cyclooctyl.

The term “substituted cycloalkyl” refers to a cycloalkyl as defined above substituted with one or more substituents (e.g., substituted with from 1 to 4 substituents, or from 1 to 3 substituents, or from 1 to 2 substituents, or 1 substituent—i.e., is mono-substituted).

The term “cycloalkenyl” refers to a mono-saturated cyclic hydrocarbon group having 4 to 12 carbons, preferably 5 to 8 carbons, and more preferably 5 to 7 carbons. Examples of cycloalkenyl groups include, without limitation, cyclopentenyl and cyclohexenyl

The term “substituted cycloalkenyl” refers to a cycloalkenyl as defined above substituted with one or more substituents (e.g., substituted with from 1 to 4 substituents, or from 1 to 3 substituents, or from 1 to 2 substituents, or 1 substituent—i.e., is mono-substituted).

An “aryl” group is a C₆-C₁₄ aromatic moiety comprising one to three aromatic rings. Preferably, the aryl group is a C₆-C₁₀ aryl group. Examples of aryl groups include, without limitation, phenyl, naphthyl, anthracenyl, and fluorenyl. A preferred aryl group is phenyl.

A “substituted aryl” group is an aryl group as defined above substituted with one or more substituents (e.g., substituted with from 1 to 4 substituents, or from 1 to 3 substituents, or with 1 or 2 substituents, or is mono-substituted). One class of substituted aryl is the “alkaryl” group which is an aryl group substituted with one or more alkyl groups (e.g., substituted with from 1 to 4 alkyl groups, or from 1 to 3 alkyl groups, or 1 or 2 alkyls, or 1 alkyl). Examples of alkaryl groups include, without limitation, tolyl, xylyl, mesityl, ethylphenyl, tert-butylphenyl, and methylnaphthyl

An “aralkyl” or “arylalkyl” group comprises an aryl group covalently linked to an alkyl group which is attached to the rest of the molecule. Preferably, the aralkyl group is —C₁₋₆ alkylene-C₆₋₁₀ aryl including, without limitation, benzyl, phenethyl, and naphthylmethyl.

A “substituted aralkyl” group is an aralkyl group as defined above substituted with one or more substituents (e.g., substituted with from 1 to 4 substituents, or from 1 to 3 substituents, or from 1 to 2 substituents, or 1 substituent—i.e., is mono-substituted), wherein the substitution is on either or both the alkyl and aryl moieties. In one embodiment, the substitution is present only on the aryl moiety.

The term “hydrocarbyl” refers to any group which is composed carbon and hydrogen, is saturated or unsaturated, is aliphatic or cyclic or contains both aliphatic and cyclic structures wherein the cyclic structure(s) can be a single ring or two or more rings which can be independent of, fused to, or bridged with each other. Examples of hydrocarbyl groups include, without limitation, alkyl, alkenyl, alkynyl, cycloalkyl, cycloalkenyl, alkyl substituted with cycloalkyl, alkyl substituted with cycloalkenyl, cycloalkyl substituted with alkyl, cycloalkenyl substituted with alkyl, aryl, arylalkyl, alkylaryl, and so forth.

The term “halohydrocarbyl” refers to a hydrocarbyl group as defined above substituted with one or more halogen atoms (e.g., substituted with from 1 to 4 substituents, or from 1 to 3 substituents, or from 1 to 2 substituents, or 1 substituent—i.e., is mono-substituted). More particularly, the term “haloalkyl” refers to an alkyl group as defined above substituted with one or more halogen atoms (e.g., substituted with from 1 to 4 substituents, or from 1 to 3 substituents, or from 1 to 2 substituents, or 1 substituent—i.e., is mono-substituted). Exemplary haloalkyl groups include, but are not limited to, CF₃, CH₂CF₃, CH₂F, and CHF₂.

The term heterocycle, heterocyclyl, or heterocyclic, as used herein, unless specifically stated otherwise, represents a stable 5- to 9-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocycle or heterocyclic includes heteroaryl moieties.

Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, between one and about three heteroatoms selected from the group consisting of N, 0, and S. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

In certain other preferred embodiments, the heterocyclic group is fused to an aryl or heteroaryl group. Examples of such fused heterocycles include, without limitation, tetrahydroquinolinyl and dihydrobenzofuranyl.

For purposes of this invention examples of seven, eight, or nine member saturated or unsaturated rings optionally containing one to three nitrogen, oxygen, or sulfur atoms include the aryl, heterocycloalkyl, heterocycle, heterocyclyl, heterocyclic, cycloalkyl and cycloalkenyl rings described herein. Examples of seven, eight, or nine member saturated or unsaturated rings include cycloheptenyl, cycloheptyl, cyclooctyl, cyclononyl, azepanyl, oxazepanyl, diazepanyl, thiazepanyl, azocanyl, oxazocanyl, diazocanyl and the like.

A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2,4-difluoro-3-propylphenyl. As another non-limiting example, substituted n-octyls include 2,4-dimethyl-5-ethyl-octyl and 3-cyclopentyloctyl. Included within this definition is the “oxo” substituent in which a methylene (—CH₂—) is substituted with oxygen to form carbonyl (—CO—).

Unless otherwise stated, as employed herein, when a moiety (e.g., alkyl, alkenyl, alkynyl, cycloalkyl, cycloalkenyl, aryl, heteroaryl, heterocyclyl, etc.) is described as “substituted” it is meant that the group has one or more non-hydrogen substituents (e.g., from one to four, or from one to three, or one or two, non-hydrogen substituents). Suitable substituents include, without limitation, halo, hydroxy, oxo (e.g., an annular —CH— substituted with oxo is —C(O)—), nitro, halohydrocarbyl (e.g., haloalkyl), hydrocarbyl (e.g., alkyl, alkenyl, cycloalkyl, aryl, or aralkyl), alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino, and
(b) C₁-C₆ alkyl or alkenyl or arylalkyl imino, carbamoyl, azido, carboxamido, mercapto, hydroxy, hydroxyalkyl, alkylaryl, arylalkyl, C₁-C₈ alkyl, SO₂CF₃, CF₃, SO₂Me, C₂-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkylcarbamoyl, C₂-C₁₅ N,N dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, or aryl, wherein each of the foregoing is further optionally substituted with one more moieties listed in (a), above.

The term “halogen” or “halo” as employed herein refers to chlorine, bromine, fluorine, or iodine. Preferred halogens are chlorine and fluorine.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

The term “heterocycloalkyl” refers to a cycloalkyl group (nonaromatic) in which one of the carbon atoms in the ring is replaced by a heteroatom selected from O, S or N, and in which up to three additional carbon atoms may be replaced by hetero atoms.

The term “heteroatom” means O, S or N, selected on an independent basis.

Alkoxy refers to an alkyloxy group (e.g., —O—C₁-C₄ alkyl).

Substitution by one or more named substituents on one or more atoms in one or more groups is permitted provided such substitution is chemically allowed and results in a stable compound.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis, 2^{nd} edition, Wiley, N.Y. (1991). Examples of suitable protecting groups are contained throughout the specification.

An embodiment of the present invention is a compound of Formula I, or a prodrug or a pharmaceutically acceptable salt thereof, wherein R is a 7-, 8-, or 9-membered saturated ring containing one nitrogen atom and a balance of carbon atoms; and all other variables are as defined in Embodiment E1 or Embodiment E2.

An embodiment of this invention is realized when R is a seven membered heterocyclic ring and all other variables are as described herein.

An embodiment of this invention is realized when R is a seven membered heterocyclic ring containing six carbons and one nitrogen and all other variables are as described herein.

Another embodiment of this invention is realized when R is an eight membered heterocyclic ring containing seven carbons and one nitrogen.

Still another embodiment of this invention is realized when R is a seven-membered heterocyclic ring containing five carbons and two nitrogens.

Still another embodiment of this invention is realized when R is an eight-membered heterocyclic ring containing six carbons and two nitrogens.

Still another embodiment of this invention is realized when R is a seven-membered heterocyclic ring containing five carbons, one nitrogen, and one oxygen.

Still another embodiment of this invention is realized when R is a nine-membered heterocyclic ring containing one nitrogen.

Another embodiment of the present invention is a compound of Formula I, or a prodrug or a pharmaceutically acceptable salt thereof, wherein R¹ is H; and all other variables are as defined in Embodiment E1 or Embodiment E2 or in any other embodiment described herein.

Yet another embodiment of this invention is realized when R¹ is methyl.

An embodiment of this invention is a compound of formula II or III:

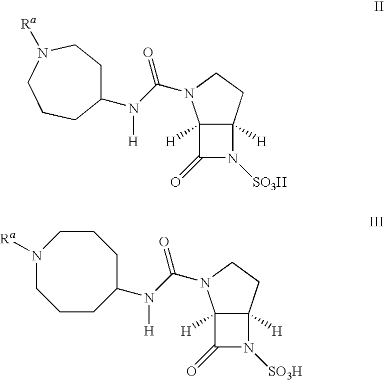

or a pharmaceutically acceptable salt thereof, wherein R^{a} is as defined in Embodiment E1, Embodiment E2, or as otherwise described herein. In a sub-embodiment of this embodiment, R^{a} is hydrogen, C₁₋₆ alkyl, —C(═NH)NH₂; or —C(═NH)H. In another sub-embodiment of this embodiment, R^{a} is selected from the group consisting of H and C₁₋₄ alkyl.

Another embodiment of this invention is a compound of Formula I, or a prodrug or pharmaceutically acceptable salt thereof, wherein:
R is an 7-, 8- or 9-membered saturated ring containing N(R^{a}) and optionally also containing either O or NH; wherein the two ring atoms adjacent and directly bonded to N(R^{a}) are carbon atoms and (i) one of the ring carbons directly bonded to the N(R^{a}) is optionally substituted with oxo or is optionally mono-substituted with methyl or is optionally di-substituted with methyl, or (ii) both of the ring carbons directly bonded to the N(R^{a}) are independently and optionally mono- or di-substituted with methyl; (and no other substitution is permitted in the ring R)
R¹ is hydrogen; and
R^{a} is hydrogen, C₁₋₄ alkyl, —(CH₂)₂₋₃OH, —(CH₂)₂₋₃O—C₁₋₃ alkyl, —(CH₂)₂₋₃NH₂, —(CH₂)₂₋₃N(H)—C₁₋₃ alkyl, —(CH₂)₂N(—C₁₋₃ alkyl)₂, —C(NH)NH₂, or —C(═NH)H;
and all other variables are as defined in Embodiment E1 or Embodiment E2 or as defined in any other embodiment described herein. In a sub-embodiment of this embodiment, R is an 7-, 8- or 9-membered saturated ring containing N(R^{a}), wherein R^{a} is hydrogen, CH₃, —(CH₂)₂OH, —(CH₂)₂NH₂, —(CH₂)₂N(H)CH₃, or —(CH₂)₂N(CH₃)₂.

A class of compounds of the present invention includes compounds of Formula I and pharmaceutically acceptable salts thereof, wherein R is:

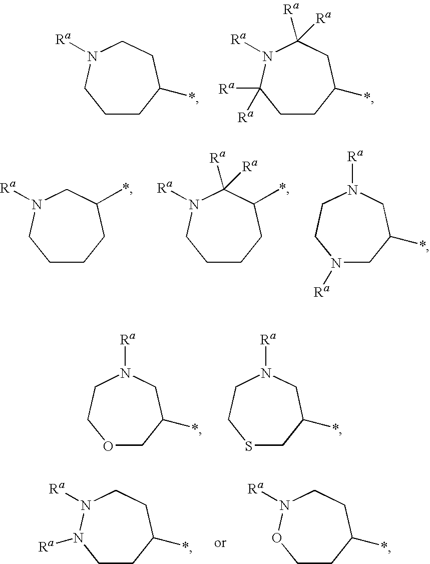

wherein the asterisk (*) at the end of the bond denotes the point of attachment of R to the rest of the compound;
R¹ is H;
each R^{a} which is a substituent on a ring N is independently selected from the group consisting of H, CH₃, —(CH₂)₂₋₃OH, —(CH₂)₂NH₂, —(CH₂)₂N(H)CH₃, —(CH₂)₂N(CH₃)₂, —(CH₂)₁₋₂C(O)NH₂, —(CH₂)₁₋₂C(O)N(H)CH₃, —(CH₂)₁₋₂C(O)N(CH₃)₂, and —CH(═NH);
each R^{a} which is a substituent on a ring carbon is independently H or CH₃ or, in the event that two R^{a} groups are on the same ring carbon atom, the two R^{a} groups are optionally taken together to form oxo; with the proviso that at least one R^{a} on a ring carbon is other than H; and
M is H.

The compounds of this invention can be combined with beta-lactam antibiotics such as imipenem, Primaxin® (combination of imipenem and cilastatin), ertapenem, meropenem, doripenem, biapenem, panipenem, Amoxicillin, Ticarcillin, Ampicillin, Cefoperazone, Piperacillin, and ceftazidime. Thus, another aspect of this invention is realized when the compounds of this invention are co-administered with a beta-lactam antibiotic.

Examples of compounds of this invention (optionally in the form of a pharmaceutically acceptable salt) are:

| Name | Structure |
|---|---|
| (1S,5R)-2-{[(4S)-azepan-4-ylamino]carbonyl}-7- oxo-2,6-diazabicyclo[3.2.0]heptane-6-sulfonic acid | 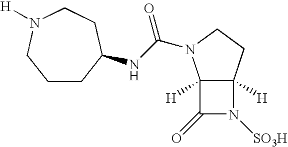 |
| (1S,5R)-2-{[(4R)-azepan-4-ylamino]carbonyl}-7- oxo-2,6-diazabicyclo[3.2.0]heptane-6-sulfonic acid | 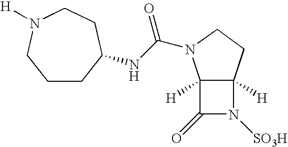 |
| (1S,5R)-2-{[(cycloheptylamino]carbonyl}-7-oxo- 2,6-diazabicyclo-[3.2.0]-heptane-6-sulfonic acid | 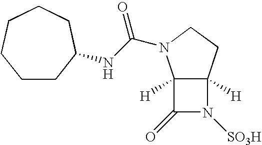 |
| (1S,5R)-2-{[(3S)-azepan-3-ylamino]carbonyl}-7- oxo-2,6-diazabicyclo[3.2.0]heptane-6-sulfonic acid | 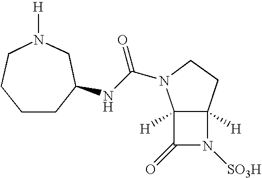 |
| (1S,5R)-7-oxo-2-({[(3S)-2-oxoazepan-3- yl]amino}carbonyl)-2,6- diazabicyclo[3.2.0]heptane-6-sulfonic acid | 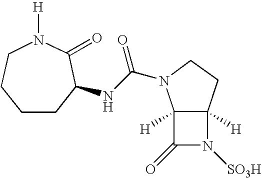 |
| (1S,5R)-2-[(1,4-diazepan-6-ylamino)carbonyl]-7- oxo-2,6-diazabicyclo[3.2.0]heptane-6-sulfonic acid | 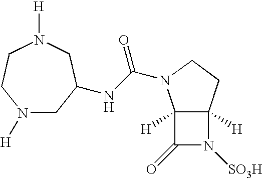 |
| (1S,5R)-2-{[(6R)-1,4-oxazepan-6- ylamino]carbonyl}-7-oxo-2,6- diazabicyclo[3.2.0]heptane-6-sulfonic acid | 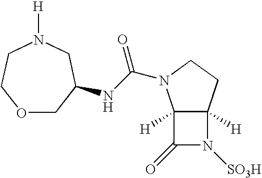 |
| (1S,5R)-2-{[(6S)-1,4-oxazepan-6- ylamino]carbonyl}-7-oxo-2,6- diazabicyclo[3.2.0]heptane-6-sulfonic acid | 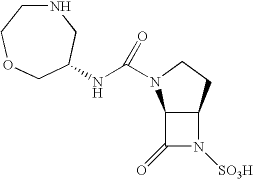 |
| (1S,5R)-2-({[(4S)-1-methylazepan-4- yl]amino}carbonyl)-7-oxo-2,6- diazabicyclo[3.2.0]heptane-6-sulfonic acid | 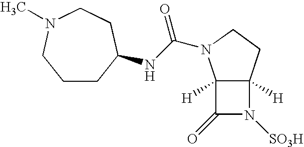 |
| (1S,5R)-2-({[(4S)-1-(2-hydroxyethyl)azepan-4- yl]amino}carbonyl)-7-oxo-2,6- diazabicyclo[3.2.0]heptane-6-sulfonic acid | 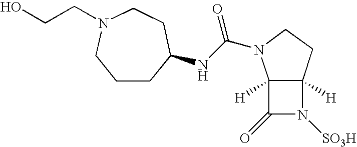 |
| (1S,5R)-2-({[(4S)-1-(3-hydroxypropyl)azepan-4- yl]amino}carbonyl)-7-oxo-2,6-diazabicyclo- [3.2.0]heptane-6-sulfonic acid | 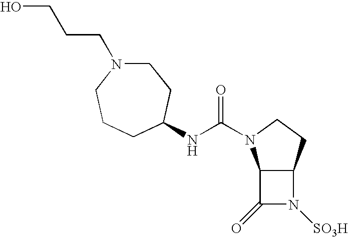 |
| (1S,5R)-2-[({(4S)-1-[2-(amino)ethyl]azepan-4- yl}amino)carbonyl]-7-oxo-2,6- diazabicyclo[3.2.0]heptane-6-sulfonic acid | 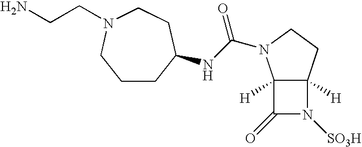 |
| (1S,5R)-2-[({(4S)-1-[2- (dimethylamino)ethyl]azepan-4- yl}amino)carbonyl]-7-oxo-2,6- diazabicyclo[3.2.0]heptane-6-sulfonic acid | 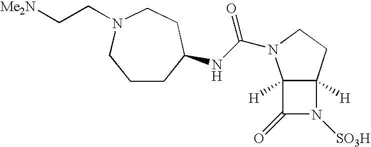 |
| (1S,5R)-7-oxo-2-{[(2,2,7,7-tetramethylazepan-4- yl)amino]carbonyl}-2,6-diazabicyclo-[3.2.0]- heptane-6-sulfonic acid | 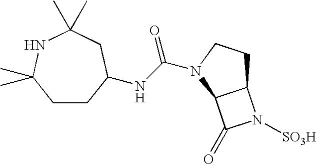 |
| (1S,5R)-2-[(azocan-5-ylamino)carbonyl]-7-oxo- 2,6-diazabicyclo-[3.2.0]-heptane-6-sulfonic acid | 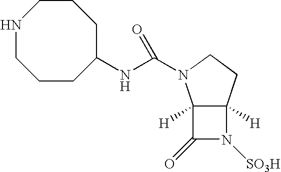 |
| (1S,5R)-2-[(azocan-4-ylamino)carbonyl]-7-oxo- 2,6-diazabicyclo[3.2.0]heptane-6-sulfonic acid (Isomer A) | 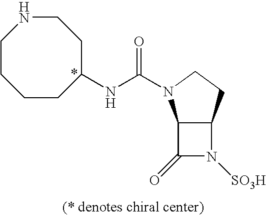 |
| (1S,5R)-2-[(azocan-4-ylamino)carbonyl]-7-oxo- 2,6-diazabicyclo[3.2.0]heptane-6-sulfonic acid (Isomer B) | 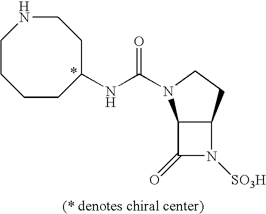 |
| (1S,5R)-2-{[azonan-5-ylamino]carbonyl}-7-oxo- 2,6-diazabicyclo[3.2.0]heptane-6-sulfonic acid (Isomer A) | 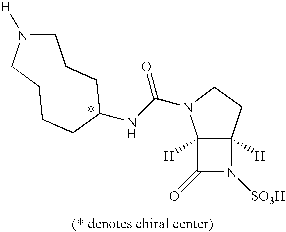 |
| (1S,5R)-2-{[azonan-5-ylamino]carbonyl}-7-oxo- 2,6-diazabicyclo[3.2.0]heptane-6-sulfonic acid (Isomer B) | 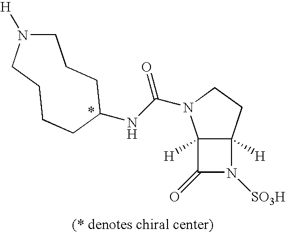 |
| (1S,5R)-7-oxo-2-{[(6R)-1,4-thiazepan-6- ylamino]carbonyl}-2,6- diazabicyclo[3.2.0]heptane-6-sulfonic acid | 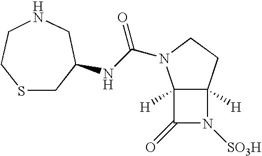 |
| (1S,5R)-2-({[(4S)-1-(2-amino-2-oxoethyl)azepan- 4-yl]amino}carbonyl)-7-oxo-2,6- diazabicyclo[3.2.0]heptane-6-sulfonic acid | 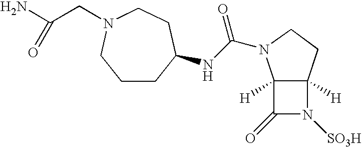 |
| (1S,5R)-2-({[(4S)-1-(iminomethyl)azepan-4- yl]amino}carbonyl)-7-oxo-2,6- diazabicyclo[3.2.0]heptane-6-sulfonic acid | 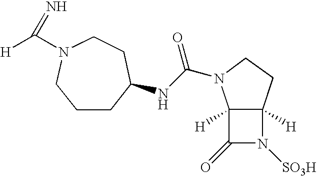 |
| (1S,5R)-7-oxo-2-({[(4S)-7-oxoazepan-4- yl]amino}carbonyl)-2,6- diazabicyclo[3.2.0]heptane-6-sulfonic acid | 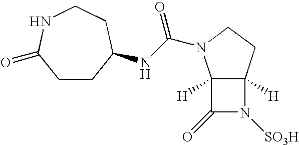 |
| (1S,5R)-7-oxo-2-({[(4R)-7-oxoazepan-4- yl]amino}carbonyl)-2,6- diazabicyclo[3.2.0]heptane-6-sulfonic acid | 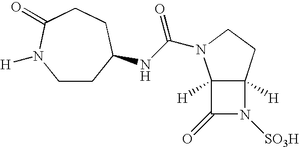 |
| (1S,5R)-2-[(1,2-diazepan-5-ylamino)carbonyl]-7- oxo-2,6-diazabicyclo[3.2.0]heptane-6-sulfonic acid | 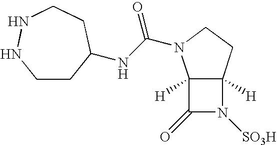 |
| (1S,5R)-2-{[(5R)-1,2-oxazepan-5- ylamino]carbonyl}-7-oxo-2,6- diazabicyclo[3.2.0]heptane-6-sulfonic acid | 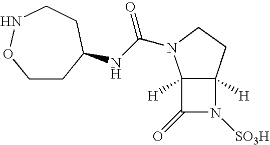 |
| (1S,5R)-2-({[4-(3- aminopropyl)cycloheptyl]amino}carbonyl)-7- oxo-2,6-diazabicyclo[3.2.0]heptane-6-sulfonic acid | 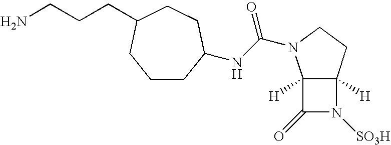 |
| (1S,5R)-2-({[(1S,4R)-4- aminocycloheptyl]amino}carbonyl)-7-oxo-2,6- diazabicyclo[3.2.0]heptane-6-sulfonic acid | 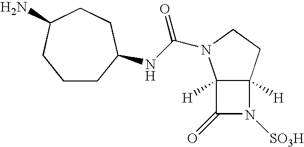 |
| (1S,5R)-2-[(1,5-diazocan-3-ylamino)carbonyl]-7- oxo-2,6-diazabicyclo[3.2.0]heptane-6-sulfonic acid | 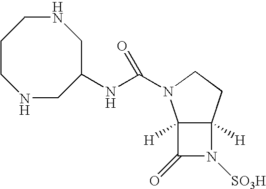 |
| (1S,5R)-2-{[(7R)-1,4-oxazocan-7- ylamino]carbonyl}-7-oxo-2,6- diazabicyclo[3.2.0]heptane-6-sulfonic acid | 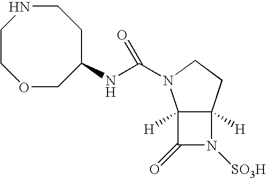 |
| (1S,5R)-7-oxo-2-{[(4R)-2,3,4,7-tetrahydro-1H- azepin-4-ylamino]carbonyl}-2,6- diazabicyclo[3.2.0]heptane-6-sulfonic acid | 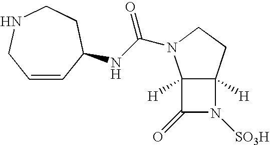 |

Another embodiment of the present invention is a compound selected from the group consisting of the compounds of Examples 1 to 31 (alternatively referred to more simply as Compounds 1 to 31) and pharmaceutically acceptable salts thereof.

Another embodiment of the present invention is a compound selected from the group consisting of compounds 1 to 20 and pharmaceutically acceptable salts thereof.

Still another embodiment of the present invention is (1S,5R)-2-{[(4S)-azepan-4-ylamino]carbonyl}-7-oxo-2,6-diazabicyclo[3.2.0]heptane-6-sulfonic acid (i.e., the compound of Example 1 or, more simply, “Compound 1”) or a pharmaceutically acceptable salt thereof.

Another embodiment of the present invention is a compound of Formula I, or a pharmaceutically acceptable salt thereof, as originally defined or as defined in any of the foregoing embodiments, sub-embodiments, aspects, or classes, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising an effective amount of a compound of Formula I as defined above, or a pharmaceutically acceptable salt thereof, and a pharmaceutically acceptable carrier.

(b) The pharmaceutical composition of (a), further comprising an effective amount of a β-lactam antibiotic.

(c) The pharmaceutical composition of (a), further comprising a β-lactam antibiotic and a DHP inhibitor.

(d) The pharmaceutical composition of (b), wherein the beta-lactam antibiotic is selected from the group consisting of imipenem, ertapenem, meropenem, doripenem, biapenem, panipenem, Amoxicillin, Ticarcillin, Ampicillin, Cefoperazone, Piperacillin, and ceftazidime.

(e) The pharmaceutical composition of (c), wherein the beta-lactam antibiotic is selected from the group consisting of imipenem, ertapenem, meropenem, doripenem, biapenem, panipenem, Amoxicillin, Ticarcillin, Ampicillin, Cefoperazone, Piperacillin, and ceftazidime and the DHP inhibitor is cilastatin or a pharmaceutically acceptable salt thereof.

(f) The pharmaceutical composition of (b), wherein the β-lactam antibiotic is imipenem.

(g) The pharmaceutical composition of (c), wherein the β-lactam antibiotic is imipenem and the DHP inhibitor is cilastatin or a pharmaceutically acceptable salt thereof.

(h) A combination of effective amounts of a compound of Formula I as defined above, or a pharmaceutically acceptable salt thereof, and a β-lactam antibiotic.

(i) A combination of effective amounts of a compound of Formula I as defined above, or a pharmaceutically acceptable salt thereof, a β-lactam antibiotic and a DHP inhibitor.

(j) The combination of (h), wherein the beta-lactam antibiotic is selected from the group consisting of imipenem, ertapenem, meropenem, doripenem, biapenem, panipenem, Amoxicillin, Ticarcillin, Ampicillin, Cefoperazone, Piperacillin, and ceftazidime.

(k) The combination of (i), wherein the beta-lactam antibiotic is selected from the group consisting of imipenem, ertapenem, meropenem, doripenem, biapenem, panipenem, Amoxicillin, Ticarcillin, Ampicillin, Cefoperazone, Piperacillin, and ceftazidime and the DHP inhibitor is cilastatin or a pharmaceutically acceptable salt thereof.

(l) The combination of (h), (i), (j) or (k), wherein the β-lactam antibiotic is imipenem.

(m) A method for treating a bacterial infection which comprises administering to a subject in need of such treatment a therapeutically effective amount of a compound of Formula I, or a prodrug or pharmaceutically acceptable salt thereof, optionally in combination with a beta-lactam antibiotic.

(n) A method for treating a bacterial infection which comprises administering to a subject in need of such treatment a therapeutically effective amount of a compound of Formula I, or a prodrug or pharmaceutically acceptable salt thereof, in combination with a beta-lactam antibiotic and a DHP inhibitor.

(o) A method for treating a bacterial infection which comprises administering to a subject in need of such treatment a therapeutically effective amount of the composition of (a), (b), (c), (d), (e), (f), or (g).

(p) A method for treating a bacterial infection which comprises administering to a subject in need of such treatment a therapeutically effective amount of the combination of (h), (i), (j), (k), or (l).

The present invention also includes a compound of Formula I, or a pharmaceutically acceptable salt thereof, (i) for use in, (ii) for use as a medicament for, or (iii) for use in the preparation (or manufacture) of a medicament for treating bacterial infection. In these uses, the compounds of the present invention can optionally be employed in combination with one or more β-lactam antibiotics and/or one or more DHP inhibitors.

Additional embodiments of the invention include the pharmaceutical compositions, combinations and methods set forth in (a)-(p) above and the uses set forth in the preceding paragraph, wherein the compound of the present invention employed therein is a compound of one of the embodiments or classes described above. The compound may optionally be used in the form of a prodrug or a pharmaceutically acceptable salt in these embodiments.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of Formula I or its salt and a pharmaceutically acceptable carrier and optionally one or more excipients, it is understood that the term “substantially pure” is in reference to a compound of Formula I or its salt per se; i.e., the purity of the active ingredient in the composition.

As indicated above, the compounds of the present invention can be employed in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). A suitable pharmaceutically acceptable salt is a salt formed by treating the compound of the invention (e.g., a compound of Formula I, II or III) with one molar equivalent of a mild base (e.g., sodium carbonate, sodium bicarbonate, potassium bicarbonate, or sodium acetate). In this case, M is a cation, such as Na⁺ in the event of treatment with a sodium base. Another suitable pharmaceutically acceptable salt is a zwitterion, which is an internal salt that can exist due to the presence of a basic nitrogen in R which is protonated by the sulfonic acid group present in the molecule. In this case, M is a negative charge. For compounds of the invention containing two basic nitrogens in R, still another pharmaceutically acceptable salt is a salt formed by treatment of the compound with a suitable amount of acid (e.g., hydrochloric acid, trifluoroacetic acid, methanesulfonic acid, or the like) such that one of the basic nitrogens in R is protonated by the sulfonic acid group present in the molecule (i.e., M=a negative charge) and the other basic nitrogen is protonated by the acid with the positive charge of the protonated N balanced by a suitable negative counterion (e.g., chloride, trifluoroacetate, methanesulfonate, or the like). Still another pharmaceutically acceptable salt for compounds of the invention containing two basic nitrogens in R can be obtained by treating the compound with sufficient acid (e.g., sulfuric acid, HCl, methanesolufonic acid, or TFA) such that the sulfonic acid group present in the molecule remains protonated (i.e., M=H) and the basic nitrogen is protonated and has associated therewith a suitable negative counterion (e.g., sulfonate). As is clear from the foregoing, the precise nature and type of pharmaceutically acceptable salt which can be obtained will depend upon the nature of the specific compound being treated (e.g., the presence or absence of basic nitrogens in R) and the treatment conditions employed; e.g., it will depend upon the choice and amount of the acid or base with which the compound is treated, the pH of the treating media, the amount and choice of buffer (if any), and the like. It is understood that the present invention encompasses all types and forms of pharmaceutically acceptable salts of the compounds of the present invention.

The present invention includes a crystalline form of Compound 1, pharmaceutical compositions containing the crystalline form, and methods of making and using the crystalline form. More particularly, the present invention includes a crystalline dihydrate of Compound 1. In one embodiment (alternatively referred to herein as “Embodiment C1”), the crystalline dihydrate is characterized by an X-ray powder diffraction pattern obtained using copper K_{α} radiation (i.e., the radiation source is a combination of Cu K_{α1} and K_{α2} radiation) which comprises 2Θ values (i.e., reflections at 2Θ values) in degrees of about 10.1, 10.8 and 15.3. In this embodiment and analogous embodiments which follow the term “about” is understood to modify each of the 2Θ values; i.e., the expression “about 10.1, 10.8 and 15.3” is short-hand for “about 10.1, about 10.8 and about 15.3”.

A second embodiment (Embodiment C2) is a crystalline dihydrate of Compound 1, which is characterized by an X-ray powder diffraction pattern obtained using copper K_{α} radiation which comprises 2Θ values in degrees of about 10.1, 10.8, 15.3, 15.8, 16.6 and 17.5.

A third embodiment (Embodiment C3) is a crystalline dihydrate of Compound 1, which is characterized by an X-ray powder diffraction pattern obtained using copper K_{α} radiation which comprises 2Θ values in degrees of about 10.1, 10.8, 15.3, 15.8, 16.6, 17.5, 18.6, 23.0 and 23.7.

A fourth embodiment (Embodiment C4) is a crystalline dihydrate of Compound 1, which is characterized by an X-ray powder diffraction pattern obtained using copper K_{α} radiation which comprises 2Θ values in degrees of about 10.1, 10.8, 13.3, 15.3, 15.8, 16.6, 17.0, 17.5, 18.6, 19.8, 20.2, 21.4, 21.7, 23.0, 23.7, 24.3, 25.1, 26.5, 26.7, 27.5, 27.8, 28.5, 29.1 and 29.9.

A fifth embodiment (Embodiment C5) is a crystalline dihydrate of Compound 1 as defined in any one of Embodiments C1 to C4, which is further characterized by a differential scanning calorimetry curve, obtained at a heating rate of 10° C./minute in a closed aluminum pan, exhibiting an endotherm with an onset temperature of about 101° C. and a peak temperature of about 110° C.

The crystalline dihydrate of Compound 1 as set forth in the foregoing embodiments C1 to C5 can alternatively be described in terms of the crystallographic d-spacings corresponding to the 2Θ reflections. The corresponding d-spacings are listed in Example 34 below.

A sixth embodiment (Embodiment C6) is crystalline dihydrate of Compound 1 as originally set forth or as defined in any of the foregoing embodiments C1 to C5, wherein the crystal form is substantially pure.

The crystalline dihydrate can be employed in compositions, combinations, methods of treatment, and uses as set forth above for compounds of Formula I generally.

The present invention also includes a process for preparing a crystalline dihydrate of Compound 1. More particularly, the present invention includes a process (referred to herein as “Process P1”) for preparing a crystalline dihydrate of Compound 1 as defined and described above, which comprises:

(A) adding a C₁₋₄ alkyl alcohol solvate of Compound 1 to a mixture comprising water and C₁₋₄ alkyl alcohol to provide a slurry;

(B) ageing the slurry of Step A, optionally with the addition of more C₁₋₄ alkyl alcohol to the slurry during the ageing; and

(C) isolating the crystalline dihydrate from the slurry.

The slurry formation in Step A is suitably conducted at a temperature in a range of from about 5° C. to about 30° C., is typically conducted at a temperature in a range of from about 20° C. to about 25° C., and is preferably conducted at a temperature of about 25° C. The addition of the alcohol solvate to the water-alcohol mixture in Step A is optionally but preferably conducted with agitation (e.g., stirring). The amount of water in the water-alcohol mixture in Step A to make the slurry is suitably in a range of from about 5 to about 25 volume percent (vol. %) based on the total of the separate volumes of water and alcohol employed in the mixture. For example if 5 L of water and 15 L of alcohol are employed to make the mixture in Step A, the amount of water is 25 vol. % The amount of water typically employed in the water-alcohol mixture in Step A is in a range of from about 10 vol. % to about 25 vol. % (e.g., 20 vol. %).

The amount of the alcohol solvate of Compound 1 employed in Step A is suitably in a range of from about 0.05 to about 0.2 grams per mL of water+alcohol, and is typically in a range of from about 0.05 to about 0.15 g/mL (e.g., about 0.1 g/mL).

The alcohol employed in the mixture in Step A and the alcohol in the solvate of Compound 1 are generally the same alcohol. The alcohol can be, for example, methanol, ethanol, or IPA. The alcohol is preferably IPA.

The slurry is suitably aged in Step B at a temperature in a range of from about 25° C. to about 60° C.; is typically aged at a temperature in a range of from about 35° C. to about 55° C., and is more typically aged at a temperature in a range of from about 35° C. to about 45° C.

The term “ageing” and variants thereof (e.g., “aged”) as used in Process P1 mean maintaining the slurry for a time and under conditions effective to provide a higher yield of the desired crystalline form compared to that which can be achieved in the absence of ageing.

Effective conditions include conducting the ageing in a suitable temperature range and optionally but preferably with a suitable degree of agitation (e.g., stirring). The ageing step is optional in the sense that at least some of the desired material forms during slurrying step A, but inclusion of an ageing step is preferred in order to improve, and preferably maximize, yield.

An additional portion or portions of alcohol (e.g., IPA) can optionally be added to the slurry during the ageing step, provided that the total amount of alcohol does not exceed about 95 vol. %. The alcohol can be added in a single charge or can be added in two or more increments during the ageing step. The alcohol acts as an anti-solvent in the crystallization process.

The isolation of crystalline dihydrate in Step C refers to the recovery of the resulting crystalline product from the slurry. Isolation of the crystalline product can be accomplished, for example, by cooling the aged slurry of Step B (e.g., from a temperature in the range of from about 35° C. to 45° C. to a temperature of from about 15° C. to about 25° C. separating the crystalline material by filtration, washing the filtered crystalline product with the slurrying agent (e.g., with an alcohol-water mixture), and then drying the washed product with low heat (e.g., at a temperature in a range of from about 30° C. to about 40° C.) and/or low vacuum.

Unless expressly stated to the contrary, all ranges set forth herein are inclusive. Thus, for example, when a temperature is said to be in a range of from about 5° C. to about 30° C., it means the temperature can be about 5° C. or about 30° C. or any temperature in between.

The term “about”, when modifying the quantity of a substance or composition, or the value of a physical property (e.g., the peak temperature in an endotherm in a DSC curve) of a substance or composition, or the value of a parameter characterizing a process (e.g., the temperature at which a process is conducted), or the like refers to variation in the numerical quantity that can occur, for example, through typical measuring, handling and sampling procedures involved in the preparation, characterization, and use of the substance or composition; through inadvertent error in these procedures; through differences in the manufacture, source, or purity of the ingredients employed to make or use the compositions or carry out the procedures; and the like. In one embodiment, the term “about” means the reported numerical value±10% thereof. In an aspect of this embodiment, the term “about” means the reported numerical value±5% thereof. In the particular case of the 2Θ values in degrees in an XRPD, the term “about” typically means the value±0.1.

Another aspect of the invention provides pharmaceutical compositions comprising a β-lactamase inhibitor of the invention and a pharmaceutically acceptable carrier or diluent. The characteristics of the carrier will depend on the route of administration. As used herein, the term “pharmaceutically acceptable” means a non-toxic material that does not interfere with the effectiveness of the biological activity of the active ingredient(s). The term “physiologically acceptable” refers to a non-toxic material that is compatible with a biological system such as a cell, cell culture, tissue, or organism. Thus, compositions and methods according to the invention may, in addition to the inhibitor, contain diluents, fillers, salts, buffers, stabilizers, solubilizers, and other materials well known in the art. The pharmaceutical composition of the invention may also contain other active factors and/or agents which enhance the inhibition of β-lactamases and/or DD-peptidases.

The term “administration” and variants thereof (e.g., “administering” a compound) in reference to a compound of Formula I mean providing the compound, or a prodrug or pharmaceutically acceptable salt thereof, to the individual in need of treatment. When a compound or a prodrug or salt thereof is provided in combination with one or more other active agents (e.g., a carbapenem antibiotic or a DHP inhibitor or both), “administration” and its variants are each understood to include provision of the compound or its prodrug or salt and other agents at the same time or at different times. When the agents of a combination are administered at the same time, they can be administered together in a single composition or they can be administered separately. It is understood that a “combination” of active agents can be a single composition containing all of the active agents or multiple compositions each containing one or more of the active agents. In the case of two active agents a combination can be either a single composition comprising both agents or two separate compositions each comprising one of the agents; in the case of three active agents a combination can be single composition comprising all three agents, three separate compositions each comprising one of the agents, or two compositions one of which comprises two of the agents and the other comprises the third agent; and so forth.

The terms “therapeutically effective amount” and “therapeutically effective period of time” are used to denote known treatments at dosages and for periods of time effective to show a meaningful patient benefit, i.e., healing of conditions associated with bacterial infection, and/or bacterial drug resistance. Preferable, such administration should be parenteral, oral, sublingual, transdermal, topical, intranasal, intratracheal, or intrarectal. When administered systemically, the therapeutic composition is suitably administered at a sufficient dosage to attain a blood level of inhibitor of at least about 1 microgram/m:, typically about 10 micrograms/mL, and more typically about 25 micrograms/mL. For localized administration, much lower concentrations than this may be effective, and much higher concentrations may be tolerated.

As employed herein, the term “pro-drug” refers to pharmacologically acceptable derivatives, e.g., esters and amides, such that the resulting biotransformation products of the derivative is the active drug. Pro-drugs are known in the art and are described generally in, e.g., Goodman and Gilman, “Biotransformation of Drugs”, in The Pharmacological Basis of Therapeutics, 8th Ed., McGraw Hill, Int. Ed. 1992, p. 13-15, which is hereby incorparated by reference in its entirely.

Compounds of the invention may be formulated by any method well known in the art and may be prepared for administration by any route, including, without limitation, parenteral, oral, sublingual, transdermal, topical, intranasan, intratracheal, or intrarectal. In certain particularly preferred embodiments, compounds of the invention are administered intravenously in a hospital setting. In certain other embodiments, administration may be preferably by the oral route.

The invention also provides methods for inhibiting bacterial growth, such methods comprising administering to a bacterial cell culture, or to a bacterially infected cell culture, tissue, or organism, a β-lactamase inhibitor of Formula (I), Formula (II) or Formula III as defined for the first aspect of the invention.

Preferably, the bacteria to be inhibited by administration of a β-lactamase inhibitor of the invention are bacteria that are resistant to β-lactam antibiotics. More preferably, the bacteria to be inhibited are β-lactamase positive strains that are highly resistant to β-lactam antibiotics. The terms “slightly resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:11.20-11.26 (1995)). Preferably, “highly resistant” bacterial strains are those against which the MIC of imipenem is >16 μg/mL. Preferably, “slightly resistant” bacterial strains are those against which the MIC of imipenem is >4 μg/mL.

The methods according to this aspect of the invention are useful for inhibiting bacterial growth in a variety of contexts. In certain preferred embodiments, the compound of the invention is administered to an experimental cell culture in vitro to prevent the growth of β-lactam resistant bacteria. In certain other preferred embodiments the compound of the invention is administered to an animal, including a human, to prevent the growth of β-lactam resistant bacteria in vivo. The method according to this embodiment of the invention comprises administering a therapeutically effective amount of a β-lactamase inhibitor and a β-lactam antibiotic according to the invention for a therapeutically effective period of time to an animal, including a human. Preferably, the β-lactamase inhibitor is administered in the form of a pharmaceutical composition-according to the second aspect of the invention.

The compounds may be used in combination with antibiotic agents for the treatment of infections caused by Class C-β-lactamase producing strains, in addition to those infections which are subsumed within the antibacterial spectrum of the antibiotic agent. Examples of class C-β-lactamase producing bacteria are Pseudomonas aeruginosa, Enterobacter cloacae, Klebsiella pneumoniae, Escherichia coli and Acinetobacter baumannii.

In accordance with the instant invention, it is generally advantageous to use a compound of formula I in admixture or conduction with a carbapenem, penicillin, cephalosporin or other β-lactam antibiotic or prodrug. It also advantageous to use a compound of formula I in combination with one or more β-lactam antibiotics because of the class C β-lactamase inhibitory properties of the compounds. In this case, the compound of formula I and the β-lactam antibiotic can be administered separately (at the same time or as different times) or in the form of a single composition containing both active ingredients.

Carbapenems, penicillins, cephalosporins and other β-lactam antibiotics suitable for co-administration with the compounds of Formula I, whether by separate administration or by inclusion in the compositions according to the invention, include both those known to show instability to or to be otherwise susceptible to class C-β-lactamases and also known to have a degree of resistance to class C β-lactamase.

When the compounds of Formula I are combined with a carbapenem antibiotic, a dehydropeptidase (DHP) inhibitor may also be combined. Many carbapenems are susceptible to attack by a renal enzyme known as DHP. This attack or degradation may reduce the efficacy of the carbapenem antibacterial agent. Inhibitors of DHP and their use with carbapenems are disclosed in, e.g., (European Patent 0007614, filed Jul. 24, 1979 and application number 82107174.3, filed Aug. 9, 1982 and incorporated by reference herein in its entirety. A preferred DHP inhibitor is 7-(L-2-amino-2-carboxyethylthio)-2-(2,2-dimethylcyclopropanecarboxamide)-2-heptenoic acid or a useful salt thereof. Thus, compounds of the present invention in combination with a carbapenem such as imipenem and a DHP inhibitor such as, cilastatin is contemplated within the scope of this invention.

Examples of carbapenems that may be co-administered with the compounds of formula I include imipenem, meropenem, biapenem, (4R, 5S, 6S)-3-[3S, 5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S, 5R, 6S)-2-(4-(2-(((carbamoylmethy diazoniabicyclo[2.2.2]oct-1-yl)-ethyl(1,8-naphthosultam)methyl)-6-[1 (R)-hydroxyethyl]-1-methylcarbapen-2-em-3-carboxylate chloride, BMS181139 ([4R-[4alpha,5beta,6beta(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), BO2727 ([4R-3[3S*,5S*(R*)], 4alpha,5beta,6beta(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0] hept-2-ene-2-carboxylic acid monohydrochloride), E1010 ((1R, 5S, 6S)-6-[1(R)-hydroxymethyl]-2-[2(S)-[1(R)-hydrocy-1-[pyrrolidin-3(R)-yl]methyl]pyrrolidin-4(S)-ylsulfanyl]-1-methyl-1-carba-2-penem-3-carboxylic acid hydrochloride) and S4661 ((1R,5S,6S)-2-[(3S,5S)-5-(sulfamoylaminomethyl)pyrrolidin-3-yl]thio-6-[(1R)-1-hydroxyethyl]-1-methylcarbapen-2-em-3-carboxylic acid), (1S,5R,6S)-1-methyl-2-{7-[4-(aminocarbonylmethyl)-1,4-diazoniabicyclo(2.2.2)octan-1yl]-methyl-fluoren-9-on-3-yl}-6-(1R-hydroxyethyl)-carbapen-2-em-3 carboxylate chloride.

Examples of penicillins suitable for co-administration with the compounds according to the invention include benzylpenicillin, phenoxymethylpenicillin, carbenicillin, azidocillin, propicillin, ampicillin, amoxicillin, epicillin, ticarcillin, cyclacillin, pirbenicillin, azloccillin, mezlocillin, sulbenicillin, piperacillin, and other known penicillins. The penicillins may be used in the form of pro-drugs thereof, for example as in vivo hydrolysable esters, for example the acetoxymethyl, pivaloyloxymethyl, α-ethoxycarbonyloxy-ethyl and phthalidyl esters of ampicillin, benzylpenicillin and amoxicillin; as aldehyde or ketone adducts of penicillins containing a 6-α-aminoacetamido side chain (for example hetacillin, metampicillin and analogous derivatives of amoxicillin); and as esters of carbenicillin and ticarcillin, for example the phenyl and indanyl α-esters.

Examples of cephalosporins that may be co-administered with the compounds according to the invention include, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefinetazole, cefotaxime, ceftriaxone, and other known cephalosporins, all of which may be used in the form of pro-drugs thereof.

Examples of β-lactam antibiotics other than penicillins and cephalosporins that may be co-administered with the compounds according to the invention include aztreonam, latamoxef (Moxalactam-trade mark), and other known β-lactam antibiotics such as carbapenems like imipenem, meropenem or (4R, 5S, 6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid, all of which may be used in the form of pro-drugs thereof.

Preferred carbapenems are imipenem, meropenem and (4R, 5S, 6S)-3-[(3S,5S)-5-(3-carboxyphenylcarbamoyl)pyrrolidin-3-ylthio]-6-(1R)-1-hydroxyethyl]-4-methyl-7-oxo-1-azabicyclo[3.2.0]hept-2-ene-2-carboxylic acid.

Particularly suitable penicillins for co-administration with the compounds according to the invention include ampicillin, amoxicillin, carbenicillin, piperacillin, azlocillin, mezlocillin, and ticarcillin. Such penicillins may be used in the form of their pharmaceutically acceptable salts, for example their sodium salts. Alternatively, ampicillin or amoxicillin may be used in the form of fine particles of the zwitterionic form (generally as ampicillin trihydrate or amoxicillin trihydrate) for use in an injectable or infusable suspension, for example, in the manner described herein in relation to the compounds of formula I. Amoxicillin, for example in the form of its sodium salt or the trihydrate, is particularly preferred for use in compositions according to the invention.

Particularly suitable cephalosporins for co-administration with the compounds according to the invention include cefotaxime, ceftriaxone and ceftazidime, which may be used in the form of their pharmaceutically acceptable salts, for example their sodium salts.

In certain preferred embodiments of the method according to this aspect of the invention, a β-lactamase inhibitor according to the invention is co-administered with an antibiotic. Preferably, such co-administration produces a synergistic effect. As employed herein, the terms “synergy” and “synergistic effect” indicate that the effect produced when two or more drugs are co-administered is greater than would be predicted based on the effect produced when the compounds are administered individually. While not wishing to be bound by theory, the present inventors believe that the β-lactamase inhibitors according to the invention act to prevent degradation of β-lactam antibiotics, thereby enhancing their efficacy and producing a synergistic effect. In particularly preferred embodiments of the invention, therefore, the co-administered antibiotic is a β-lactam antibiotic. For purposes of this invention, the term “co-administered” is used to denote simultaneous or sequential administration.

The term “antibiotic” is used herein to describe a compound or composition which decreases the viability of a microorganism, or which inhibits the growth or proliferation of a microorganism. “Inhibits the growth or proliferation” means increasing the generation time by at least 2-fold, preferably at least 10-fold, more preferably at least 100-fold, and most preferably indefinitely, as in total cell death. As used in this disclosure, an antibiotic is further intended to include an antimicrobial, bacteriostatic, or bactericidal agent. Non-limiting examples of antibiotics useful according to this aspect of the invention include penicillins, cephalosporins, carbapenems, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, and others. The term “β-lactam antibiotic” is used to designate compounds with antibiotic properties containing a β-lactam functionality. Non-limiting examples of β-lactam antibiotics useful according to this aspect of the invention include penicillins, cephalosporins, penems, carbapenems, and monobactams.

Abbreviations employed herein include the following: ACN=acetonitrile; BLI=beta-lactamase inhibitor; Bn=benzyl; BOC (or Boc)=t-butyloxycarbonyl; BSA=bovine serum albumin; CBZ or Cbz=benzyloxycarbonyl; DCC=dicyclohexyl carbodiimide; DCM=dichloromethane; DHP=dehydropeptidase; DIAD=diisopropylazodicarboxylate; DIBAL-H=diisobutylaluminum hydride; DMF=dimethylformamide; DMSO=dimethyl sulfoxide; DSC=differential scanning calorimetry; EDC=1-ethyl-3-(3-dimethylaminopropyl) carbodiimide; eq(s).=equivalent(s); Et=ethyl; EtOAc=ethyl acetate; HPLC=high performance liquid chromatography; IPA=isopropyl alcohol; IPAc=isopropyl acetate; KF=Karl Fisher titration for water; KRED-112 =ketoreductase 112; LC-MS=liquid chromatography-mass spectroscopy; Me=methyl; MIC=minimum inhibitory concentration; MP-TMT=macroporous polystyrene-2,4,6-trimercaptotriazine; Ms=mesyl or methanesulfonyl; NADP=nicotinamide ahenine dinucleostide phosphate; NADPH=reduced form of NADP; NMR=nuclear magnetic resonance; PDH-101=phosphite dehydrogenase 101; Ph=phenyl; PMB=para-methoxybenzyl; SFC=supercritical fluid chromatography; TFA=trifluoroacetic acid; TGA=thermogravimetric analysis; TLC=thin layer chromatography; TsOH=p-toluenesulfonic acid; XRPD=X-ray powder diffraction.

Generally, the compounds of the invention can be routinely synthesized using techniques known to those skilled in the art (see U.S. Pat. No. 5,698,577 and U.S. Pat. No. 5,510,343, both incorporated herein by reference in their entireties) in conjunction with the teachings herein.

The following examples are intended to further illustrate certain preferred embodiments of the invention, and are not intended to limit the scope of the invention.

The compounds of the present invention are prepared by reacting bridged monobactam intermediate A with a suitably protected, activated side chain precursor B as illustrated in Scheme 1.

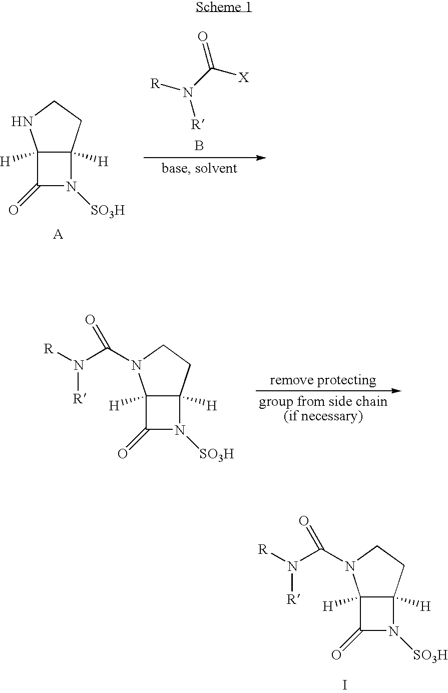

The bridged monobactam intermediate A can be obtained in accordance with Heinze-Krauss et al J. Med. Chem. 1998, 41, 3961, the teachings of which are incorporated herein by reference. Alternatively, intermediate A may be prepared by reduction of the related N-hydroxy analog disclosed by Miller et al Tet. Lett. 1997, 38: 167) using samarium iodide, Raney nickel, or the like followed by sulfonylation of the lactam nitrogen using techniques that are well known to those skilled in the art.

Activated side chain precursor B is obtained by reaction of the side chain amine RR¹NH with an activating reagent such as phosgene, p-nitrophenylchloroformate, di-(N-succinimidyl)-carbonate or the like in a solvent such as acetonitrile, ether, dichloromethane, or the like for one to twenty-four hours at a temperature between 0° C. and room temperature. In some cases, it may be advantageous or even necessary to add one molar equivalent of a base such as triethylamine, pyridine, or the like to the reaction mixture. Intermediate B can be isolated from the reaction mixture using standard techniques known to those skilled in the art and may be used without purification or, if desired, purified by standard methods such as crystallization or chromatography. Reagent B thus obtained may be reacted with bridged monobactam intermediate A in a solvent such as water, methanol, acetonitrile, or the like at a temperature ranging from 0° C. to 35° C. One molar equivalent of a base such as sodium bicarbonate, triethylamine, pyridine, or the like is generally present during the reaction but may be omitted in some cases. The resulted acylated monobactam may be purified by HPLC using techniques known to those skilled in the art. In cases where there is no protecting group in the side chain, the product of the acylation reaction is a compound of the present invention having formula I. When a protecting group such as a benzyl amine or ether, a t-butoxycarbonyl amine, a benzyloxycarbonyl amine, or the like is present in the side chain, it is necessary to remove the protecting group to afford the compound of formula I. Techniques for removal of protecting groups are well known to those skilled in the art.

In some cases, it may be desirable to protect the sulfonic acid moiety of the bridged mono-bactam as in intermediate C (Scheme 2). In this instance, the acylation of the bridged monobactam intermediate C with side-chain precursor B proceeds exactly as described above for the acylation of intermediate A but it is necessary to remove the sulfonic acid protecting group in a subsequent step. In some cases, the sulfonic acid protecting group may be removed concurrently with a side chain protecting group. Purification of the final product by HPLC using techniques known to those skilled in the art affords the final product of formula I.

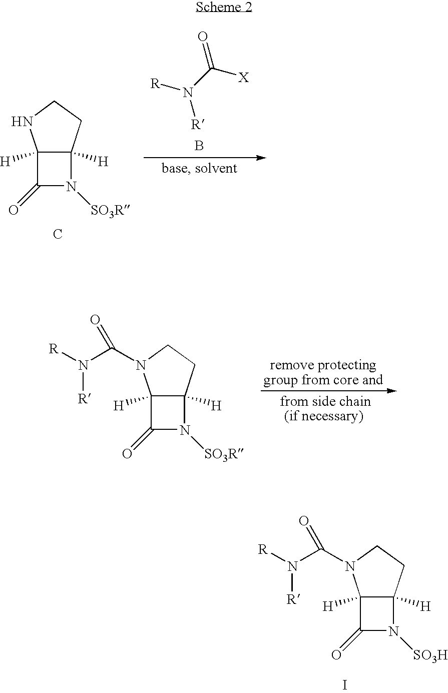

In some instances, it is desirable to activate the bridged monobactam and add the side chain to the activated monobactam intermediate D (Scheme 3). The activated bridged monobactam intermediate D is prepared by reaction of the protected monobactam B with an activating reagent such as phosgene, p-nitrophenylchloroformate, di-(N-succinimidyl)-carbonate in a solvent such as acetonitrile, ether, dichloromethane, or the like for one to twenty-four hours at a temperature between 0° C. and room temperature. In some cases, it may be advantageous or even necessary to add one molar equivalent of a base such as triethylamine, pyridine, or the like to the reaction mixture. Intermediate D can be isolated from the reaction mixture using standard techniques known to those skilled in the art and may be used without purification or, if desired, purified by standard methods such as crystallization or chromatography. Reagent D thus obtained may be reacted with the side chain amine E in a solvent such as water, methanol, acetonitrile, or the like at a temperature ranging from 0° C. to 35° C. One molar equivalent of a base such as sodium bicarbonate, triethylamine, pyridine, or the like is generally present during the reaction but may be omitted in some cases. The resulted acylated monobactam may be purified by HPLC using techniques known to those skilled in the art. Removal of protecting groups from the monobactam core (and the side chain, if any protecting groups are present in the side chain) affords the compound of formula I. Techniques for removal of protecting groups are well known to those skilled in the art.

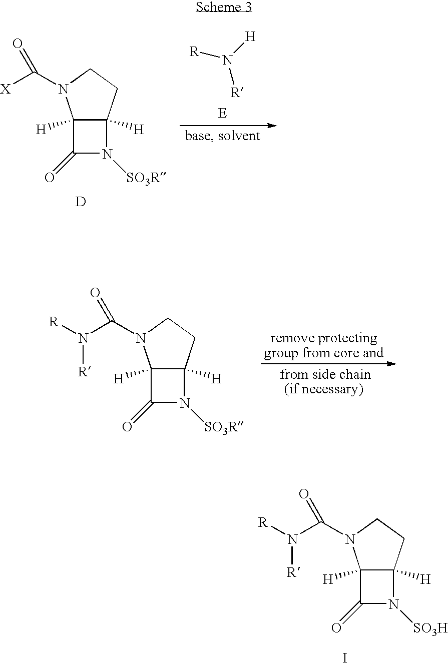

Alternatively, the compounds of the present invention may be synthesized from commercially available L-3-hydroxy-proline as outlined in Scheme 4. Reaction of 3-hydroxy proline with a suitably activated side chain precursor such as B in a solvent such as water, methanol, acetonitrile, or the like at a temperature ranging from 0° C. to 35° C. affords the acylated pyrrolidine. One molar equivalent of a base such as sodium bicarbonate, triethylamine, pyridine, or the like is generally present during the reaction but may be omitted in some cases. The resulted acylated pyrrolidine may be purified using chromatographic techniques known to those skilled in the art. Reaction of the carboxyl group of the acylated pyrrolidine intermediate with the amine of a protected sulfamate R″OSO₂NH₂ where R″ is a sulfonate protecting group such as triphenylmethyl, benzyl, t-butyl or the like in the presence of a coupling agent such as DCC, EDC or the like in a solvent such as dichloromethane, ether, tetrahydrofuran, or the like at a temperature ranging from 0° C. to 35° C. then affords the cyclization precursor F. Intermediate F is hydrolytically unstable and is generally cyclized immediately using a Mitsunobu cyclization procedure. Thus, a relatively dilute solution of intermediate F in a solvent such as dichloromethane, ether, tetrahydrofuran, or the like is treated with an azodicarboxylate such as DIAD or the like and a phosphine such as triphenylphosphine or the like at a temperature ranging from 0° C. to 35° C. Removal of the sulfamate protecting group and any protecting group such as a benzyl amine or ether, a t-butoxycarbonyl amine, a benzyloxycarbonyl amine, or the like present in the side chain, using techniques that are well known to those skilled in the art affords the compound of formula I.

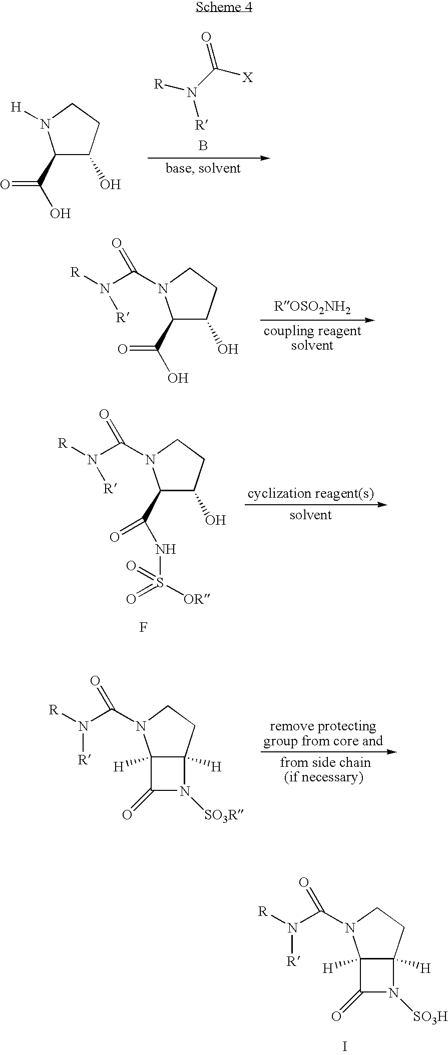

Alternatively, the compounds of the present invention may be synthesized from commercially available L-3-hydroxy-proline as outlined in Scheme 5. Reaction of 3-hydroxy proline with a suitably activated side chain precursor such as B in a solvent such as water, methanol, acetonitrile, or the like at a temperature ranging from 0° C. to 35° C. affords the acylated pyrrolidine. One molar equivalent of a base such as sodium bicarbonate, triethylamine, pyridine, or the like is generally present during the reaction but may be omitted in some cases. The resulted acylated pyrrolidine may be purified using chromatographic techniques known to those skilled in the art. Reaction of the carboxyl group of the acylated pyrrolidine intermediate with the amine of a protected sulfhydrylamine R″SNH₂ where R″ is a sulfur protecting group such as triphenylmethyl, benzyl, t-butyl or the like in the presence of a coupling agent such as DCC, EDC or the like in a solvent such as dichloromethane, ether, tetrahydrofuran, or the like at a temperature ranging from 0° C. to 35° C. then affords the cyclization precursor G. Intermediate G is generally cyclized immediately using a Mitsunobu cyclization procedure. Thus, a relatively dilute solution of intermediate G in a solvent such as dichloromethane, ether, tetrahydrofuran, or the like is treated with an azodicarboxylate such as DIAD or the like and a phosphine such as triphenylphosphine or the like at a temperature ranging from 0° C. to 35° C. Removal of the sulfur protecting group and oxidation to the sulfonic acid using a suitable oxidizing reagent such as bleach, oxygen, or the like followed by removal of any protecting group present in the side chain, using techniques that are well known to those skilled in the art affords the compound of formula I.

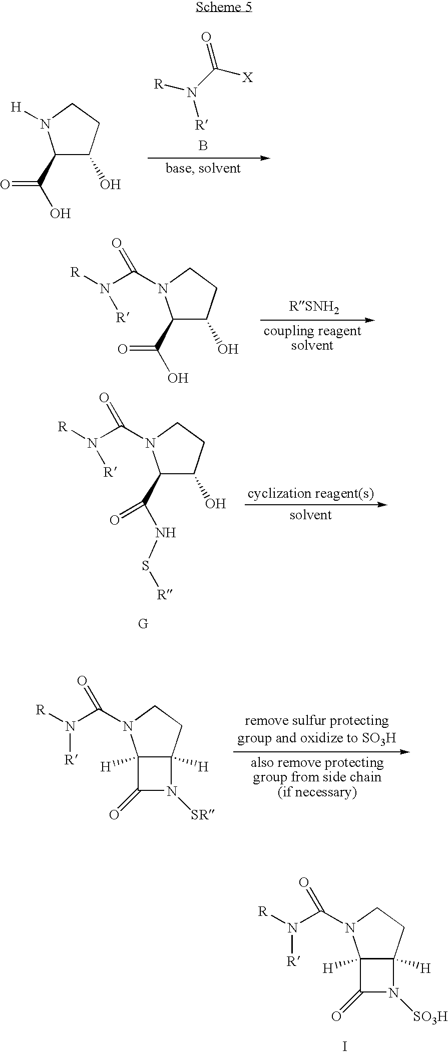

In certain other preferred embodiments, compounds of Formula I, II, or III are synthesized by more specific or less general chemistry which are exemplified in the experimental section.

### Preparative Example 1

### (1S,5R)-7-oxo-2,6-diazabicyclo[3.2.0]heptane-6-sulfonic acid

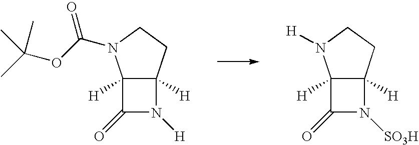

A solution of tert-butyl (1S,5R)-7-oxo-2,6-diazabicyclo[3.2.0]heptane-2-carboxylate (0.77 g, 3.6 mmol; J. Med. Chem. 1998, 41: 3961) in dimethylformamide (8 mL) was cooled to 0° C. and a solution of sulfur trioxide dimethylformamide complex (0.64 g, 4.2 mmol) was added dropwise. The resulting mixture was stirred at 0° C. for four hours then allowed to stand at 0° C. overnight. The reaction mixture was concentrated under vacuum and the oily residue was redissolved in dichloromethane (10 mL) and cooled to 0° C. Trifluoroacetic acid (5 mL) was added and the reaction mixture warmed to room temperature. After 2.5 hours at room temperature, the reaction was concentrated in vacuum and the residue was triturated with ether to afford an insoluble tan solid which was redissolved in water and lyophilized overnight. The resulting tan solid was purified by chromatography on MCI GEL® CHP20P (Supelco; a polyaromatic absorbent resin) eluted with water to afford the title compound as a solid (0.65 g, 94%).

### Preparative Example 2

### Benzyl (4S)-4-[(tert-butyl-(R)-sulfinyl)amino]azepane-1-carboxylate and benzyl (4R)-4-[(tert-butyl-(R)-sulfinyl)amino]azepane-1-carboxylate

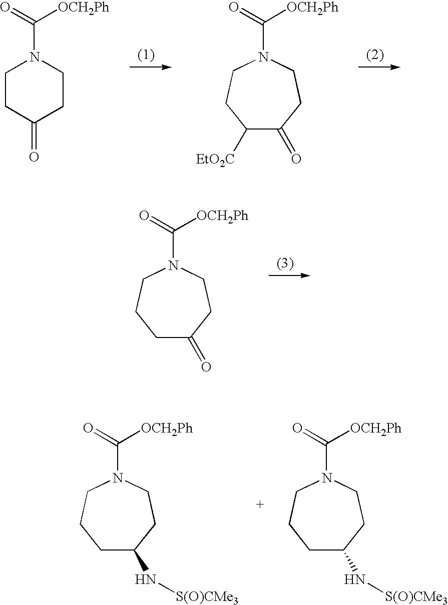

### Step 1: 1-Benzyl 4-ethyl 5-oxoazepane-1,4-dicarboxylate

To a solution of N-benzyloxycarbonyl-4-piperidone (1.27 mL, 6.5 mmol) in ether (15 mL) at −40° C. was added boron trifluoride etherate (0.98 mL, 7.75 mmol) dropwise under nitrogen followed by dropwise addition of a solution of ethyl diazoacetate (0.80 mL, 7.75 mmol) in ether (5 mL) over 15 minutes. After 1 hour, the reaction was poured into ice/saturated sodium bicarbonate. The organic phase was collected, washed with brine, dried over sodium sulfate, and concentrated under vacuum to afford the title compound as a yellow oil (2.05 g, 99%). The crude product was used without purification in the next step.

### Step 2: Benzyl 4-oxoazepane-1-carboxylate

To a solution of 1-benzyl 4-ethyl 5-oxoazepane-1,4-dicarboxylate (2.05 g, 6.4 mmol) in tetrahydrofuran (2 mL) was added a solution of potassium carbonate (1.95g, 14.15 mmol) in water (24 mL). The reaction was heated to reflux and refluxed for 3 hours then cooled to 0° C. and diluted with ethyl acetate. The mixture was acidified with stirring to pH 1 by addition of 2N HCl and the layers were separated. The organic layer was washed with brine, dried over sodium sulfate, and concentrated under vacuum to afford the title compound as a pale yellow oil (1.15 g, 72%).

### Step 3: Benzyl (4S)-4-[(tert-butyl-(R)-sulfinyl)amino]azepane-1-carboxylate and benzyl (4S)-4-[(tert-butyl-(R)-sulfinyl)amino]azepane-1-carboxylate:

To a solution of titanium (IV) ethoxide (1.46 mL, 7 mmol) and benzyl 4-oxoazepane-1-carboxylate (1.05 g, 4.2 mmol) in anhydrous tetrahydrofuran (12 mL) was added (R)-(+)-tert-butanesulfinamide (0.43 g, 3.55 mmol; Acssys Pharmatech) under nitrogen and the reaction was heated to 65° C. for 3 hours. The resulting solution was cooled to room temperature then to 0° C. and cannulated into a mixture of sodium borohydride (0.53 g, 14 mmol) in anhydrous tetrahydrofuran (5 mL) at −5° C. After 1.5 hours at −5° C. the reaction was quenched with methanol at 0° C. The resulting mixture was poured into brine and stirred vigorously. The resulting thick white slurry was filtered through Celite. The Celite pad was washed well with ethyl acetate and the filtrate was collected, washed with brine, dried over sodium sulfate, filtered, and concentrated under vacuum. The residue was purified by chromatography on silica gel eluted initially 15% ethyl acetate in hexane followed by 100% ethyl acetate followed by 10% methanol in dichloromethane to give the title compounds as a pale yellow oil (0.943 g). The diastereomers were separated by chromatography on a Chiralcel OJ semi-prep column eluted with 15% ethyl acetate in heptane to afford benzyl (4S)-4-[(tert-butyl (R)-sulfinyl)amino]azepane-1-carboxylate (0.455 g, 36%) and benzyl (4R)-4-[(tert-butyl-(R)-sulfinyl)amino]azepane-1-carboxylate (0.396 g, 32%).

### Preparative Example 3

### Benzyl (4S)-4-({[(2,5-dioxopyrrolidin-1-yl)oxy]carbonyl}amino)azepane-1-carboxylate

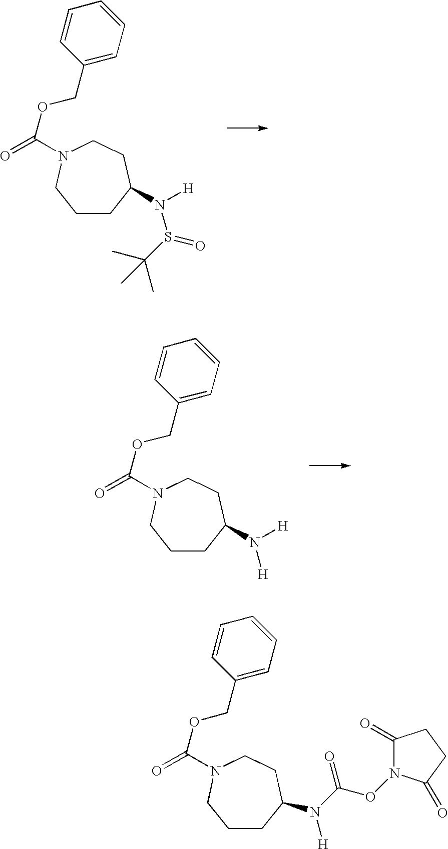

### Step 1: Benzyl (4S)-4-aminoazepane-1-carboxylate

To a solution of benzyl (4S)-4-[(tert-butyl (R)-sulfinyl)amino]azepane-1-carboxylate (0.40 g, 1.12 mmol) in methanol (5 mL) was added 4N hydrochloric acid in dioxane (0.28 mL, 1.12 mmol). The mixture was stirred for 90 minutes at room temperature then concentrated under vacuum. The residue was triturated with ether and dried under vacuum to afford the title compound as a pale yellow oil (0.32 g) which was used in the next step without further purification.

### Step 2: Benzyl (4S)-4-({[(2,5-dioxopyrrolidin-1-yl)oxy]carbonyl}amino)-azepane-1-carboxylate

To a solution of benzyl (4S)-4-aminoazepane-1-carboxylate (0.32 g, 1.12 mmol) in acetonitrile (5 mL) was added triethylamine (0.16 mL, 1.12 mmol) followed by N—N′-disuccinimidyl carbonate (0.29 g, 1.14 mmol) at room temperature under nitrogen. After 4 hours, the reaction mixture was concentrated under vacuum and purified by reverse-phase HPLC to afford the title compound as a white solid (0.322 g, 74%) after lyophilization.

### Preparative Example 4

### Benzyl (4R)-4-({[(2,5-dioxopyrrolidin-1-yl)oxy]carbonyl}amino)azepane-1-carboxylate

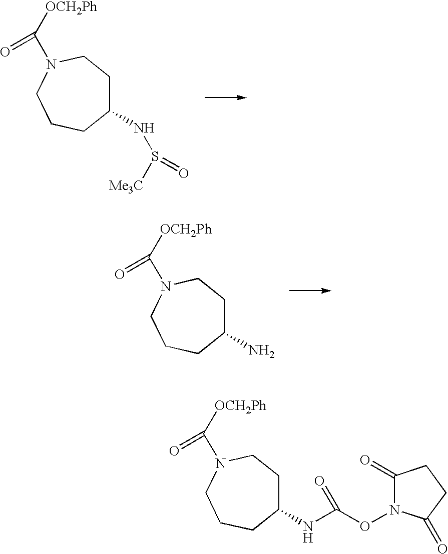

Using the procedure outlined in Preparative Example 3 for the enantiomeric product, the title compound was obtained as a white solid after lyophilization.

### Preparative Example 5

### (3S)-1-Benzylazepan-3-amine

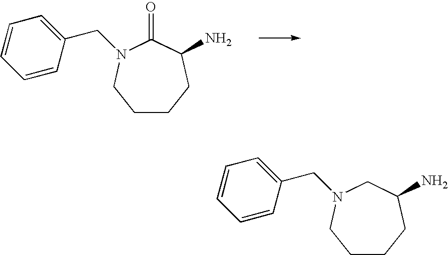

To a solution of (3S)-3-amino-1-benzylazepan-2-one (Astatech; L-alpha-amino-omega-benzyl-1-caprolactam) (450 mg, 2.061 mmol) in anhydrous tetrahydrofuran (5 mL) and anhydrous toluene (5 mL) was added DIBAL-H in toluene (20 mL, 20 mmol) slowly at 0° C. The reaction was allowed to warm to room temperature and stirred under nitrogen over the weekend. The reaction was cooled to 0° C. then water and 1N NaOH were added with stirring. The resulting insoluble gummy white solid was removed by filtration through Celite and washed well with ethyl acetate. The filtrate was partitioned and the organic layer was collected, dried over sodium sulfate, and concentrated under vacuum. The residual yellow oil was purified by HPLC (30×100 mm Waters® Sunfire™ column; 5 micron; 35 mL/minute; 210 nM; 0% to 70% CH₃CN+0.05% TFA/water+0.05% TFA over 14 minutes; desired product elutes at 25% CH₃CN+0.05% TFA/water+0.05% TFA). Fractions containing the desired product were lyophilized to give the title compound as a yellow oil (484 mg, 115%—not pure).

### Preparative Example 6

### 1-[({[(3S)-2-oxoazepan-3-yl]amino}carbonyl)oxy]pyrrolidine-2,5-dione

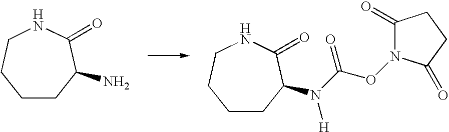

A solution of (3S)-3-amino-1-benzylazepan-2-one (1.0 g, 7.8 mmol; Astatech; L-alpha-amino-omega-benzyl-1-caprolactam) and N,N′-disuccinimidyl carbonate (2.2 g, 8.59 mmol) in anhydrous acetonitrile (10 mL) was stirred at room temperature overnight. The reaction mixture was concentrated under vacuum to give a pale yellow solid which was triturated with ether (3×) to afford the title compound as a solid 1.77 g (84%). LC-MS indicated mostly product plus a small amount of starting material.

### Preparative Example 7

### 1,4-Dibenzyl-1,4-diazepan-6-amine

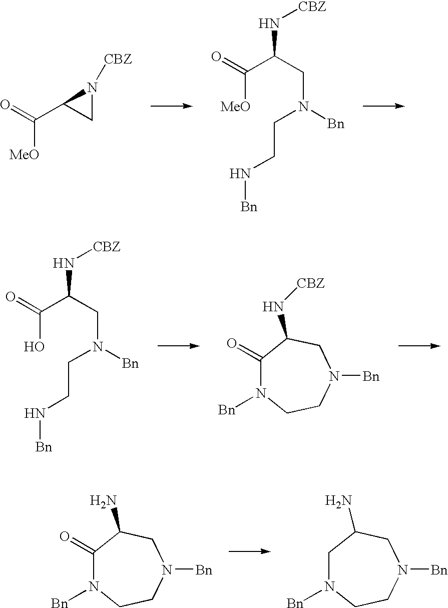

### Step 1: Methyl 3-{benzyl[2-(benzylamino)ethyl]amino}-N-[(benzyloxy)carbonyl]-L-alaninate

A mixture of (2S)-1-[(benzyloxy)carbonyl]aziridine-2-carboxylic acid (262 mg, 1.1 mmol; Kato et al., J. Chem. Soc. Perkin. Trans. 1. 1997, 3219) and N,N′-dibenzylethylenediamine (0.26 mL, 1.1 mmol) in anhydrous tetrahydrofuran (3 mL) was stirred at 70° C. for 16 hours then stirred at room temperature for eleven days. The reaction mixture was concentrated under vacuum and the residue was partitioned between chloroform and water. The organic layer was dried over sodium sulfate, filtered, and concentrated under vacuum. The resiude was purified by preparative TLC on silica gel (4× 1000 micron plates; 10% methanol in dichloromethane) to afford the title compound as a yellow oil (334 mg, 63%).

### Step 2: 3-{Benzyl[2-(benzylamino)ethyl]amino}-N-[(benzyloxy)carbonyl]-L-alanine

A mixture of the product of step 1 (334 mg, 0.70 mmol) and 1N sodium hydroxide (0.84 mL, 0.84 mmol) in ethanol (2 mL) was stirred at room temperature overnight. LC/MS showed reaction complete. The ethanol was removed under vacuum and the residue was acidified by addition of 2 N HCl. The mixture was extracted with chloroform and the organic layer was washed with brine, dried over sodium sulfate, and concentrated under vacuum to afford the title compound as a pale yellow solid which was used without purification in the next step.

### Step 3: Benzyl [(6S)-1,4-dibenzyl-5-oxo-1,4-diazepan-6-yl]carbamate

A mixture of the product of step 2, N-[3-(dimethylamino)propyl]-N′-ethylcarbodiimide (137 mg, 0.71 mmol), N-hydroxybenzotriazole (112 mg, 0.73 mmol), and triethylamine (0.25 mL, 1.79 mmol) in dichloroethane (6 mL) was stirred at room temperature overnight. LC/MS showed starting material still present so the reaction mixture was heated to 50° C. under nitrogen for 1 hour. LC/MS showed reaction complete. The reaction mixture was partitioned between chloroform and 2N HCl. The organic layer was washed with brine, dried over sodium sulfate, and concentrated under vacuum. The residue was purified via liquid chromatography using a Combiflash® system (available from Teledyne Isco) (12 g; 30 mL/min, 254 nM, 10% methanol in dichloromethane for 8 column volumes) to afford the title compound as an orange oil (166 mg, 53% over two steps).

### Step 4: (6S)-6-Amino-1,4-dibenzyl-1,4-diazepan-5-one

A mixture of the product of step 3 (166 mg, 0.375 mmol) and 48% HBr (0.722 mL, 6.38 mmol) in acetonitrile (1 mL) was stirred at 60° C. overnight. LC/MS indicated that the reaction was incomplete so additional 48% HBr (0.3 mL) was added and the reaction mixture was heated for another 1.5 hours. LC/MS showed reaction complete. The reaction mixture was concentrated under vacuum and the residue was purified by HPLC (30×100 mm Waters® Sunfire™ column; 5 micron; 35 mL/minute; 210 nM; 0% to 50% acetonitrile+0.05% TFA/water+0.05% TFA over 14 minutes; desired product elutes at 30% acetonitrile+0.05% TFA/water+0.05% TFA). Fractions containing the desired product were lyophilized over the weekend to afford the title compound as a white solid (116 mg, 100%).

### Step 5: 1,4-Dibenzyl-1,4-diazepan-6-amine

To a solution of the product of step 4 (116 mg, 0.375 mmol) in anhydrous tetrahydrofuran (1.0 mL) and anhydrous toluene (3 mL) was added DIBAL-H in toluene (3.6 mL, 3.60 mmol) slowly at 0° C. under nitrogen. The reaction was allowed to warm to room temperature and stirred for 6 h. LC/MS showed reaction to be complete. The reaction mixture was cooled to 0° C. and water was added followed by slow addition of 1 N NaOH until gas evolution ceased. The resulting slurry was filtered through Celite to remove insoluble solids and washed well with ethyl acetate. The filtrate was partitioned, and the organic layer was collected, dried over sodium sulfate, and concentrated under vacuum. The resulting yellow oil was purified by HPLC (30×100 mm Waters® Sunfire™ column; 5 micron; 35 mL/minute; 210 nM; 0% to 50% acetonitrile+0.05% TFA/water+0.05% TFA over 15 minutes; title compound elutes at 25% CH₃CN+0.05% TFA/water+0.05% TFA) to afford the title compound as a yellow sticky solid (82 mg, 74%).

### Preparative Example 8

### tert-Butyl (2-hydroxyethyl)(4-methoxybenzyl)carbamate

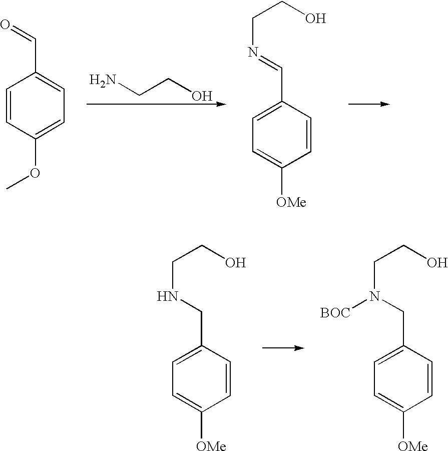

### Step 1: 2-{[(1E)-(4-Methoxyphenyl)methylene]amino}ethanol

Ethanolamine (0.495 mL, 8.19 mmol) and p-anisaldehyde (0.996 mL, 8.19 mmol) were combined (exothermic reaction) and microwaved at 150° C. for 5 minutes then at 160° C. for 5 minutes then at 180° C. for 10 minutes then at 180° C. for 2×20 minutes. NMR showed reaction mostly complete. The reaction mixture was partitioned between ethyl acetate and water. The organic layer was dried over sodium sulfate, filtered, and concentrated under vacuum to give the title compound as a red/orange oil (1.38 g, 94%) which contained about 20% starting aldehyde. The crude product was used without purification in the next step.

### Step 2: 2-[(4-Methoxybenzyl)amino]ethanol

To a solution of the product of step 1 (1.38 g, 7.69 mmol) in ethanol (20 mL) was added sodium borohydride (0.75 g, 19.8 mmol) at room temperature. The reaction was heated to reflux (80° C.) for 2 hours then poured into ice/water and extracted with dichloromethane (2×). The organic layer was washed with brine, dried over sodium sulfate, filtered, and concentrated under vacuum to afford the title compound as a yellow oil contaminated with p-methoxybenzyl alcohol resulting from reduction of the p-anisaldehyde in the starting material. The crude product was used without purification in the next step.

### Step 3: tert-Butyl (2-hydroxyethyl)(4-methoxybenzyl)carbamate

To a solution of the product of step 2 (theoretical amount 1.394 g, 7.69 mmol) in chloroform (10 mL) was added dropwise a solution of di-tert-butyl dicarbonate (1.793 mL, 7.72 mmol) in chloroform (10 mL) at 0° C. The reaction was allowed to warm to room temperature and stirred overnight. The reaction was partitioned between water and dichloromethane. The organic layer was washed with water and brine, dried over sodium sulfate, filtered, and concentrated under vacuum to afford the product as a pale yellow oil (1.68 g, 78%).

### Preparative Example 9

### (6R)-4-(4-Methoxybenzyl)-1,4-oxazepan-6-amine

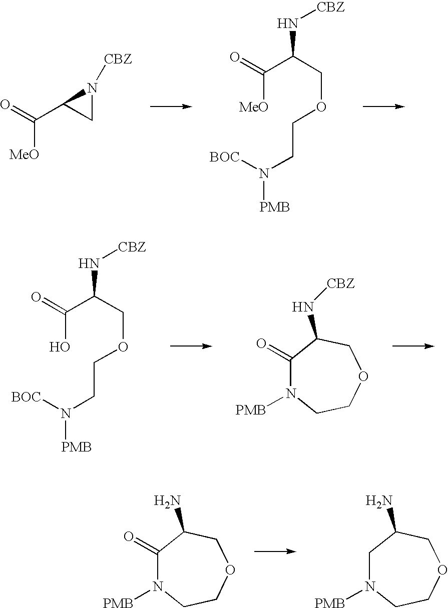

### Step 1: Methyl N-[(benzyloxy)carbonyl]-O-{2-[(tert-butoxycarbonyl)(4-methoxybenzyl)amino]ethyl}-L-serinate

To a solution of (2S)-1-[(benzyloxy)carbonyl]aziridine-2-carboxylic acid (292 mg, 1.24 mmol; Kato et al., J. Chem. Soc. Perkin. Trans. 1 1997, 3219) and tert-Butyl (2-hydroxyethyl)(4-methoxybenzyl)carbamate (0.3728 g, 1.325 mmol) in chloroform (3 mL) was added dropwise boron trifluoride etherate (0.016 mL, 0.124 mmol) at 0° C. The resulting yellow reaction mixture was stirred room temperature overnight. The reaction was parititioned between dichloromethane and saturated sodium bicarbonate. The organic layer was washed with brine, dried over sodium sulfate, filtered, and concentrated under vacuum to give a colorless oil. The crude product was purified by Isco Combiflash (12 g silica gel, 30 mL/min, 254 nM, 0% to 100% ethyl acetate/hexane over 22 column volumes; title compound elutes at 52% ethyl acetate/hexane) to give the title compound as a colorless oil (196 mg, 38%).

### Step 2: N-[(Benzyloxy)carbonyl]-O-{2-[(tert-butoxycarbonyl)(4-methoxybenzyl)amino]ethyl}-L-serine

A mixture of the product of step 1 (196 mg, 0.379 mmol) and 1N sodium hydroxide (0.455 mL, 0.455 mmol) in ethanol (3 mL) was stirred at room temperature for 2 hours. The ethanol was removed under vacuum and the residue was acidified by 2N HCl and extracted with chloroform. The organic layer was washed with brine, dried over sodium sulfate, filtered, and concentrated under vacuum to afford the title comopund as a pale yellow oil which was used without purification in the next step.

### Step 3: Benzyl [(6S)-4-(4-methoxybenzyl)-5-oxo-1,4-oxazepan-6-yl]carbamate

A mixture of the product of step 2, N-[3-(dimethylamino)propyl]-N′-ethylcarbodiimide (92 mg, 0.48 mmol), N-hydroxybenzotriazole (73 mg, 0.48 mmol), and triethylamine (0.16 mL, 1.18 mmol) in dichloromethane (3 mL) was stirred at room temperature overnight. At this point, it was realized that the BOC removal step had been omitted so trifluoroacetic acid (1 mL) was added and the reaction mixture was stirred at room temperature for 90 minutes. BOC removal was complete by LC-MS analysis. The reaction was concentrated under vacuum and purified by HPLC (30×100 mm Waters® Sunfire™ column; 5 micron; 35 mL/minute; 210 nM; 0% to 50% CH₃CN+0.05% TFA/water+0.05% TFA over 15 minutes; recovered SM elutes at 35% CH₃CN+0.05% TFA/water+0.05% TFA). Fractions containing the deprotected starting material were oncentrated and extracted with ethyl acetate (3×). The combined extracts were washed with brine, dried over sodium sulfate, filtered, and concentrated under vacuum to afford the deprotected starting material as a colorless oil (177 mg). A mixture of the deprotected starting material (177 mg), N-[3-(dimethylamino)propyl]-N′-ethylcarbodiimide (92 mg, 0.48 mmol), N-hydroxybenzotriazole (73 mg, 0.48 mmol), and triethylamine (0.16 mL, 1.18 mmol) in dichloromethane (3 mL) was stirred at room temperature overnight then partitioned between chloroform and 2N HCl. The organic layer dried over sodium sulfate, filtered, and concentrated under vacuum. The residue was purified by HPLC (30×100 mm Waters® Sunfire™ column; 5 micron; 35 mL/minute; 210 nM; 10% to 100% CH₃CN+0.05% TFA/water+0.05% TFA over 14 minutes; title compound elutes at 80%CH₃CN+0.05% TFA/water+0.05% TFA). The fractions containing the title compound were collected, concentrated underfvacuumto remove acetonitrile and the remaining aqueous layer was extracted with ethyl acetate (2×). The combined organic layers were washed with brine, dried over sodium sulfate, filtered, and concentrated under vacuum to afford the impure title compound as a yellow oil (287 mg, 170%) which was used without further purification in the next step.

### Step 4: (6S)-6-Amino-4-(4-methoxybenzyl)-1,4-oxazepan-5-one

The product of step 3 (287 mg; note: theoretical amount of starting material present is 169 mg=0.439 mmol) and 48% HBr (1.5 mL, 13.3 mmol) were combined and heated to 60° C. for 1 hour. Acetonitrile (1.0 mL) was added to make the reaction homogeneous. The reaction mixture was heated for another 30 minutes at which point LC/MS showed reaction complete. The reaction mixture was concentrated under vacuum and the residue was purified by HPLC (30×100 mm Waters® Sunfire™ column; 5 micron; 35 mL/minute; 210 nM; 0% to 50% CH₃CN+0.05% TFA/water+0.05% TFA over 14 minutes; title compound elutes at 25% CH₃CN+0.05% TFA/water+0.05% TFA) to afford the title compound (43.7 mg, 39% based on theoretical amount of starting material).

### Step 5: (6R)-4-(4-Methoxybenzyl)-1,4-oxazepan-6-amine

To a solution of the product of step 4 (43.7 mg, 0.175 mmol) in anhydrous tetrahydrofuran (3 mL) and anhydrous toluene (3 mL) was added DIBAL-H in toluene (1.694 mL, 1.694 mmol) slowly at 0° C. under nitrogen. The reaction was allowed to warm to room temperature and stirred overnight. The reaction mixture was cooled to 0° C. as water and 1 N NaOH were added with stirring. The insoluble white gummy solid was removed by filtration through celite and washed well with ethyl acetate. The filtrate was partitioned, and the organic layer was collected, dried over sodium sulfate, and concentrated under vacuum. The resulting yellow oil was purified by HPLC (30×100 mm Waters® Sunfire™ column; 5 micron; 35 mL/minute; 210 mM; 0% to 50% CH₃CN+0.05% TFA/water+0.05% TFA over 15 minutes; title compound elutes at 15% CH₃CN+0.05% TFA/water+0.05% TFA) to afford the title compound as an orange oil (16.5 mg, 40%).

### Preparative Example 10

### (6S)-4-(4-Methoxybenzyl)-1,4-oxazepan-6-amine

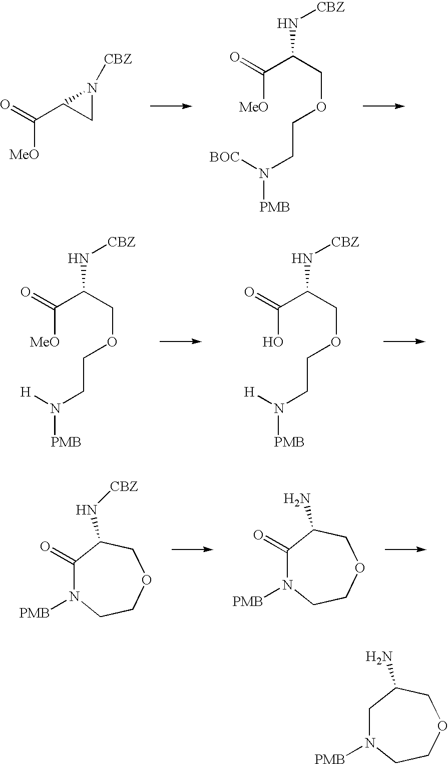

### Step 1: Methyl N-[(benzyloxy)carbonyl]-O-{2-[(tert-butoxycarbonyl)(4-methoxybenzyl)amino]ethyl}-D-serinate

A solution of tert-Butyl (2-hydroxyethyl)(4-methoxybenzyl)carbamate (191 mg, 0.76 mmol) in chloroform (1 mL) was added to a solution of (2R)-1-[(benzyloxy)carbonyl]aziridine-2-carboxylic acid (162 mg, 0.69 mmol; Kato et al., J. Chem. Soc. Perkin. Trans. 1 1997, 3219) in chloroform (3 mL) at 0° C. under nitrogen followed by dropwise addition of boron trifluoride etherate (0.017 mL, 0.138 mmol). The reaction mixture was allowed to warm slowly to room temperature and was stirred at room temperature overnight. The reaction mixture was parititioned between dichloromethane and saturated sodium bicarbonate. The organic layer was washed with brine, dried over sodium sulfate, filtered, and concentrated under vacuum to give a colorless oil. The crude product was purified by Isco Combiflash (12 g silica gel, 30 mL/min, 254 nM, 0% to 100% ethyl acetate/hexane over 15 minutes; title compound elutes at 35% ethyl acetate/hexane) to give the title compound as a colorless oil (190 mg, 57%).

### Step 2: Methyl N-[(benzyloxy)carbonyl]-O-{2-[(4-methoxybenzyl)amino]ethyl}-D-serinate

Trifluoroacetic acid (0.30 mL, 3.9 mmol) was added to a solution of the product of step 1 (190 mg, 0.39 mmol) in chloroform (2 mL) and the resulting yellow solution was stirred at room temperature for 1 hour. The reaction mixture was concentrated under vacuum and the residue was partitioned between ethyl acetate and saturated sodium bicarbonate. The organic layer was washed with brine, dried over sodium sulfate, filtered, and concentrated under vacuum to give the title compound as a colorless oil which was used without purification in the next step.

### Step 3: N-[(Benzyloxy)carbonyl]-O-{2-[(4-methoxybenzyl)amino]ethyl}-D-serine

A mixture of the product of step 2 (theoretical amount 151 mg, 0.39 mmol) and 1N sodium hydroxide (0.468 mL, 0.468 mmol) in ethanol (3 mL) was stirred at room temperature for 2 hours then stored at 0° C. overnight. The ethanol was removed under vacuum and the residue was acidified by 2N HCl. The crude product was purified by HPLC (30×100 mm Waters® Sunfire™ column; 5 micron; 35 mL/minute; 210 nM; 10% to 100% acetonitrile+0.05% TFA/water+0.05% TFA over 14 minutes; title compound elutes at 40% acetonitrile+0.05% TFA/water+0.05% TFA). The fractions containing the title compound were collected and lyophilized over the weekend to afford the title compound as a white solid (103 mg, 71%).

### Step 4: Benzyl [(6R)-4-(4-methoxybenzyl)-5-oxo-1,4-oxazepan-6-yl]carbamate

A mixture of the product of step 3 (103 mg, 0.277 mmol), N-[3-(dimethylamino)propyl]-N′-ethylcarbodiimide (55 mg, 0.29 mmol), N-hydroxybenzotriazole (47 mg, 0.31 mmol), and triethylamine (0.097 mL, 0.69 mmol) in dichloroethane (3 mL) was stirred at room temperature overnight then heated at 50° C. for 1 hour. The reaction mixture was partitioned between chloroform and 2N HCl. The organic layer was washed with brine, dried over sodium sulfate, filtered, and concentrated under vacuum. The residue was purified by Isco Combiflash (12 g Supelco MCI Gel CHP20P, 35 mL/min, 210 nM, 0% to 100% methanol/water over 18 minutes; desired product elutes at 100% methanol) to afford the title compound (64.5 mg, 66%).

### Step 5: (6R)-6-Amino-4-(4-methoxybenzyl)-1,4-oxazepan-5-one

The product of step 4 (64.5 mg, 0.182 mmol) and 48% HBr (0.35 mL, 3.09 mmol) were combined and heated to 60° C. for 2 hours at which point LC/MS showed reaction complete. The reaction mixture was concentrated under vacuum and the aqueous residue was purified by HPLC (30×100 mm Waters® Sunfire™ column; 5 micron; 35 mL/minute; 210 nM; 10% to 60% acetonitrile+0.05% TFA/water+0.05% TFA over 14 minutes; title compound elutes at 35% acetonitrile+0.05% TFA/water+0.05% TFA) to afford impure title compound (48 mg, 120%) which was used without further purification in the next step.

### Step 6: (6S)-4-(4-Methoxybenzyl)-1,4-oxazepan-6-amine

To a solution of the product of step 5 (48 mg, note: theoretical amount of starting material present is 40 mg=0.182 mmol) in anhydrous tetrahydrofuran (1 mL) and anhydrous toluene (3 mL) was added DIBAL-H in toluene (1.694 mL, 1.694 mmol) slowly at 0° C. under nitrogen. The reaction was allowed to warm slowly to room temperature and stirred overnight. The reaction mixture was cooled to 0° C. as water and 1 N NaOH were added with stirring. The insoluble white gummy solid was removed by filtration through celite and washed well with ethyl acetate. The filtrate was concentrated under vacuum and the residue was acidified to pH ˜1 by addition of 2N HCl and purified by HPLC (30×100 mm Waters® Sunfire™ column; 5 micron; 35 mL/minute; 210 nM; 0% to 50% acetonitrile+0.05% TFA/water+0.05% TFA over 15 minutes; title compound elutes at 10% acetonitrile+0.05% TFA/water+0.05% TFA) to afford impure title compound as a yellow oil (16.5 mg). This material was partitioned between ethyl acetate and saturated sodium bicarbonate. The organic layer was washed with brine, dried over sodium sulfate, filtered, and concentrated under vacuum to afford the title compound as a yellow oil. The aqueous layer contained some product by LC-MS and was purified by Isco Combiflash (12 g Supelco MCI Gel CHP20P, 25 mL/min, 210 nM, 0% to 100% methanol/water over 18 minutes; title compound elutes at 100% methanol). Fractions containing the title compound were combined with the yellow oil obtained from the organic layer to afford the pure title compound (7.3 mg, 16%).

### Preparative Example 11

### (4S)-1-(2-hydroxyethyl)azepan-4-aminium trifluoroacetate

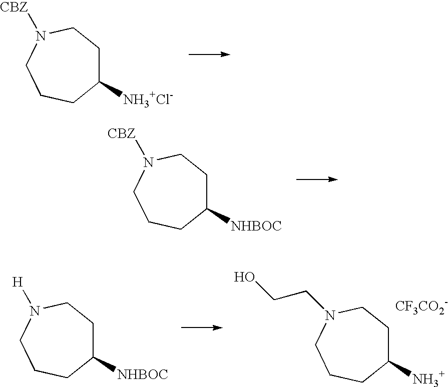

### Step 1: Benzyl (4S)-4-[(tert-butoxycarbonyl)amino]azepane-1-carboxylate

To a solution of benzyl (4S)-4-({[(2,5-dioxopyrrolidin-1-yl)oxy]carbonyl}amino)-azepane-1-carboxylate (63.7 mg, 0.224 mmol) in tetrahydrofuran (1 mL) was added Hunig's Base (0.047 mL, 0.268 mmol), 4-dimethylaminopyridine (6.7 mg, 0.055 mmol), and a solution of di-tert-butyl dicarbonate (0.064 mL, 0.276 mmol) in tetrahydrofuran (0.5 mL) at at room temperature under nitrogen. The reaction was stirred at room temperature overnight then concentrated under vacuum and purified by HPLC (30×100 mm Waters® Sunfire™ column; 5 micron; 35 mL/minute; 210 nM; 10% to 100% acetonitrile+0.05% TFA/water+0.05% TFA over 15 minutes; title compound elutes at 80% acetonitrile+0.05% TFA/water+0.05% TFA). Fractions containing product were combined and lyophilized over the weekend to afford the title comopund as a white solid (63.7 mg, 82%).

### Step 2: tert-Butyl (4S)-azepan-4-ylcarbamate

To a solution of benzyl (4S)-4-[(tert-butoxycarbonyl)arnino]azepane-1-carboxylate (63.7 mg, 0.183 mmol) in methanol (5 mL) was added 20% palladiurn hydroxide on carbon (13.8 mg, 0.020 mmol) and the resulting mixture was subjected to 40 psi of hydrogen in a Parr Shaker overnight. The reaction mixture was filtered through a microfilter which was then washed well with methanol. The filtrate was concentrated under vacuum. The crude title compound thus obtained was used without purification in the next step.

### Step 3: (4S)-1-(2-hydroxyethyl)azepan-4-aminium trifluoroacetate

To a solution of the product of step 2 in dimethylformamide (1 mL) was added (2-bromoethoxy)-tert-butyldimethylsilane (0.051 mL, 0.240 mmol) and Hunig's Base (0.084 mL, 0.480 mmol) at room temperature. The reaction mixture was allowed to stir at room temperature over the weekend then heated to 50° C. for 2 hours. The reaction mixture was partitioned between ethyl acetate and water. The organic layer was washed with water and brine, dried over sodium sulfate, filtered, and concentrated under vacuum to give a yellow oil. The crude intermediate was purified by preparative TLC ((1000 micron; eluted with 10% methanol/dichloromethane; iodine stain) to afford the intermediate (tert-butyl [(4S)-1-(2-{[tert-butyl(dimethyl)silyl]oxy}ethyl)azepan-4-yl]carbamate) as a pale yellow oil (47.3 mg, 53%). This material was dissolved in dichloromethane (2 mL) and trifluoroacetic acid (1 mL) was added. The reaction was stirred at room temperature overnight then concentrated under vacuum to afford the crude title compound as a pale orange oil (18 mg, 28%) which was used without further purification.

### Preparative Example 12

### (4S)-1-(3-Hydroxypropyl)azepan-4-aminium trifluoroacetate

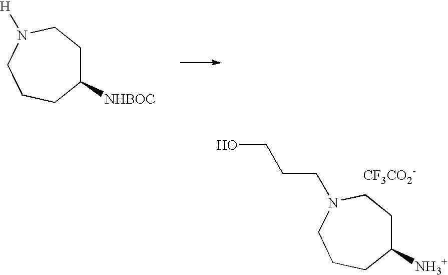

To a solution of tert-Butyl (4S)-azepan-4-ylcarbamate 0028 (108 mg, 0.504 mmol) in anhydrous dimethylformamide (1.5 mL) was added (3-bromopropoxy)-tert-butyldimethylsilane (0.117 mL, 0.504 mmol) and Hunig's Base (0.176 mL, 1.008 mmol) at room temperature. The reaction mixture was allowed to stir at room temperature overnight. LC-MS still showed the presence of starting material so a catalytic amount of sodium iodide was added and the reaction mixture was heated to 50° C. and stirred at 50° C. overnight. The reaction was partitioned between ethyl acetate and 5% sodium thiosulfate. The organic layer was washed with water and brine, dried over sodium sulfate, filtered, and concentrated under vacuum to give a yellow oil. The crude intermediate was purified by plate chrom. (1000 micron; 10% methanol/dichloromethane; iodine stain) to afford the intermediate (tert-butyl [(4S)-1-(3-{[tert-butyl(dimethyl)silyl]oxy}propyl)azepan-4-yl]carbamate) as a yellow oil (94.4 mg, 48%). This material was dissolved in dichloromethane (2 mL) and trifluoroacetic acid (1 mL) was added. The reaction was stirred at room temperature over the weekend then concentrated under vacuum to afford the crude title compound as a pale orange oil (38 mg, 26%) which was azeotroped from toluene and used without further purification.

### Preparative Example 13

### Benzyl (2-{(4S)-4-[(tert-butoxycarbonyl)amino]azepan-1-yl}ethyl)carbamate

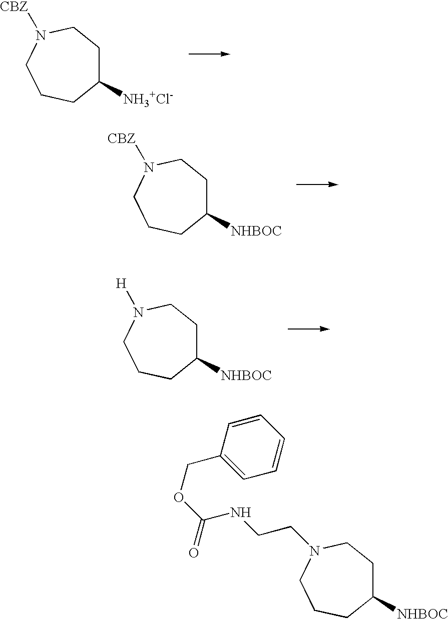

### Step 1: Benzyl (4S)-4-[(tert-butoxycarbonyl)amino]azepane-1-carboxylate

To a solution of benzyl (4S)-4-({[(2,5-dioxopyrrolidin-1-yl)oxy]carbonyl}amino)-azepane-1-carboxylate (920 mg, 3.23 mmol) in anhydrous tetrahydrofuran (1.5 mL) was added Hunig's Base (0.677 mL, 3.88 mmol), 4-dimethylaminopyridine (99 mg, 0.81 mmol), and a solution of di-tert-butyl dicarbonate (0.923 mL, 3.97 mmol) in tetrahydrofuran (0.5 mL) at at room temperature under nitrogen. The reaction was stirred at room temperature overnight then concentrated under vacuum and purified by Isco Combiflash: 35 g of MCI gel CHP20P (Supelco); 35 mL/minute flow rate; 210 nM wavelength; 10% to 100% methanol/water over 10 colume volumes then 100% methanol for 5 column volumens; title compound elutes at 100% methanol. Fractions containing product were combined and lyophilized over the weekend to afford the title comopund as a pale yellow oil (940 mg, 84%) which was used without further purification in the next step.

### Step 2: tert-Butyl (4S)-azepan-4-ylcarbamate

To a solution of the product of step 1 (940 mg, 2.70 mmol) in methanol (20 mL) was added 20% palladium hydroxide on carbon (206 mg, 0.294 mmol) and the resulting mixture was subjected to 40 psi of hydrogen in a Parr Shaker overnight. The reaction mixture was filtered through a microfilter which was then washed well with methanol. The filtrate was concentrated under vacuum to afford the title compound as a colorless foam (592 mg, 102%) which was used without purification in the next step.

### Step 3: Benzyl (2-{(4S)-4-[(tert-butoxycarbonyl)amino]azepan-1-yl}ethyl)carbamate

To a solution of the product of step 2 (102.3 mg, 0.477 mmol) in dimethylformamide (1 mL) was added benzyl (2-iodoethyl)carbamate (149 mg, 0.489 mmol; van Staveren et al. Org. Biomol. Chem. 2004, 2, 2593) and Hunig's Base (0.167 mL, 0.955 mmol) at room temperature. The reaction was heated to 50° C. and stirred at 50° C. under nitrogen overnight. The reaction was partitioned between ethyl acetate and water. The organic layer was washed with water and brine, dried over sodium sulfate, filtered, and concentrated under vacuum to give a yellow oil. The crude product was purified by HPLC (30×100 mm Waters® Sunfire™ column; 5 micron; 35 mL/minute; 210 nM; 10% to 100% acetonitrile+0.05% TFA/water+0.05% TFA over 15 minutes; title compound elutes at 45% acetonitrile+0.05% TFA/water+0.05% TFA). Fractions containing the product were lyophilized overnight to afford the impure title compound as a yellow oil (181 mg, 115%) which was used without further purification.

### Preparative Example 14

### tert-Butyl {(4S)-1-[2-(dimethylamino)ethyl]azepan-4-yl}carbamate

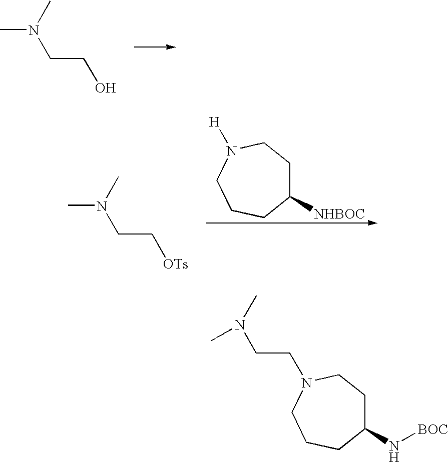

### Step 1: 2-(Dimethylamino)ethyl 4-methylbenzenesulfonate

To a solution of dimethylaminoethanol (342.6 mg, 3.84 mmol) in pyridine (3 mL) was added p-toluenesulfonyl chloride (693 mg, 3.63 mmol) at 0° C. The reaction became bright orange and was stirred at 0° C. for 15 minutes then allowed reaction to warm to room temperature and stirred at room temperature overnight. Ether was then added and the insoluble solids were filtered off. The filtrate was concentrated under vacuum and the residue was purified directly on HPLC (30×100 mm Waters® Sunfire™ column; 5 micron; 35 mL/minute; 210 nM; 0% to 100% acetonitrile+0.05% TFA/water+0.05% TFA over 15 minutes; title compound eluted at 50% acetonitrile+0.05% TFA/water+0.05% TFA) to afford the title compound as a yellow/orange oil. (747 mg, 80%).

### Step 2: tert-Butyl {(4S)-1-[2-(dimethylamino)ethyl]azepan-4-yl}carbamate

To a solution of tert-Butyl (4S)-azepan-4-ylcarbamate (94.9 mg, 0.443 mmol) in acetonitrile (1 mL) was added 2-(dimethylamino)ethyl 4-methylbenzenesulfonate (120.8 mg, 0.496 mmol) and Hunig's Base (0.173 mL, 0.993 mmol) at room temperature. The reaction was heated to 80° C. under nitrogen and stirred at 80° C. overnight. The reaction was filtered to remove insoluble solids and the filtrate was concentrated under vacuum and purified by HPLC (30×100 mm Waters® Sunfire™ column; 5 micron; 35 mL/minute; 210 nM; 0% to 100% acetonitrile+0.05% TFA/water+0.05% TFA over 15 minutes; title compound eluted at 40% acetonitrile+0.05% TFA/water+0.05% TFA). Fractions containing the product were collected and lyophilized overnight to afford the title compound as a yellow oil (104 mg, 73%).

### Preparative Example 15

### 2-Methyl-N-(2,2,7,7-tetramethylazepan-4-yl)propane-2-sulfinamide

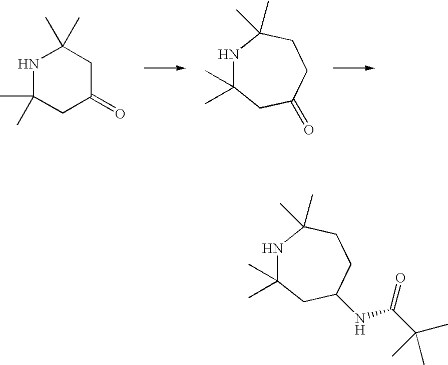

### Step 1: 2,2,7,7-Tetramethylazepan-4-one

To a solution of 2,2,6,6-teramethyl-4-piperidone (620 mg, 4.0 mmol) in anhydrous dichloromethane (6 mL) at −78° C. was added boron trifluoride etherate (1.3 mL, 10.26 mmol) followed by dropwise addition of trimethylsilyldiazomethane in hexane (3.0 mL, 6.0 mmol). The reaction was stirred for 1.5 hours then quenched with saturated sodium bicarbonate and extracted with ethyl acetate. The organic layer was washed with brine, dried over sodium sulfate, filtered, and concentrated under vacuum to afford crude title compound as a yellow oil. LC-MS analysis of the aqueous layers indicated the presence of additional product in the aqueous layer. The aqueous layer was concentrated under vacuum to give a white solid which was redissolved in 1N sodium hydroxide and extracted with ethyl acetate (2×). The organic layer was dried over sodium sulfate, filtered, and concentrated under vacuum to afford additional crude title compound as a yellow oil. Both yellow oil residues were purified by HPLC (30×100 mm Waters® Sunfire™ column; 5 micron; 35 mL/minute; 210 nM; 0% to 100% acetonitrile+0.05% TFA/water+0.05% TFA over 15 minutes; the title compound eluted from 0% to 15% acetonitrile+0.05% TFA/water+0.05% TFA). Fractions containing product were lyophilized overnight to afford the title compound as a white solid (226 mg, 33%).

### Step 2: 2-Methyl-N-(2,2,7,7-tetramethylazepan-4-yl)propane-2-sulfinamide

To a solution of titanium (IV) ethoxide (0.256 mL, 1.236 mmol) and the product of step 1 (101.8 mg, 0.601 mmol) in anhydrous tetrahydrofuran (1.5 mL) was added R-(+)-tert-butanesulfinamide (Acssys Pharmatech) (77.5 mg, 0.639 mmol) under nitrogen and the reaction was heated to 65° C. After 3 hours, the solution was cooled to 0° C. and cannulated into a mixture of sodium borohydride (89.6 mg, 2.368 mmol) in anhydrous tetrahydrofuran (0.5 mL) at −5° C. (ice/brine bath). The resulting yellow solution was stirred for 1.5 hours then quenched with methanol at 0° C. A minimal amount of brine was added and the mixture was stirred vigorously. The the resulting white slurry was filtered through celite and the celite pad well was washed with methanol. The filtrate was collected and concentrated under vacuum. The aqueous residue was purified by Isco Combiflash: 35 g of MCI gel CHP20P (Supelco); 35 mL/minute flow rate; 210 nM wavelength; 0% to 100% acetonitrile/water over 13 colume volumes then 100% acetonitrile for 2 column volumes, ; the title compound eluted at 60-100% acetonitrile/water. Since the product has no UV activity at 210 nM, the fractions were examined by TLC (40: 10:1 chloroform/methanol/conc. ammonium hydroxide) using iodine stain. Fractions containing product were combined and lyophilized overnight to afford the title compound as a white solid (83.4 mg, 50%) which was used without further purification.

### Preparative Example 16

### Benzyl 5-oxoazocane-1-carboxylate and benzyl 4-oxoazocane-1-carboxylate

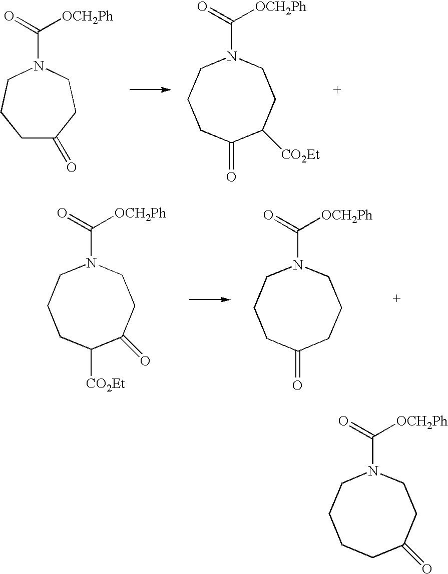

### Step 1: 1-Benzyl 4-ethyl 5-oxoazocane-1,4-dicarboxylate and 1-benzyl 5-ethyl 4-oxoazocane-1,4-dicarboxylate

To a solution of 1-benzyl 5-oxoazepane-1-carboxylate: (1.2 g, 4.86 mmol) in ether (40 mL) at −40° C. was added boron trifluoride etherate (0.734 mL, 5.83 mmol) dropwise under nitrogen followed by dropwise addition of a solution of ethyl diazoacetate (0.604 mL, 5.83 mmol) in ether (10 mL) over 15 minutes. After 1 hour, the reaction was poured into ice/saturated sodium bicarbonate. The organic phase was collected, washed with brine, dried over sodium sulfate, and concentrated under vacuum to afford the title compound as a yellow oil. The crude product was used without purification in the next step.

### Step 2: Benzyl 5-oxoazocane-1-carboxylate and benzyl 4-oxoazocane-1-carboxylate

Potassium carbonate (1.5 g, 10.69 mmol) was added to a solution of the crude product of step 1 in tetrahydrofuran (20 mL) and water (1 mL). The reaction was heated to reflux and refluxed for 2 hours then cooled to 0° C. and diluted with ethyl acetate. The mixture was acidified with stirring to pH 1 by addition of 2N HCl and the layers were separated. The organic layer was washed with brine, dried over sodium sulfate, and concentrated under vacuum to afford an oil which was purified by column chromatography to afford benzyl 5-oxoazocane-1-carboxylate (0.53 g, 42%) and benzyl 4-oxoazocane-1-carboxylate (0.47 g, 37%).

### Preparative Example 17

### Benzyl 5-({[(2,5-dioxopyrrolidin-1-yl)oxy]carbonyl}amino)-azocane-1-carboxylate

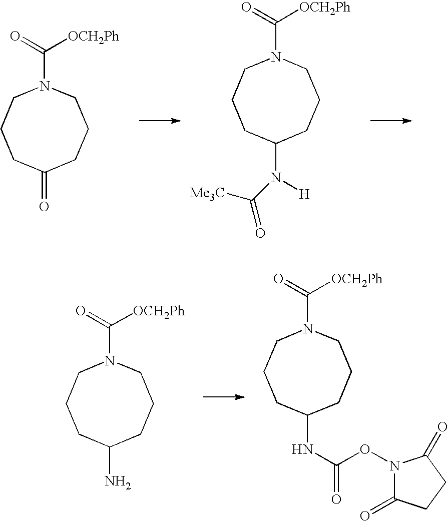

### Step 1: Benzyl 5-[(Tert-Butyl-Sulfinyl)amino]azocane-1-carboxylate

To a solution of titanium (IV) ethoxide (0.684 mL, 3.3 mmol) and benzyl 5-oxoazocane-1-carboxylate (0.43 g, 1.65 mmol) in anhydrous tetrahydrofuran was added tert-butanesulfinamide (0.199 g, 1.65 mmol) under nitrogen and the reaction was heated to 60° C. overnight. The resulting solution was cooled to room temperature then to −5° C. and cannulated into a mixture of sodium borohydride (0.122 g, 3.3 mmol) in anhydrous tetrahydrofuran at −5° C. After 1.5 hours at −5° C. the reaction was quenched with methanol at 0° C. The resulting mixture was poured into brine and stirred vigorously. The resulting mixture was extracted with ethyl acetate and the ethyl acetate layer was washed with brine, dried over magnesium sulfate, filtered, and concentrated under vacuum. The residue was purified by chromatography to give the title compound as an oil which was used directly in the next step.

### Step 2: Benzyl 5-aminoazocane-1-carboxylate

To a solution of the product of step 1 in methanol was added 4N hydrochloric acid in dioxane (0.41 mL, 1.65 mmol). The mixture was stirred for 60 minutes at room temperature then concentrated under vacuum. The residue was triturated with ether and dried under vacuum to afford the title compound which was used in the next step without further purification.

### Step 3: Benzyl 5-({[(2,5-dioxopyrrolidin-1-yl)oxy]carbonyl}amino)-azocane-1-carboxylate

To a solution of the product of step 2 in acetonitrile was added triethylamine (0.27 mL, 1.98 mmol) followed by N-N′-disuccinimidyl carbonate (0.507 g, 1.98 mmol) at room temperature under nitrogen. After 1 hour, the reaction mixture was concentrated under vacuum and purified by reverse-phase HPLC to afford the title compound as a white solid (0.36 g) after lyophilization.

### Preparative Example 18

### tert-Butyl 4-{[(R)-tert-butylsulfinyl]amino}azocane-1-carboxylate

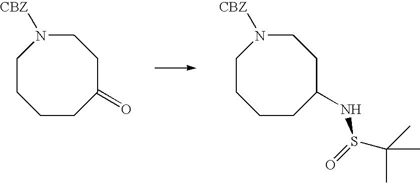

To a solution of titanium (IV) ethoxide (0.16 mL, 0.76 mmol) and tert-butyl 4-oxoazocane-1-carboxylate (100 mg, 0.38 mmol) in anhydrous tetrahydrofuran (3 mL) was added R-(+)-tert-butanesulfinamide (Acssys Pharmatech) (47 mg, 0.38 mmol) under nitrogen and the reaction was heated to 60° C. and stirred at 60° C. overnight. The reaction mixture was cooled to 0° C. and added dropwise to a mixture of sodium borohydride (28 mg, 0.76 mmol) in anhydrous tetrahydrofuran (0.5 mL) at −5° C. (ice/brine bath). The resulting mixture was stirred at room temperature for 1.5 hours then quenched with methanol at 0° C. and poured into brine and extracted with ethyl acetate. The organic layer was concentrated under vacuum and the residue was purified by Isco Combiflash. The product thus obtained was further purified by HPLC (Sunfire column) to afford two diastereomers of the title compound: Isomer A (faster eluting, 20 mg) and Isomer B (slower eluting, 48 mg).

### Preparative Example 19

### Benzyl 5-{[(R)-tert-butylsulfinyl]amino}azonane-1-carboxylate

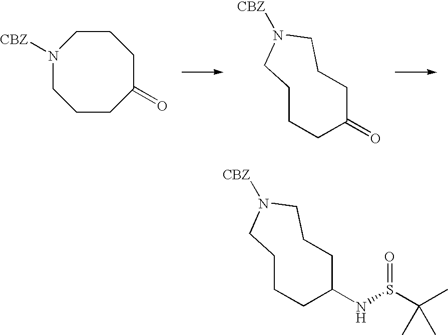

### Step 1: Benzyl 5-oxoazonane-1-carboxylate

To a solution of benzyl 5-oxoazocane-1-carboxylate (760 mg, 2.9 mmol) in ether at −40° C. was added boron trifluoride etherate (0.44 mL, 3.5 mmol) dropwise under nitrogen followed by dropwise addition of a solution of ethyl diazoacetate (0.363 mL, 3.5 mmol) in ether. The reaction mixture was stirred at −40° C. for 1 hour then allowed to warm to 0° C. and stirred at 0° C. for 3 hours. Additional boron trifluoride etherate (0.40 mL, 3.2 mmol) and ethyl diazoacetate (0.33 mL, 3.25 mmol) were added and the resulting mixture was stirred at room temperature overnight. The reaction mixture was poured into ice/saturated sodium bicarbonate. The organic phase was collected, washed with brine, dried over magnesium sulfate, and concentrated under vacuum. The residue was dissolved in a tetrahyrofuran, water, dimethylformamide mixture and potassium carbonate (800 mg, 5.8 mmol) was added. The resulting mixture was refluxed overnight then purified by ISCO Combiflash chromatography to afford the title compound (160 mg, 20%). The crude product was used without purification in the next step.

### Step 2: Benzyl 5-{[(R)-tert-butylsulfinyl]amino}azonane-1-carboxylate

To a solution of titanium (IV) ethoxide (0.13 mL, 0.64 mmol) and the product of step 1 (160 mg, 0.58 mmol) in anhydrous tetrahydrofuran (2 mL) was added R-(+)-tert-butanesulfinamide (Acssys Pharmatech) (70 mg, 0.58 mmol) under nitrogen. The reaction was heated to 60° C. and stirred at 60° C. overnight. Additional titanium (IV) ethoxide (0.13 mL, 0.64 mmol) was added and the reaction stirred at 65° C. for 2.5 hours. The reaction mixture was cooled to 0° C. and added dropwise to a mixture of sodium borohydride (28 mg, 0.76 mmol) in anhydrous tetrahydrofuran (0.5 mL) at −5° C. (ice/brine bath). The resulting mixture was stirred at room temperature for 1.5 hours then quenched with methanol at 0° C. and poured into brine and extracted with ethyl acetate. The organic layer was concentrated under vacuum and the residue was purified by HPLC (Sunfire column) to afford two diastereomers of the title compound: Isomer A (slower eluting, 32 mg) and Isomer B (faster eluting, 45 mg).

### Example 1

### (1S,5R)-2-{[(4S)-azepan-4-ylamino]carbonyl}-7-oxo-2,6-diazabicyclo-[3.2.0-heptane-6-sulfonic acid

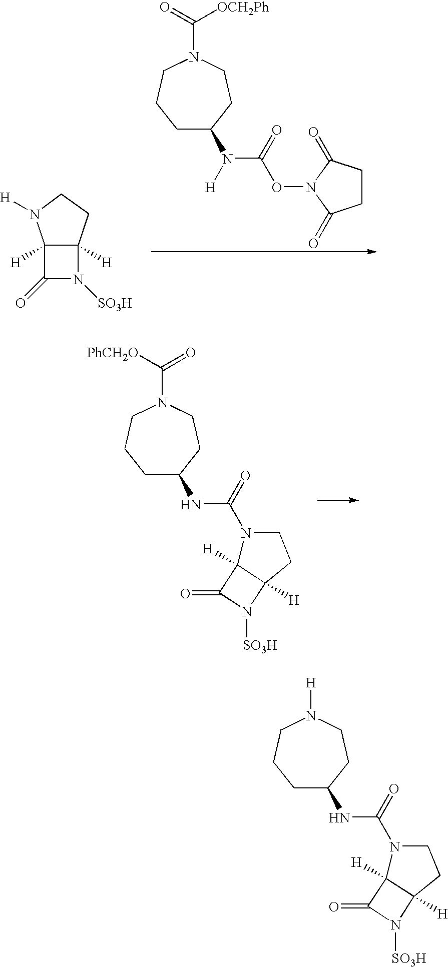

### Step 1: (1S,5R)-2-[({(4S)-1-[(Benzyloxy)carbonyl]azepan-4-yl}amino)carbonyl]-7-oxo-2,6-diazabicyclo[3.2.0]heptane-6-sulfonic acid

To a solution of (1S,5R)-7-oxo-2,6-diazabicyclo[3.2.0]heptane-6-sulfonic acid (0.13 g, 0.68 mmol) and benzyl (4S)-4-({[(2,5-dioxopyrrolidin-1-yl)oxy]-carbonyl}amino)-azepane-1-carboxylate (0.22 g, 0.56 mmol) in acetonitrile (2 mL) was added a solution of sodium bicarbonate (0.11 g, 1.25 mmol) in water (1 mL). After 4 hours, the reaction mixture was purified by reverse-phase HPLC to afford the title compound as a white solid (0.2 g, 76%) after lyophilization.

### Step 2: (1S,5R)-2-{[(4S)-azepan-4-ylamino]carbonyl}-7-oxo-2,6-diazabicyclo-[3.2.0]-heptane-6-sulfonic acid

A solution of (1S,5R)-2-[({(4S)-1-[(benzyloxy)carbonyl]azepan-4-yl}amino)carbonyl]-7-oxo-2,6-diazabicyclo[3.2.0]heptane-6-sulfonic acid (0.2 g, 0.43 mmol) in ethanol (20 mL) and water (2 mL) was hydrogenated at 10 bar over palladium hydroxide using the H-Cube™ continuous flow hydrogenation reactor (ThalesNano, Budapest, Hungary). After the reaction was judged complete by LC-MS, the reaction mixture was concentrated under vacuum and the residue was purified by reverse phase HPLC to afford the title compound as a white solid (0.039 g, 27%) after lyophilization. ¹H NMR (600 MHz, D₂O) δ ppm 5.21 (1H, d, J=4 Hz), 4.74 (1H, dd, J=4 Hz), 3.91 (1H, dd, J=10, 9 Hz), 3.80-3.88 (1H, m), 3.28-3.44 (3H, m), 3.14-3.22 (2H, m), 2.37 (1H, dd, J=14, 6 Hz), 2.12-2.18 (1H, m), 2.04-2.11 (1H, m), 1.85-2.00 (3H, m), 1.74-1.84 (1H, m), 1.58-1.68 (1H, m). ¹³C NMR (125 MHz, D₂O) δ ppm 164.8, 157.1, 66.8, 61.2, 50.5, 46.0, 44.2, 42.1, 33.0, 31.1, 26.8, 21.0. LC-MS (neg. ionization) m/e 331 (M−H).

### Example 2

### (1S,5R)-2-{[(4R)-Azepan-4-ylamino]carbonyl}-7-oxo-2,6-diazabicyclo-[3.2.0-heptane-6-sulfonic acid

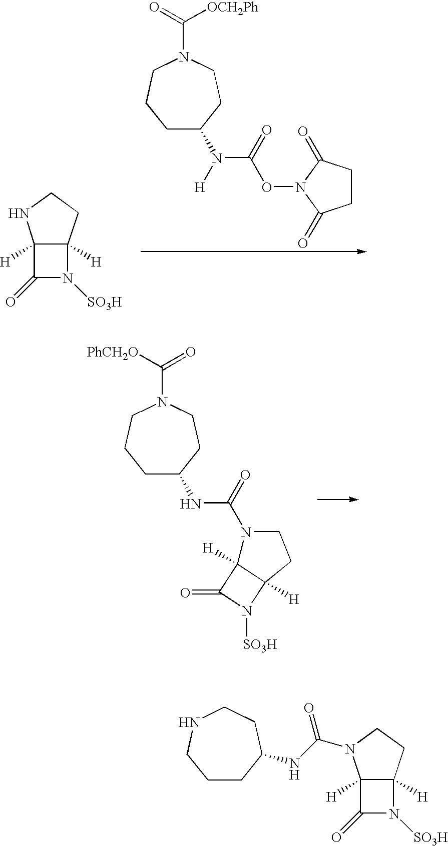

Using the procedure outlined in Example 1 for the diastereomeric product, the title compound was obtained as a white solid after lyophilization. ¹H NMR (500 MHz, D₂O) δ ppm 5.23 (1H, d, J=4 Hz), 4.74 (1H, dd, J=4 Hz), 3.91 (1H, dd, J=10, 9 Hz), 3.80-3.88 (1H, m), 3.28-3.44 (3H, m), 3.14-3.22 (2H, m), 2.39 (1H, dd, J=14, 6 Hz), 2.12-2.18 (1H, m), 2.04-2.11 (1H, m), 1.84-2.00 (3H, m), 1.74-1.84 (1H, m), 1.58-1.68 (1H, m). LC-MS (neg. ionization) m/e 331 (M−H).

### Example 3

### (1S,5R)-2-{[(Cycloheptylamino]carbonyl}-7-oxo-2,6-diazabicyclo-[3.2.0]-heptane-6-sulfonic acid

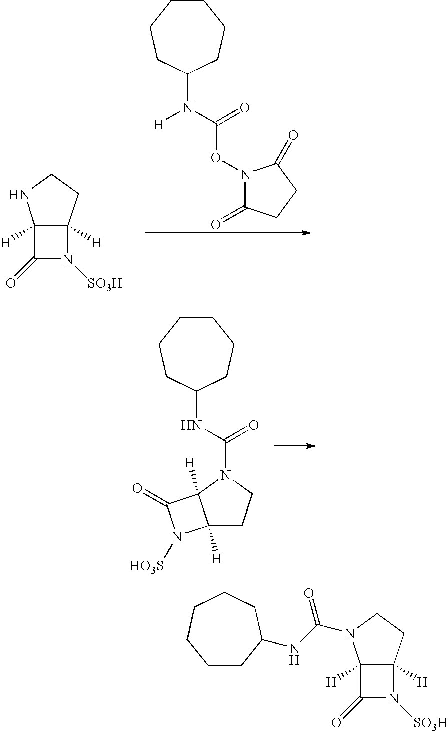

Using the procedure outlined in Example 1, the title compound was obtained as a white solid after lyophilization. ¹H NMR (500 MHz, D₂O) δ ppm 5.23 (1H, d, J=4 Hz), note: the anticipated signal at ˜4.74 was obscured by a large H₂O peak and was not observed in this spectrum), 3.90 (1H, dd, J=11, 11 Hz), 3.62-3.70 (1H, m), 3.30 (1H, ddd, J=11, 11, 6 Hz), 2.40 (1H, dd, J=14, 6 Hz), 1.80-1.95 (3H, m), 1.40-1.65 (10H, m).

### Example 4

### (1S,5R)-2-{[(3S)-Azepan-3-ylamino]carbonyl}-7-oxo-2,6-diazabicyclo[3.2.0]-heptane-6-sulfonic acid

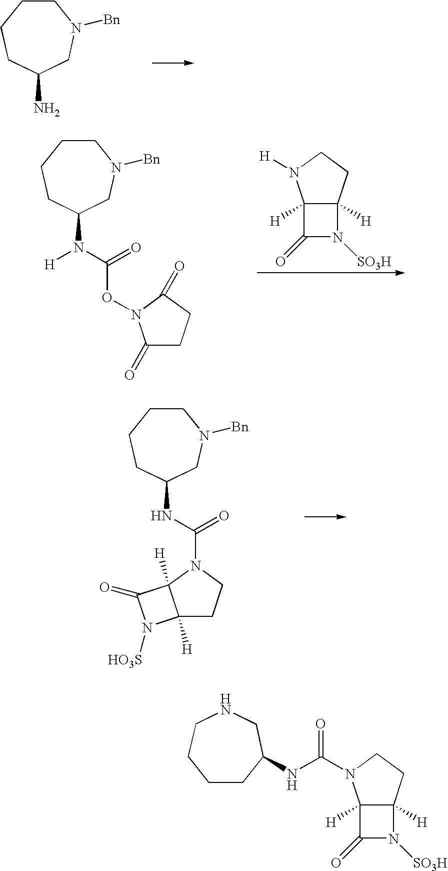

### Step 1: 1-[({[(3S)-1-Benzylazepan-3-yl]amino}carbonyl)oxy]pyrrolidine-2,5-dione

To a solution of N,N′-disuccinimidyl carbonate (129 mg, 0.505 mmol) in acetonitrile (1 mL) was added a solution of (3S)-1-benzylazepan-3-amine (97 mg, 0.475 mmol) in acetonitrile (2 mL) followed by diisopropyl-ethylamine (0.085 mL, 0.487 mmol) and the resulting solution was stirred overnight at room temperature under nitrogen. The reaction mixture was then concentrated under vacuum and the residue was triturated with ether. The ether layer was decanted off and the insoluble oil was dried under vacuum to afford the title compound which was used without purification.

### Step 2: (1S,5R)-2-({[(3 S)-1-Benzylazepan-3-yl]amino}carbonyl)-7-oxo-2,6-diazabicyclo[3.2.0]heptane-6-sulfonic acid

To a mixture of (1S,5R)-7-oxo-2,6-diazabicyclo[3.2.0]heptane-6-sulfonic acid (92.6 mg, 0.482 mmol) and 1-[({[(3S)-1-benzylazepan-3-yl]amino}carbonyl)oxy]pyrrolidine-2,5-dione (164 mg, 0.475 mmol) in acetonitrile (0.5 mL) was added a solution of sodium bicarbonate (63 mg, 0.750 mmol) in water (0.5 mL). The resulting solution was allowed to stir at room temperature overnight then concentrated under vacuum to remove acetonitrile. The residue was purified by HPLC (30×100 mm Waters® Sunfire™ column; 5 micron; 35 mL/minute; 210 nM; 0% to 50% CH₃CN+0.05% TFA/water+0.05% TFA over 14 minutes; product elutes at 25% CH₃CN+0.05% TFA/water+0.05% TFA). Fractions containing the desired product were lyophilized overnight to give the title compound as a white solid (72 mg, 36%).

### Step 3: (1S,5R)-2-{[(3S)-Azepan-3-ylamino]carbonyl}-7-oxo-2,6-diazabicyclo[3.2.0]-heptane-6-sulfonic acid

The product of step 2 (30 mg, 0.071 mmol) and palladum hydroxide (8.9 mg, 0.013 mmol) were combined and hydrogenated under 40 psi of hydrogen in a Parr Shaker overnight. The reaction was filtered through a microfilter and the collected solid was washed well with Methanol and water. The filtrate was concentrated under vacuum and purified by HPLC (250×21.2 mm Phenomenex Synergi Polar-RP 80A column; 10 micron; 35 mL/minute; 210 nM; 0% to 70% Methanol/water over 14 minutes; product elutes at 25% Methanol/water). Fractions containing the desired product were lyophilized overnight to give the title compound as a white solid (9.3 mg, 39%). ¹H NMR (500 MHz, D₂O) δ ppm 5.23 (1H, d, J=4 Hz), (note: the anticipated signal at ˜4.75 ppm was obscured by large H₂O peak), 4.0-4.1 (1H, m), 3.92 (1H, t, J=10 Hz), 3.15-3.4 (5H, m) 2.39 (1H, dd, J=14, 6 Hz), 2.02-2.10 (1H, m), 1.80-2.00 (4H, m), 1.84-2.00 (3H, m), 1.65-1.75 (1H, m), 1.55-1.65 (1H, m). LC-MS MS (neg. ionization) m/e 331 (M−H).

### Example 5

### (1S,5R)-7-Oxo-2-({[(3S)-2-oxoazepan-3-yl]amino}carbonyl)-2,6-diazabicyclo[3.2.0]heptane-6-sulfonic acid

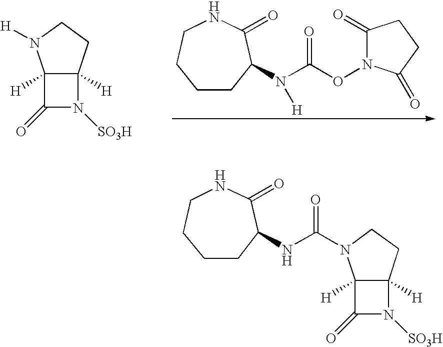

To a mixture of (1S,5R)-7-oxo-2,6-diazabicyclo[3.2.0]heptane-6-sulfonic acid (157.7 mg, 0.821 mmol) and 1-[({[(3S)-1-benzyl-2-oxoazepan-3-yl]amino}carbonyl)oxy]-pyrrolidine-2,5-dione (205 mg, 0.761 mmol) in acetonitrile (3 mL) was added a solution of sodium bicarbonate (128.8 mg, 1.53 mmol) in water (3 mL). The resulting mixture was stirred at room temperature for five hours then stored in the freezer over the weekend. The reaction mixture was then concentrated under vacuum and the residue was purified by HPLC (21.2×250 mm Phenomenex Synergi Polar-RP 80A column; 10 micron; 35 mL/minute; 210 nM; 0% to 10% methanol/water over 9 min; product elutes at 4% methanol/water). Fractions containing the desired product were lyophilized overnight to give the title compound as a white solid (169 mg, 64%). 1H NMR (600 MHz, D₂O) δ ppm 5.26 (1H, d, J=4 Hz), 4.75 (1H, dd, J=5 Hz), 4.49 (1H, dd, J=12 m 1 Hz), 4.00 (1H,dd, J=11, 9 Hz), 3.2-3.4 (3H, m), 2.42 (1H, dd, J=14, 6 Hz), 1.6-2.0 (6H, m), 1.30-1.40 (1H, m). LC-MS (neg. ionization) m/e 345 (M−H).

### Example 6

### (1S,5R)-2-[(1,4-Diazepan-6-ylamino)carbonyl]-7-oxo-2,6-diazabicyclo[3.2.0]heptane-6-sulfonic acid

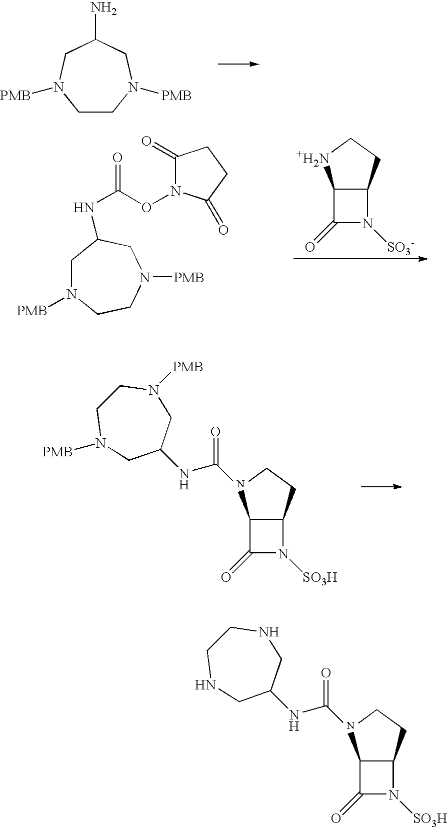

### Step 1: 1-({[(1,4-Dibenzyl-1,4-diazepan-6-yl)amino]carbonyl}oxy)pyrrolidine-2,5-dione

To a solution of N,N′-disuccinimidyl carbonate (78 mg, 0.306 mmol) in acetonitrile (1.5 mL) was added dropwise a solution of 1,4-dibenzyl-1,4-diazepan-6-amine (82.2 mg, 0.278 mmol) and Hunig's Base (0.049 mL, 0.278 mmol) in acetonitrile (1.5 mL) at room temperature under nitrogen. The reaction was allowed to stir at room temperature overnight. The reaction mixture was concentrated under vacuum and the residue was triturated with ether. The ether layer was decanted off and the insoluble oil was dried under vacuum to afford the title compound which was used without purification in the next step.

### Step 2: (1S,5R)-2-{[(1,4-Dibenzyl-1,4-diazepan-6-yl)amino]carbonyl}-7-oxo-2,6-diazabicyclo-[3.2.0]-heptane-6-sulfonic acid

To a solution of 1-({[(1,4-dibenzyl-1,4-diazepan-6-yl)amino]carbonyl}oxy)pyrrolidine-2,5-dione (0.121 g, 0.278 mmol) in acetonitrile (3 mL) was added a solution of (1S,5R)-7-oxo-2,6-diazabicyclo[3.2.0]heptane-6-sulfonic acid (0.0576 g, 0.300 mmol) in water (3 mL) followed by sodium bicarbonate (0.0234 g, 0.279 mmol). The resulting solution was stirred at room temperature overnight then concentrated under vacuum to remove acetonitrile. The resulting aqueous layer was purified by HPLC (30×100 mm Waters® Sunfire™ column; 5 micron; 35 mL/minute; 210 nM; 0% to 30% CH₃CN+0.05% TFA/water+0.05% TFA over 15 minutes; title compound elutes at 27% CH₃CN+0.05% TFA/water+0.05% TFA). The fractions containing pure product were collected and lyophilized over the weekend to give the title compound as a white solid (50 mg, 35%).

### Step 3: (1S,5R)-2-[(1,4-Diazepan-6-ylamino)carbonyl]-7-oxo-2,6-diazabicyclo[3.2.0]heptane-6-sulfonic acid

Palladium hydroxide (13.2 mg of 20% palladium hydroxide on carbon) and acetic acid (0.030 mL) were added to a solution of (1S,5R)-2-{[(1,4-dibenzyl-1,4-diazepan-6-yl)amino]carbonyl}-7-oxo-2,6-diazabicyclo-[3.2.0]-heptane-6-sulfonic acid (50 mg) in methanol (6 mL) and the resulting reaction mixture was hydrogenated under 45 psi of H2 in a Parr Shaker overnight. The reaction was then filtered through a microfilter and the catalyst washed well with methanol and water. The filtrate was concentrated under vacuum and the residue was purified by HPLC (250×21.2 mm Phenomenex Synergi Polar-RP 80A column; 10 micron; 35 mL/minute; 210 nM; 0% to 30% Methanol/water over 15 minutes; title compound elutes at 3% methanol/water) to give the title compound as an off-white (slightly purple) solid (21.7 mg, 67%). 1H NMR (500 MHz, D₂O) δ ppm 5.23 (1H, d, J=4 Hz), (note: the anticipated signal at ˜4.75 ppm was obscured by large H₂O peak), 4.34-4.40 (1H, m), 4.00 (1H, t, J=10 Hz), 3.32-3.63 (9H, m), 2.41 (1H, dd, J=14, 6 Hz), 1.90-1.98 (1H, m). LC-MS (neg. ionization) m/e 332 (M−H).

### Example 7

### (1S,5R)-2-{[(6R)-1,4-Oxazepan-6-ylamino]carbonyl}-7-oxo-2,6-diazabicyclo[3.2.0]heptane-6-sulfonic acid

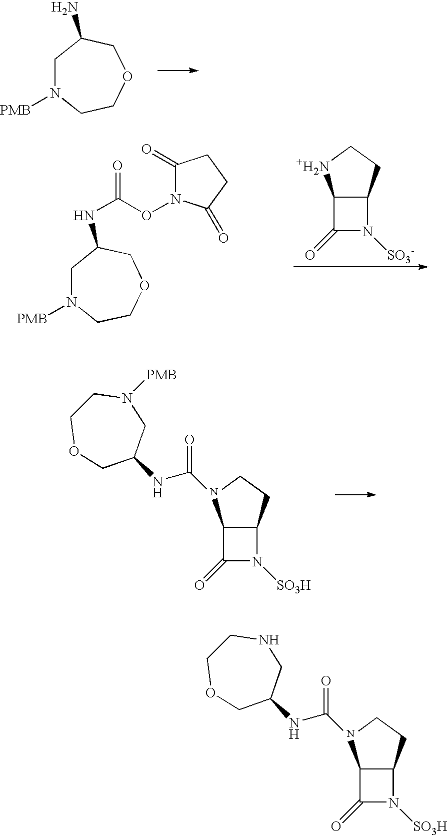

### Step 1: 1-[({[(6R)-4-(4-Methoxybenzyl)-1,4-oxazepan-6-yl]amino}carbonyl)oxy]pyrrolidine-2,5-dione

To a solution of N,N′-disuccinimidyl carbonate (19.0 mg, 0.074 mmol) in acetonitrile (1 mL) was added a solution of (6R)-4-(4-methoxybenzyl)-1,4-oxazepan-6-amine (16.5 mg, 0.070 mmol) in acetonitrile (1 mL, 0.5 mL rinse) followed by Hunig's Base (0.012 mL, 0.070 mmol) at room temperature under nitrogen. The reaction was allowed to stir at room temperature overnight. The reaction was concentrated under vacuum and the residue was triturated with ether. The ether layer was decanted off and the insoluble oil was dried under vacuum to affored the title compound which was used without further purification in the next step.

### Step 2: (1S,5R)-2-({[(6R)-4-(4-Methoxybenzyl)-1,4-oxazepan-6-yl]amino}carbonyl)-7-oxo-2,6-diazabicyclo[3.2.0]heptane-6-sulfonic acid

To a solution of the product of step 1 (26 mg=theoretical yield of step 1, 0.07 mmol;) in acetonitrile (1 mL) was added a solution of (1S,5R)-7-oxo-2,6-diazabicyclo[3.2.0]heptane-6-sulfonic acid (13.8 mg, 0.072 mmol) in water (1 mL) followed by sodium bicarbonate (8.3 mg, 0.099 mmol). The resulting solution was allowed to stir at room temperature overnight. The reaction was concentrated under vacuum to remove acetonitrile. The resulting aqueous layer was purified by HPLC (30×100 mm Waters® Sunfire™ column; 5 micron; 35 mL/minute; 210 nM; 0% to 50% acetonitrile+0.05% TFA/water+0.05% TFA over 15 minutes; desired product elutes at 25% acetonitrile+0.05% TFA/water+0.05% TFA). The fractions were collected and lyophilized to afford the title compound as a white solid (8.3 mg, 26%).

### Step 3: (1S,5R)-2-{[(6R)-1,4-Oxazepan-6-ylamino]carbonyl}-7-oxo-2,6-diazabicyclo[3.2.0]heptane-6-sulfonic acid

A mixture of the product of step 2 (8.3 mg, 0.018 mmol), 20% palladium hydroxide on carbon (3.7 mg), and acetic acid (0.030 mL) in methanol (3 mL) and water (1 mL) was hydrogenated under 40 psi of hydrogen in a Parr Shaker overnight. The reaction was filtered through a microfilter and washed catalyst well with Methanol and water. The filtrate was concentrated under vacuum and purified by HPLC (250×21.2 mm Phenomenex Synergi Polar-RP 80A column; 10 micron; 35 mL/minute; 210 nM; 0% to 30% Methanol/water over 15 minutes; title compound elutes at 5% Methanol/water) to give the title compound as a pale yellow solid (4.8 mg, 79%). 1H NMR (500 MHz, D₂O) δ ppm 5.25 (1H, d, J=4 Hz), (note: the anticipated signal at ˜4.75 ppm was obscured by large H₂O peak), 4.27 (1H, br s), 3.92-4.02 (4H, m), 3.80-3.84 (1H, m), 3.35-3.52 (5H, m), 2.38-2.44 (1H, m), 1.89-1.98 (1H, m). LC-MS (neg. ionization) m/e 333 (M−H).

### Example 8

### (1S,5R)-2-{[(6S)-1,4-Oxazepan-6-ylamino]carbonyl}-7-oxo-2,6-diazabicyclo[3.2.0]heptane-6-sulfonic acid

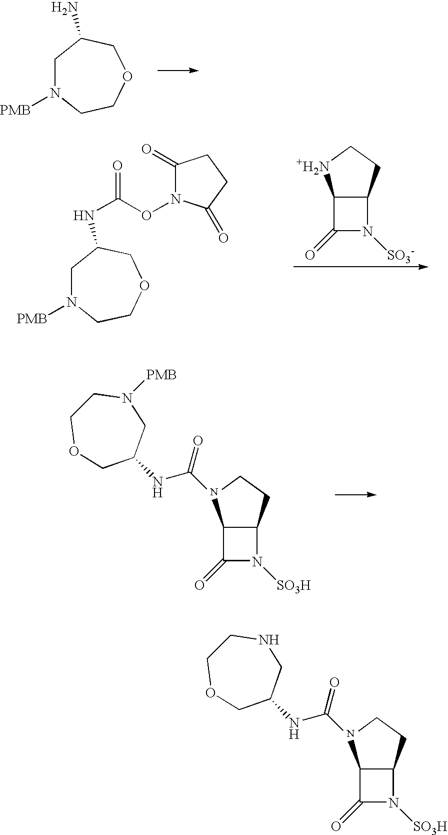

### Step 1: 1-[({[(6S)-4-(4-Methoxybenzyl)-1,4-oxazepan-6-yl]amino}carbonyl)oxy]pyrrolidine-2,5-dione

To a solution of N,N′-disuccinimidyl carbonate (10.9 mg, 0.042 mmol) in acetonitrile (1 mL) was added a solution of (6S)-4-(4-methoxybenzyl)-1,4-oxazepan-6-amine (16.5 mg, 0.070 mmol) in acetonitrile (1 mL, 0.5 mL rinse) at room temperature under nitrogen. The reaction was allowed to stir at room temperature overnight. The reaction was concentrated under vacuum and the residue was triturated with ether. The ether layer was decanted off and the insoluble oil was dried under vacuum to affored the title compound which was used without further purification in the next step.

### Step 2: (1S,5R)-2-({[(6S)-4-(4-Methoxybenzyl)-1,4-oxazepan-6-yl]amino}carbonyl)-7-oxo-2,6-diazabicyclo[3.2.0]heptane-6-sulfonic acid

To a solution of the product of step 1 (12.2 mg=theoretical yield of step 1, 0.035 mmol) in acetonitrile (1 mL) was added a solution of (1S,5R)-7-oxo-2,6-diazabicyclo[3.2.0]heptane-6-sulfonic acid (8 mg, 0.042 mmol) in water (1 mL) followed by sodium bicarbonate (4.9 mg, 0.058 mmol). The resulting solution was allowed to stir at room temperature overnight. The reaction was concentrated under vacuum to remove acetonitrile. The resulting aqueous layer was purified by purified by Isco Combiflash (12 g Supelco MCI Gel CHP20P, 30 mL/min, 210 nM, 100% water for 5 minutes then 0% to 100% methanol/water over 11 minutes; title compound elutes at 55% methanol/water). The fractions containing the title compound were collected and lyophilized to afford the title compound as a white solid (6.9 mg, 46%).

### Step 3: (1S,5R)-2-{[(6S)-1,4-Oxazepan-6-ylamino]carbonyl}-7-oxo-2,6-diazabicyclo[3.2.0]heptane-6-sulfonic acid

A mixture of the product of step 2 (6.9 mg, 0.016 mmol) and 20% palladium hydroxide on carbon (2 mg) in methanol (3 mL) was hydrogenated under 45 psi of hydrogen in a Parr Shaker overnight. The reaction was filtered through a microfilter and washed catalyst well with Methanol and water. The filtrate was concentrated under vacuum and purified by HPLC (250×21.2 mm Phenomenex Synergi Polar-RP 80A column; 4 micron; 5 mL/minute; 210 nM; 0% to 70% methanol/water over 15 minutes; title compound elutes at 10% methanol/water) to give impure title compound as a gummy solid which was triturated with acetonitrile (2×). The insoluble white solid was collected by centrifugation and dried under vacuum to afford the title compound as a pale yellow solid (1.7 mg, 31%) which still contained in impurity by NMR. 1H NMR (500 MHz, D₂O) δ ppm 5.25 (1H, d, J=3 Hz), (note: the anticipated signal at ˜4.75 ppm was obscured by large H₂O peak), 4.28 (1H, br s), 3.92-4.0 (4H, m), 3.81-3.84 (1H, m), 3.36-3.52 (5H, m), 2.39-2.41 (1H, m), 1.89-1.98 (1H, m). LC-MS (neg. ionization) m/e 333 (M−H).

### Example 9

### (1S,5R)-2-({[(4S)-1-Methylazepan-4-yl]amino}carbonyl)-7-oxo-2,6-diazabicyclo[3.2.0]heptane-6-sulfonic acid

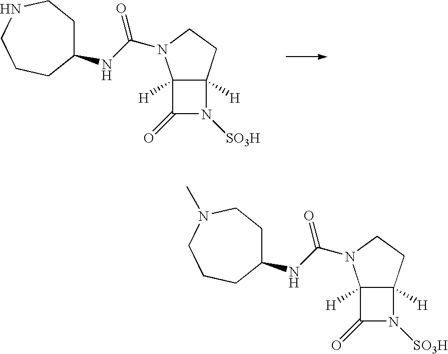

Sodium cyanoborohydride (18.9 mg, 0.30 mmol) was added to a solution of (1S,5R)-2-{[(4S)-azepan-4-ylamino]carbonyl}-7-oxo-2,6-diazabicyclo[3.2.0]heptane-6-sulfonic acid (50 mg, 0.15 mmol) and 37% aqueous formaldehyde (0.056 mL, 0.75 mmol) in acetonitrile at 0° C. The resulting mixture was stirrred for 10 minutes then the pH was adjusted to ˜5-6 by addition of a few drops of acetic acid. After 1 hour at room temperature LC-MS showed reaction completed. The acetonitrile was removed under vacuum and the residue was dissolved in water. The pH was adjusted to ˜7 by addition of saturated sodium bicarbonate and the crude product was purified by HPLC (phenomenex column, methanol/water). The fraction containing the title compound was lyophilized to afford a white solid which was triturated with acetonitrile to afford the impure title compound as a white solid (12 mg, 23%). ¹H NMR (500 MHz, D₂O) δ ppm 5.23 (1H, d, J=4 Hz), 4.74 (1H, dd, J=4 Hz), 3.85-3.96 (2H, m), 3.28-3.48 (5H,m), 2.87 (3H, s), 2.39 (1H, dd, J=14, 6 Hz), 2.10-2.20 (2H, m), 1.82-2.02 (4H, m), 1.60-1.70 (1H, m). LC-MS m/e 369 (M+Na), 347 (M+H).

### Example 10

### (1S,5R)-2-({[(4S)-1,1-Dimethylazepanium-4-yl]amino}carbonyl)-7-oxo-2,6-diazabicyclo[3.2.0]heptane-6-sulfonate

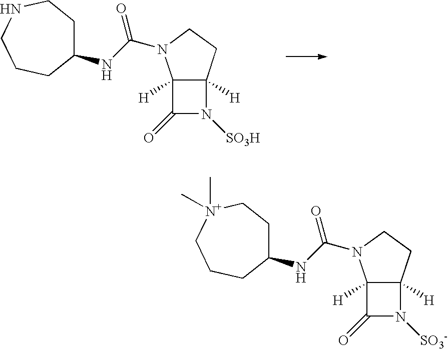

A mixture of (1S,5R)-2-{[(4S)-azepan-4-ylamino]carbonyl}-7-oxo-2,6-diazabicyclo[3.2.0]heptane-6-sulfonic acid (25 mg, 0.075 mmol), iodomethane (0.012 mL, 0.188 mmol), triethylamine (0.031 mL, 0.226 mmol) and 4-dimethylaminopyridine (9.2 mg, 0.075 mmol) in dimethylformamide (10 mL) was stirred at room temperature for 2 hours. Additional iodomethane (0.007 mL, 0.112 mmol) was added and the reaction mixture was stirred at room temperature overnight. The mixture was purified by HPLC to afford the title compound as a white solid (8 mg, 29%). ¹HNMR (600 MHz, D₂O) δ ppm 5.21 (1H, d, J=4 Hz), 4.72 (1H, dd, J=4 Hz), 3.90 (1H, dd, J=10, 9 Hz), 3.75-3.83 (1H, m), 3.50-3.56 (2H, m), 3.36-3.44 (2H, m), 3.28-3.34 (1H, m), 3.09 (3H, s), 3.08 (3H, s), 2.37 (1H, dd, J=10, 6 Hz), 1.80-2.10 (6H, m), 1.50-1.60 (1H, m). LC-MS m/e 361 (M+).

### Example 11

### (1S,5R)-2-({[(4S)-1-(2-Hydroxyethyl)azepan-4-yl]amino}carbonyl)-7-oxo-2,6-diazabicyclo[3.2.0]heptane-6-sulfonic acid

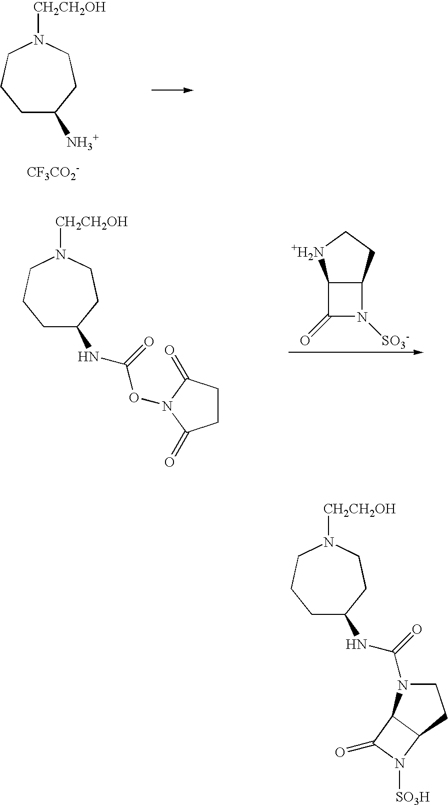

### Step 1: 1-[({[(4S)-1-(2-Hydroxyethyl)azepan-4-yl]amino}carbonyl)oxy]pyrrolidine-2,5-dione

To a solution of 2-[(4S)-4-aminoazepan-1-yl]ethanol (4S)-1-(2-hydroxyethyl)azepan-4-aminium trifluoroacetate (18 mg, 0.066 mmol) ) and triethylamine (0.021 mL, 0.152 mmol) in acetonitrile (2.5 mL) was added N,N′-disuccinimidyl carbonate (0.0363 g, 0.142 mmol) at room temperature. The resulting solution was stirred at room temperature overnight. The reaction was concentrated under vacuum and triturated with hexane then ether (2×) to afford the title compound as an orange oil which was used without purification in the next step.

### Step 2: (1S,5R)-2-({[(4S)-1-(2-Hydroxyethyl)azepan-4-yl]amino}carbonyl)-7-oxo-2,6-diazabicyclo[3.2.0]heptane-6-sulfonic acid

To a solution of the product of step 1 (theoretical amount of starting material present is 19.8 mg, 0.066 mmol) in acetonitrile (1 mL) was added (1S,5R)-7-oxo-2,6-diazabicyclo[3.2.0]heptane-6-sulfonic acid (33.1 mg, 0.172 mmol) followed by a solution of sodium bicarbonate (28.7 mg, 0.342 mmol) in water (1 mL). The reaction was stirred at room temperature overnight. The reaction mixture was concentrated under vacuum and purified by HPLC (21.2×250 mm Phenomenex Synergi Polar-RP 80A column; 10 micron; 35 mL/minute; 210 nM; 0% to 20% methanol/water over 15 minutes; title compound elutes at 8% methanol/water). Fractions containing the desired product were collected and lyophilized over the weekend to afford a solid which was triturated with acetonitrile (2×) to afford the title compound as a white solid (14 mg, 56%). ¹H NMR (500 MHz, D₂O) δ ppm 5.23 (1H, d, J=4 Hz), 4.74 (1H, dd, J=4 Hz), 3.80-3.95 (4H, m), 3.25-3.55 (7H, m), 2.37 (1H, dd, J=14, 6 Hz), 1.80-2.20 (6H, m), 1.58-1.68 (1H, m). LC-MS (neg. ionization) m/e 375 (M−H).

### Example 12

### (1S,5R)-2-({[(4S)-1-(3-Hydroxypropyl)azepan-4-yl]amino}carbonyl)-7-oxo-2,6-diazabicyclo-[3.2.0]heptane-6-sulfonic acid

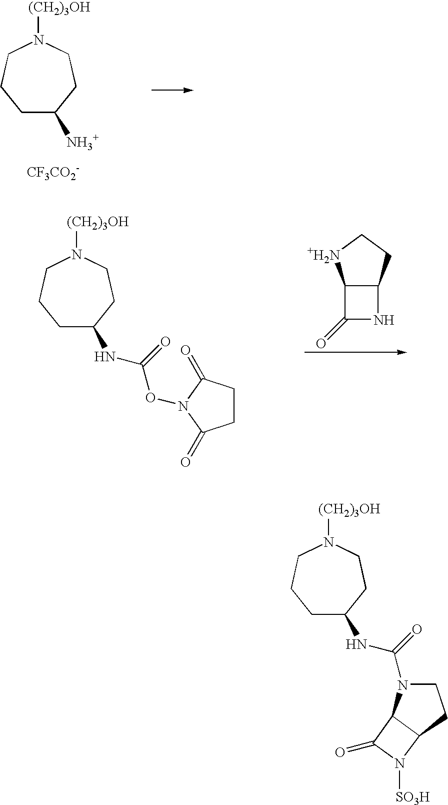

### Step 1: 1-[({[(4S)-1-(3-Hydroxypropyl)azepan-4-yl]amino }carbonyl)oxy]pyrrolidine-2,5-dione

To a solution of (4S)-1-(3-hydroxypropyl)azepan-4-aminium trifluoroacetate (38 mg, 0.133 mmol) and triethylamine (0.034 mL, 0.244 mmol) in acetonitrile (1.0 mL) was added N,N′-disuccinimidyl carbonate (63 mg, 0.244 mmol) and the reaction mixture was stirred at room temperature overnight. The reaction mixture was concentrated under vacuum and the pale yellow solid residue was triturated with ether (3×) to afford the title compound which was used without purification in the next step.

### Step 2: (1S,5R)-2-({[(4S)-1-(3-Hydroxypropyl)azepan-4-yl]amino}carbonyl)-7-oxo-2,6-diazabicyclo-[3.2.0]heptane-6-sulfonic acid

A solution of (1S,5R)-2-7-oxo-2,6-diazabicyclo[3.2.0]heptane-6-sulfonic acid (47.7 mg, 0.248 mmol) and sodium bicarbonate (33.9 mg, 0.404 mmol) in water (2 mL) was added to a solution of the product of step 1 (theoretical amount of starting material present is 41.7 mg, 0.133 mmol) in acetonitrile (2 mL). The reaction mixture was stirred at room temperature overnight then concentrated under vacuum and purified by HPLC (21.2×250 mm Phenomenex Synergi Polar-RP 80A column; 10 micron; 35 mL/minute; 210 nM; 0% to 10% methanol/water over 9 minutes; title compound elutes at 10% methanol/water). The fractions containing product were collected and lyophilized overnight to afford off-white crystals (31.7 mg) which were triturated with acetonitrile (2×) to afford the title compound as a white solid (29.6 mg, 57%) which contained approximately 4% of an undetermined impurity which appears to be derived from triethylamine). ¹H NMR (500 MHz, D₂O) δ ppm 5.22 (1H, d, J=4 Hz), 4.74 (1H, dd, J=4 Hz), 3.92 (1H, dd, J=10, 9 Hz), 3.80-3.88 (1H, m), 3.67 (2H, t, J=6 Hz), 3.20-3.50 (7H, m), 2.39 (1H, dd, J=14, 6 Hz), 1.80-2.20 (8H, m), 1.58-1.68 (1H, m). LC-MS (neg. ionization) m/e 389 (M−H).

### Example 13

### (1S,5R)-2-({[(4S)-1-(2-Aminoethyl)azepan-4-yl]amino}carbonyl)-7-oxo-2,6-diazabicyclo-[3.2.0]heptane-6-sulfonic acid

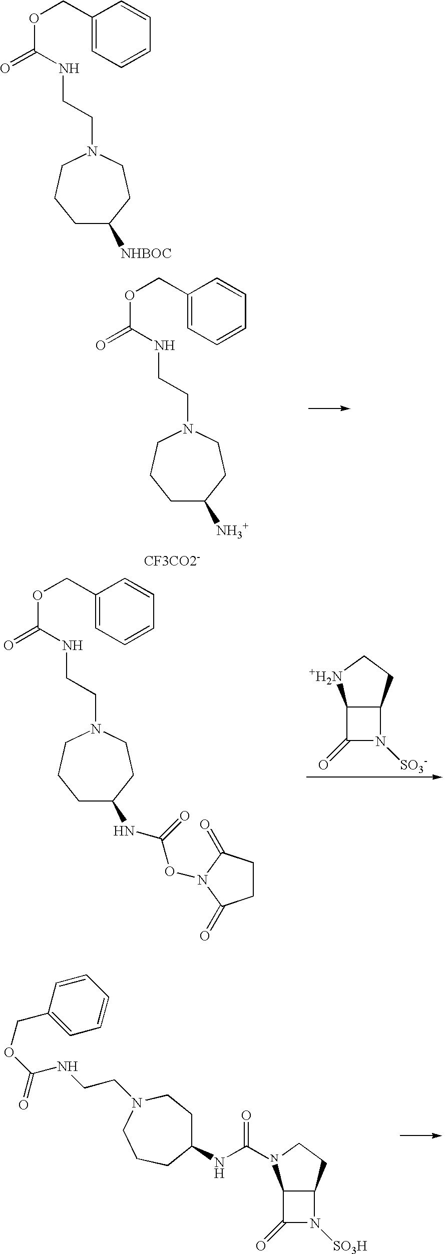

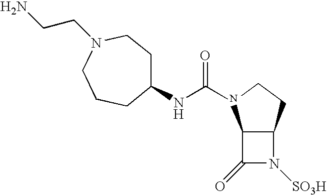

### Step 1: (4S)-1-(2-{[(Benzyloxy)carbonyl]amino}ethyl)azepan-4-aminium trifluoroacetate

Crude benzyl (2-{(4S)-4-[(tert-butoxycarbonyl)amino]azepan-1-yl}ethyl)carbamate (181 mg; theoretical amount present=157 mg=0.477 mmol) was dissolved in dichloromethane (2 mL) and trifluoroacetic acid (1 mL) was added. The reaction was stirred at room temperature overnight then concentrated under vacuum to afford the title compound (127 mg, 66%). The crude product was azeotroped with toluene and used without purification in the next step.

### Step 2: Benzyl {2-[(4S)-4-({[(2,5-dioxopyrrolidin-1-yl)oxy]carbonyl}amino)azepan-1-yl]ethyl}carbamate

To a solution of the product of step 1 (127 mg, 0.314 mmol) and triethylamine (0.077 mL, 0.554 mmol) in acetonitrile (3 mL) was added N,N′-disuccinimidyl carbonate (120 mg, 0.469 mmol) at room temperature. The resulting solution was stirred at room temperature overnight. The reaction was concentrated under vacuum and the residue was purified by Isco CombiFlash system: 12 g of MCI gel CHP20P (Supelco); 25 mL/minute flow rate; 210 nM wavelength; title compound elutes at 40% water/acetonitrile. Fractions containing the product were lyophilized over the weekend to afford the title compound (107.8 mg, 80%).

### Step 3: (1S,5R)-2-({[(4S)-1-(2-{[(benzyloxy)carbonyl]amino}ethyl)azepan-4-yl]amino}carbonyl)-7-oxo-2,6-diazabicyclo[3.2.0]heptane-6-sulfonic acid

A solution of (1S,5R)-7-oxo-2,6-diazabicyclo[3.2.0]heptane-6-sulfonic acid (54.3 mg, 0.283 mmol) and sodium bicarbonate (31 mg, 0.374 mmol) in water (1 mL) was added to a solution of the product of step 2 (107.8 mg, 0.249 mmol) in acetonitrile (1 mL). The reaction mixture was stirred at room temperature overnight. The reaction was concentrated under vacuum and the residue was purified by HPLC (30×100 mm Waters® Sunfire™ column; 5 micron; 35 mL/minute; 210 nM; 0% to 100% acetonitrile+0.05% TFA/water+0.05% TFA over 15 minutes; title compound elutes at 40-50% acetonitrile+0.05% TFA/water+0.05% TFA). Fractions containing the product were lyophilized overnight to afford the title compound as a white solid (62.5 mg, 49%).

### Step 4: (1S,5R)-2-({[(4S)-1-(2-Aminoethyl)azepan-4-yl]amino}carbonyl)-7-oxo-2,6-diazabicyclo-[3.2.0]heptane-6-sulfonic acid

The product of step 3 (41.8 mg, 0.082 mmol) was dissolved in methanol/water/acetic acid (5 mL/5 mL/5 drops). Palladium black (10.2 mg, 0.082 mmol) was then added and the reaction was subjected to 40 psi of hydrogen in a Parr Shaker overnight. The reaction was filtered through a microfilter to remove the catalyst. The filtrate was concentrated under vacuum and purified by HPLC (21.2×250 mm Phenomenex Synergi Polar-RP 80A column; 10 micron; 35 mL/minute; 210 nM; 0% to 15% methanol/water over 14 minutes; title compound eluted at 3% methanol/water). Fractions containing the product were lyophilized overnight to afford the title compound as a pale yellow sticky solid (21 mg, 68%). ¹H NMR (600 MHz, D₂O) δ ppm 5.21 (1H, d, J=4 Hz), (note: the anticipated signal at ˜4.75 ppm was obscured by large H₂O peak), 3.84-3.92 (2H, m), 3.30-3.50 (9H, m), 2.38 (1H, dd, J=14, 6 Hz), 1.85-2.20 (6H, m), 1.58-1.68 (1H, m). LC-MS (neg. ionization) m/e 374 (M−H).

### Example 14

### (1S,5R)-2-[({(4S)-1-[2-(Dimethylamino)ethyl]azepan-4-yl}amino)carbonyl]-7-oxo-2,6-diazabicyclo[3.2.0]heptane-6-sulfonic acid

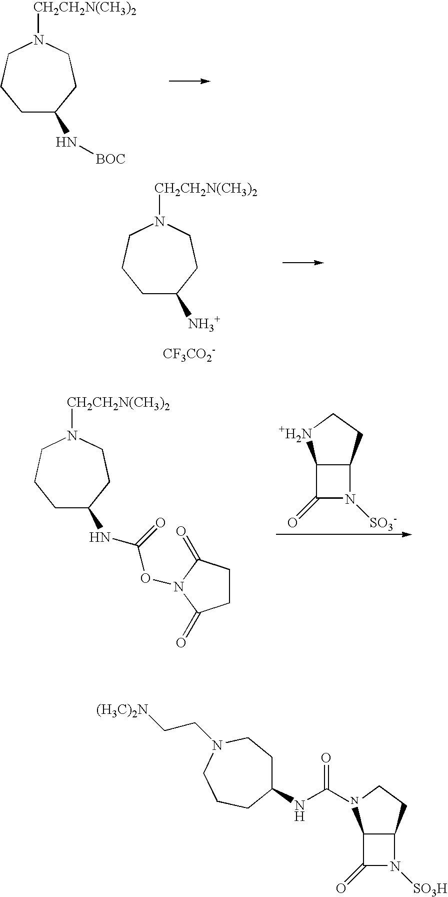

### Step 1: (4S)-1-[2-(dimethylamino)ethyl]azepan-4-aminium trifluoroacetate

Trifluoroacetic acid (1 mL) was added to a solution of tert-butyl {(4S)-1-[2-(dimethylamino)ethyl]-azepan-4-yl}carbamate (104 mg, 0.364 mmol) in dichloromethane (2 mL). The reaction was stirred at room temperature overnight then concentrated under vacuum to afford the title compound which was used without purification in the next step.

### Step 2: 1-{[({(4S)-1-[2-(Dimethylamino)ethyl]azepan-4-yl}amino)carbonyl]oxy}pyrrolidine-2,5-dione

To a solution of the product of step 1 (theoretical amount=62 mg, 0.364 mmol) in anhydrous acetonitrile (3 mL) was added Hunig's Base (0.13 mL, 0.744 mmol) followed by N,N′-disuccinimidyl carbonate (95.8 mg, 0.374 mmol) at room temperature. The resulting solution was stirred at room temperature over the weekend. The reaction was concentrated under vacuum and the residue was triturated with ether (3×). The resulting insoluble oil residue was dried under vacuum and used without further purification in the next step.

### Step 3: (1S,5R)-2-[({(4S)-1-[2-(Dimethylamino)ethyl]azepan-4-yl}amino)carbonyl]-7-oxo-2,6-diazabicyclo[3.2.0]heptane-6-sulfonic acid

A solution of (1S,5R)-7-oxo-2,6-diazabicyclo[3.2.0]heptane-6-sulfonic acid (77.6 mg, 0.404 mmol) and sodium bicarbonate (52.4 mg, 0.624 mmol) in water (1 mL) was added to a solution of the product of step 2 (theoretical amount=119 mg, 0.364 mmol) in acetonitrile (1 mL). The resulting mixture was stirred at room temperature overnight then concentrated under vacuum and purified by HPLC (21.2×250 mm Phenomenex Synergi Polar-RP 80A column; 10 micron; 35 mL/minute; 210 mM; 0% to 30% methanol/water over 14 minutes; title compound eluted at 20-25% methanol/water). Fractions containing product were lyophilized overnight to afford the title compound as a pale yellow solid which contained a small amount of an impurity. The solid was triturated with acetonitrile and the insoluble pale yellow solid was isolated by centrifugation to afford the title compound (3.5 mg, 2.4%). Additional title compound was obtained by concentration of the supernatant from the wash of the lyophilized HPLC fractions. The resulting white solid was triturated with ether and collected by centrifugation to afford additional title compound (11.4 mg, 7.8%) which was less pure than the first batch. ¹H NMR (500 MHz, D₂O) δ ppm 5.22 (1H, d, J=4 Hz), 4.73 (1H, dd, J=4 Hz), 3.91 (1H, dd, J=10, 9 Hz), 3.79-3.84 (1H, m), 3.28-3.35 (1H, m), 2.95-3.18 (8H, m), 2.64 (6H, m), 2.39 (1H, dd, J=14, 6 Hz), 1.55-2.10 (7H, m). LC-MS (neg. ionization) m/e 402 (M−H).

### Example 15

### (1S,5R)-7-oxo-2-{[(2,2,7,7-Tetramethylazepan-4-yl)amino]carbonyl}-2,6-diazabicyclo-[3.2.0]-heptane-6-sulfonic acid

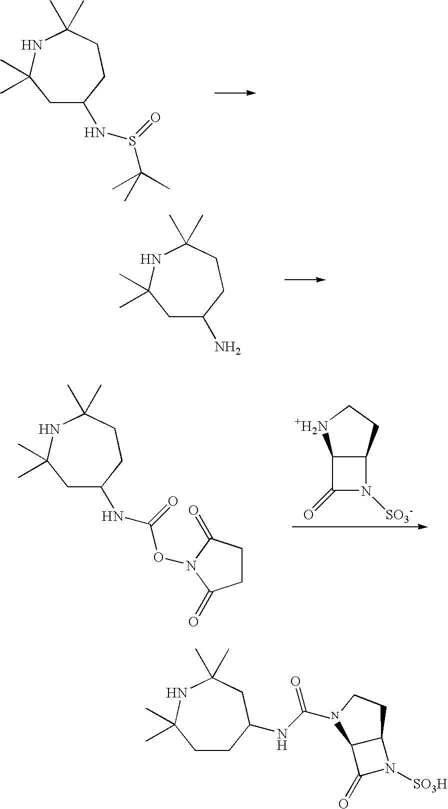

### Step 1: 2,2,7,7-Tetramethylazepan-4-amine:

To a solution of 2-methyl-N-(2,2,7,7-tetramethylazepan-4-yl)propane-2-sulfinamide (83.4 mg, 0.304 mmol) in methanol (2 mL) was added 4N hydrogen chloride in dioxane (0.1 mL, 0.400 mmol) at room temperature. After 1 hour, an additional 0.3 mL of 4N hydrogen chloride in dioxane was added. After another hour the reaction mixture was concentrated under vacuum and the residue was triturated with ether. The insoluble oil was azeotroped with toluene and used without purification in the next step.

### Step 2: 1-({[(2,2,7,7-Tetramethylazepan-4-yl)amino]carbonyl}oxy)pyrrolidine-2,5-dione

To a solution of the product of step 1 (theoretical amount=52 mg, 0.304 mmol) in anhydrous acetonitrile (3 mL) was added Hunig's Base (0.12 mL, 0.6874 mmol) followed by N,N′-disuccinimidyl carbonate (79.8 mg, 0.312 mmol) at room temperature. The resulting solution was stirred at room temperature overnight. The reaction was concentrated under vacuum and the residue was triturated with ether (3×). The resulting insoluble oil residue was dried under vacuum and used without further purification in the next step.

### Step 3: (1S,5R)-7-oxo-2-{[(2,2,7,7-Tetramethylazepan-4-yl)arnino]carbonyl}-2,6-dione diazabicyclo-[3.2.0]-heptane-6-sulfonic acid

A solution of (1S,5R)-7-oxo-2,6-diazabicyclo[3.2.0]heptane-6-sulfonic acid (64.3 mg, 0.334 mmol) and sodium bicarbonate (38.3 mg, 0.456 mmol) in water (1 mL) was added to a solution of the product of step 2 (theoretical amount=95 mg, 0.304 mmol) in acetonitrile (1 mL). The reaction was stirred at room temperature overnight then concentrated under vacuum and purified by HPLC (21.2×250 mm Phenomenex Synergi Polar-RP 80A column; 10 micron; 35 mL/minute; 210 nM; 0% to 30% methanol/water over 14 minutes; title compound eluted at 20-25 % methanol/water). Fractions containing product were combined and lyophilized overnight to afford the title compound as a white solid (10.3 mg, 8.7%). ¹H NMR (500 MHz, D₂O) δ ppm 5.22 (1H, d, J=4 Hz), 4.74 (1H, dd, J=4 Hz), 3.86-3.93 (2H, m), 3.29-3.36 (1H, m), 2.40 (1H, dd, J=14, 6 Hz), 1.87-2.07 (6H, m), 1.70-1.80 (1H, m), 1.54 (3H, s), 1.45 (3H, s), 1.43 (6H, s). LC-MS (neg. ionization) m/e 387 (M−H).

### Example 16

### (1S,5R)-2-[(Azocan-5-ylamino)carbonyl]-7-oxo-2,6-diazabicyclo-[3.2.0]-heptane-6-sulfonic acid

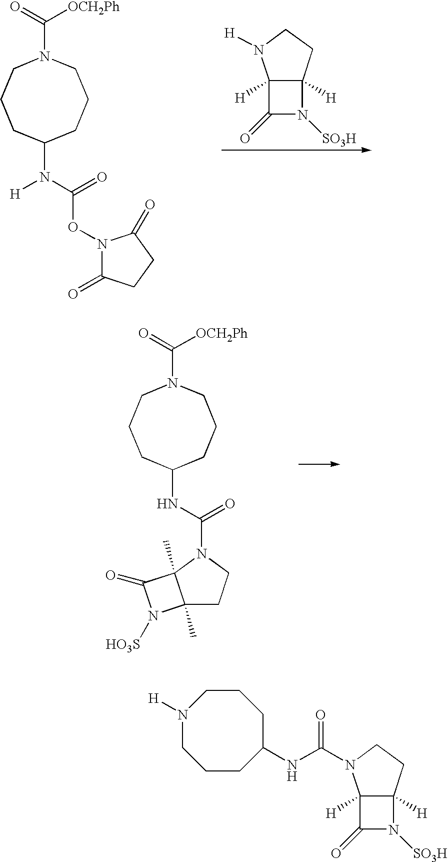

### Step 1: (1S,5R)-2-[({-1-[(Benzyloxy)carbonyl]azocan-4-yl}amino)carbonyl]-7-oxo-2,6-diazabicyclo[3.2.0]heptane-6-sulfonic acid

Using (1S,5R)-7-oxo-2,6-diazabicyclo[3.2.0]heptane-6-sulfonic acid (0.172 g, 0.89 mmol) and benzyl 4-({[(2,5-dioxopyrrolidin-1-yl)oxy]-carbonyl}amino)-azocane-1-carboxylate (0.36 g, 0.89 mmol) in the procedure outlined in Step 1 of Example 1, the title compound was obtained as a white solid (0.15 g, 35%) after lyophilization.

### Step 2: (1S,5R)-2-[(Azocan-5-ylamino)carbonyl]-7-oxo-2,6-diazabicyclo-[3.2.0]-heptane-6-sulfonic acid

Applying the procedure of Step 2 of Example 1 to (1S,5R)-2-[({-1-[(benzyloxy)carbonyl]azocan-4-yl}amino)carbonyl]-7-oxo-2,6-diazabicyclo[3.2.0]heptane-6-sulfonic acid (0.050 g, 0.10 mmol) afforded the title compound as a white solid (0.014 g, 40%) after lyophilization. ¹H NMR (500 MHz, D₂O) δ ppm 5.23 (1H, d, J=4 Hz), (note: the anticipated signal at ˜4.75 ppm was obscured by large H₂O peak), 3.91 (1H, dd, J=10, 10 Hz), 3.75-3.85 (1H, m), 3.25-3.38 (3H, m), 3.15-3.24 (2H, m), 2.39 (1H, dd, J=14, 6 Hz), 1.80-2.18 (7H, m), 1.68-1.78 (2H, m).

### Example 17

### (1S,5R)-2-[(Azocan-4-ylamino)carbonyl]-7-oxo-2,6-diazabicyclo[3.2.0]heptane-6-sulfonic acid (Isomer A)

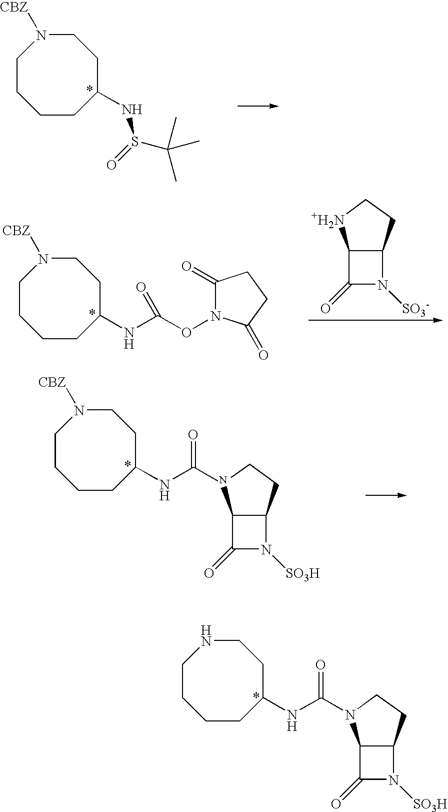

### Step 1: Benzyl 4-({[(2,5-dioxopyrrolidin-1-yl)oxy]carbonyl}amino)azocane-1-carboxylate (Isomer A)

To a solution of tert-butyl 4-{[(R)-tert-butylsulfinyl]amino}azocane-1-carboxylate (20 mg, 0.0.055 mmol) in methanol was added 4N hydrogen chloride in dioxane (0.014 mL, 0.056 mmol) at room temperature. After 20 minutes the reaction mixture was concentrated under vacuum and the residue was triturated with ether. The insoluble oil was dissolved in acetonitrile then triethyl amine (0.009 mL, 0.066 mmol) and N,N′-disuccinimidyl carbonate (17 mg, 0.066 mmol) were added. The resulting solution was stirred at room temperature for 2 hours then concentrated under vacuum. The residue was used without purification in the next step.

### Step 2: (1S,5R)-2-[({1-[(Benzyloxy)carbonyl]azocan-4-yl}amino)carbonyl]-7-oxo-2,6-diazabicyclo[3.2.0]heptane-6-sulfonic acid (Isomer A)

A solution of (1S,5R)-7-oxo-2,6-diazabicyclo[3.2.0]heptane-6-sulfonic acid (37 mg, 0.194 mmol) and sodium bicarbonate (5 mg, 0.060 mmol) in water (1 mL) was added to a solution of the product of step 1 (theoretical amount=26 mg, 0.055 mmol) in acetonitrile (3 mL). The reaction was stirred at room temperature for 4 hours then concentrated under vacuum and purified by HPLC (Sunfire column). Fractions containing product were combined and lyophilized overnight to afford the title compound as a white solid (10 mg, 39%).

### Step 3: (1S,5R)-2-[(Azocan-4-ylamino)carbonyl]-7-oxo-2,6-diazabicyclo[3.2.0]heptane-6-sulfonic acid (Isomer A)

The product of step 2 (10 mg, 0.021 mmol) ) was dissolved in ethanol (5 mL)/water (0.5 mL)/acetic acid (1 drop) then 20% palladium hydroxide on carbon was added and the reaction was subjected to 40 psi of hydrogen in a Parr Shaker for 4 hours. The reaction was filtered to remove the catalyst and the filtrate was concentrated under vacuum and purified by HPLC (21.2×250 mm Phenomenex Synergi Polar-RP 80A column). Fractions containing the product were lyophilized overnight to afford the title compound (4.5 mg, 63%). ¹H NMR (500 MHz, D₂O) δ ppm 5.22 (1H, d, J=4 Hz), 4.74 (1H, dd, J=4 Hz), 3.91 (1H, dd, J=10, 9 Hz), 3.82-3.87 (1H, m), 3.21-3.41 (3H, m), 3.15-3.19 (2H, m), 2.39 (1H, dd, J=14, 6 Hz), 2.09-2.14 (1H, m), 1.80-2.22 (6H, m), 1.60-1.68 (2H, m).

### Example 18

### (1S,5R)-2-[(Azocan-4-ylamino)carbonyl]-7-oxo-2,6-diazabicyclo[3.2.0]heptane-6-sulfonic acid (Isomer B)

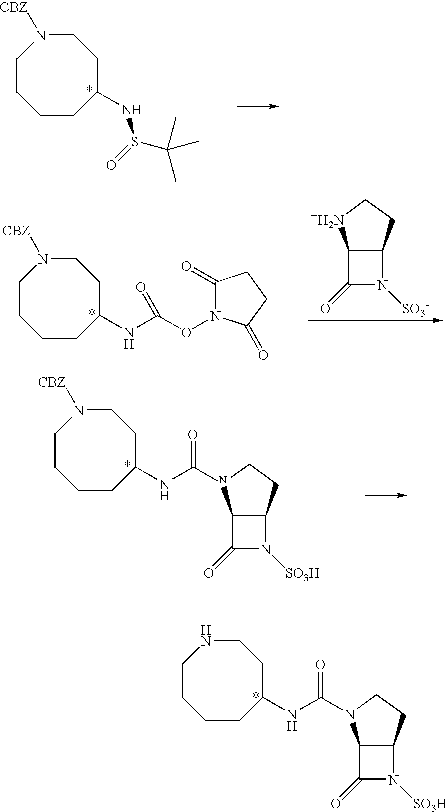

### Step 1: Benzyl 4-({[(2,5-dioxopyrrolidin-1-yl)oxy]carbonyl}amino)azocane-1-carboxylate (Isomer B)

To a solution of tert-butyl 4-{[(R)-tert-butylsulfinyl]amino}azocane-1-carboxylate (48 mg, 0.131 mmol) in methanol was added 4N hydrogen chloride in dioxane (0.033 mL, 0.132 mmol) at room temperature. After 20 minutes the reaction mixture was concentrated under vacuum and the residue was triturated with ether. The insoluble oil was dissolved in acetonitrile then triethyl amine (0.032 mL, 0.23 mmol) and N,N′-disuccinimidyl carbonate (60 mg, 0.23 mmol) were added. The resulting solution was stirred at room temperature for 2 hours then concentrated under vacuum. The residue was used without purification in the next step.

### Step 2: (1S,5R)-2-[({1-[(Benzyloxy)carbonyl]azocan-4-yl}amino)carbonyl]-7-oxo-2,6-diazabicyclo-[3.2.0]heptane-6-sulfonic acid (Isomer B)

A solution of (1S,5R)-7-oxo-2,6-diazabicyclo[3.2.0]heptane-6-sulfonic acid (37 mg, 0.194 mmol) and sodium bicarbonate (16 mg, 0.194 mmol) in water (1 mL) was added to a solution of the product of step 1 (theoretical amount=53 mg, 0.131 mmol) in acetonitrile (4 mL). The reaction was stirred at room temperature overnight then concentrated under vacuum and purified by HPLC (Sunfire column). Fractions containing product were combined and lyophilized overnight to afford the title compound as a white solid (7 mg, 11%).

### Step 3: (1S,5R)-2-[(Azocan-4-ylamino)carbonyl]-7-oxo-2,6-diazabicyclo[3.2.0]heptane-6-sulfonic acid (Isomer B)

The product of step 2 (7 mg, 0.015 mmol) ) was dissolved in ethanol (5 mL)/water (0.5 mL)/acetic acid (1 drop) then 20% palladium hydroxide on carbon was added and the reaction was subjected to 40 psi of hydrogen in a Parr Shaker overnight. The reaction was filtered to remove the catalyst and the filtrate was concentrated under vacuum and purified by HPLC (21.2×250 mm Phenomenex Synergi Polar-RP 80A column). Fractions containing the product were lyophilized overnight to afford the title compound (5 mg, 100%). ¹H NMR (500 MHz, D₂O) δ ppm 5.23 (1H, d, J=4 Hz), 4.74 (1H, dd, J=4 Hz), 3.91 (1H, dd, J=10, 9 Hz), 3.82-3.87 (1H, m), 3.21-3.41 (3H, m), 3.15-3.19 (2H, m), 2.39 (1H, dd, J=14, 6 Hz), 2.09-2.14 (1H, m), 1.80-2.22 (6H, m), 1.60-1.68 (2H, m).

### Example 19

### (1S,5R)-2-{[Azonan-5-ylamino]carbonyl}-7-oxo-2,6-diazabicyclo[3.2.0]heptane-6-sulfonic acid (Isomer A)

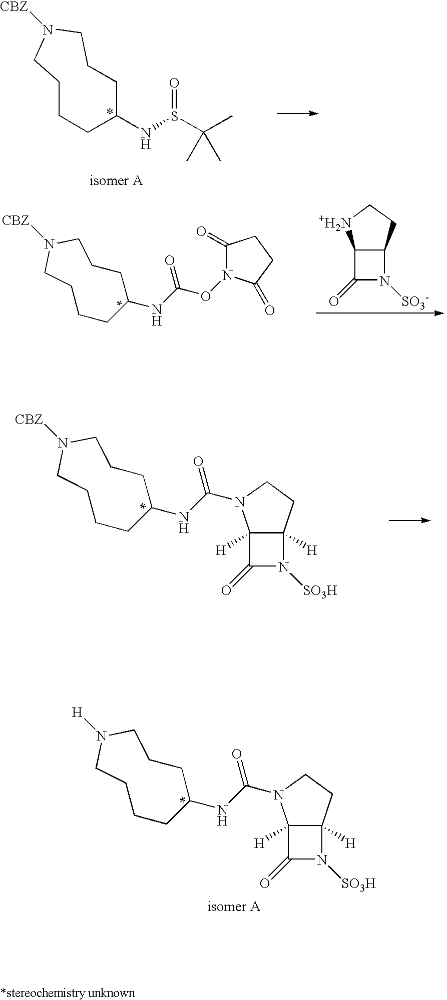

### Step 1: Benzyl 5-({[(2,5-dioxopyrrolidin-1-yl)oxy]carbonyl}amino)azonane-1-carboxylate (Isomer A)

To a solution of tert-butyl 5-{[(R)-tert-butylsulfinyl]amino}azonane-1-carboxylate (32 mg, 0.084 mmol, isomer A) in methanol was added 4N hydrogen chloride in dioxane (0.021 mL, 0.084 mmol) at room temperature. After 15 minutes the reaction mixture was concentrated under vacuum and the residue was triturated with ether. The insoluble oil was dissolved in acetonitrile then triethylamine (0.014 mL, 0.10 mmol) and N,N′-disuccinimidyl carbonate (26 mg, 0.10 mmol) were added. The resulting solution was stirred at room temperature for 2 hours then concentrated under vacuum. The residue was used without purification in the next step.

### Step 2: (1S,5R)-2-[({1-[(Benzyloxy)carbonyl]azonan-5-yl}amino)carbonyl]-7-oxo-2,6-diazabicyclo[3.2.0]heptane-6-sulfonic acid (Isomer A)

A solution of (1S,5R)-7-oxo-2,6-diazabicyclo[3.2.0]heptane-6-sulfonic acid (16 mg, 0.084 mmol) and sodium bicarbonate (7 mg, 0.084 mmol) in water (1 mL) was added to a solution of the product of step 1 (theoretical amount=35 mg, 0.084 mmol) in acetonitrile (4 mL). The reaction was stirred at room temperature overnight then concentrated under vacuum and purified by HPLC (Sunfire column). Fractions containing product were combined and lyophilized overnight to afford the title compound as a white solid (30 mg, 72%).

### Step 3: (1S,5R)-2-{[Azonan-5-ylamino]carbonyl}-7-oxo-2,6-diazabicyclo[3.2.0heptane-6-sulfonic acid (Isomer A)

The product of step 2 (30 mg, 0.061 mmol) ) was dissolved in ethanol (3 mL)/water (2 drops)/acetic acid (1 drop) then 20% palladium hydroxide on carbon (20 mg) was added and the reaction was subjected to 45 psi of hydrogen in a Parr Shaker overnight. The reaction was filtered to remove the catalyst and the filtrate was concentrated under vacuum and purified by HPLC (21.2×250 mm Phenomenex Synergi Polar-RP 80A column). Fractions containing the product were lyophilized overnight to afford the title compound (3 mg, 14%). ¹H NMR (500 MHz, D₂O) δ ppm 5.23 (1H, d, J=4 Hz), 4.74 (1H, dd, J=4 Hz), 3.91 (1H, dd, J=10, 9 Hz), 3.81-3.86 (1H, m), 3.29-3.35 (1H, m), 3.19-3.26 (4H, m), 2.39 (1H, dd, J=14, 6 Hz), 1.61-2.06 (11H, m). LC-MS (neg. ionization) m/e 359 (M−H).

### Example 20

### (1S,5R)-2-{[Azonan-5-ylamino]carbonyl}-7-oxo-2,6-diazabicyclo[3.2.0]heptane-6-sulfonic acid (Isomer B)

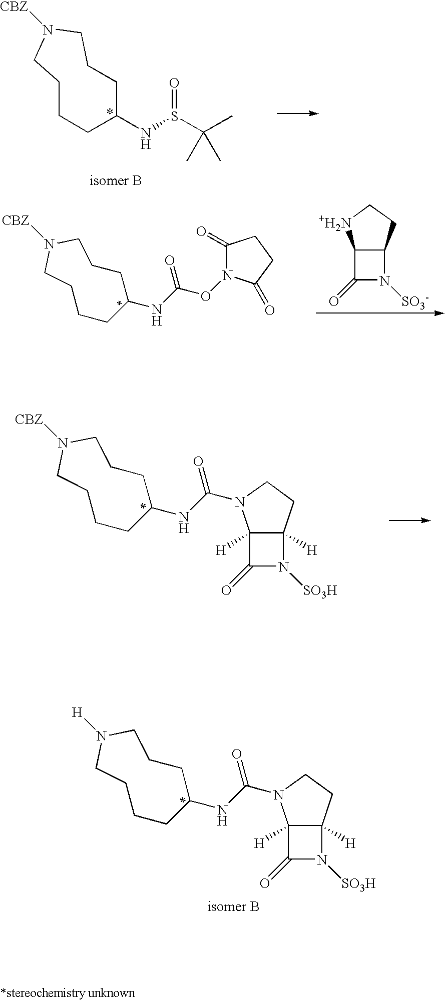

### Step 1: Benzyl 5-({[(2,5-dioxopyrrolidin-1-yl)oxy]carbonyl}amino)azonane-1-carboxylate (Isomer B)

To a solution of tert-butyl 5-{[(R)-tert-butylsulfinyl]amino}azonane-1-carboxylate (45 mg, 0.12 mmol, isomer B) in methanol was added 4N hydrogen chloride in dioxane (0.030 mL, 0.12 mmol) at room temperature. After 15 minutes the reaction mixture was concentrated under vacuum and the residue was triturated with ether. The insoluble oil was dissolved in acetonitrile then triethylamine (0.020 mL, 0.14 mmol) and N,N′-disuccinimidyl carbonate (36 mg, 0.14 mmol) were added. The resulting solution was stirred at room temperature for 2 hours then concentrated under vacuum. The residue was purified by HPLC (Sunfire column) to afford the title compound as an oil (10 mg, 20%).

### Step 2: (1S,5R)-2-[({1-[(Benzyloxy)carbonyl]azonan-5-yl }amino)carbonyl]-7-oxo-2,6-diazabicyclo[3.2.0]heptane-6-sulfonic acid (Isomer B)

A solution of (1S,5R)-7-oxo-2,6-diazabicyclo[3.2.0]heptane-6-sulfonic acid (4.6 mg, 0.024 mmol) and sodium bicarbonate (7 mg, 0.084 mmol) in water (1 mL) was added to a solution of the product of step 1 (10 mg, 0.024 mmol) in acetonitrile (4 mL). The reaction was stirred at room temperature overnight then concentrated under vacuum and purified by HPLC (Sunfire column). Fractions containing product were combined and lyophilized overnight to afford the title compound as a white solid (5 mg, 43%).

### Step 3: (1S,5R)-2-{[Azonan-5-ylamino]carbonyl}-7-oxo-2,6-diazabicyclo[3.2.0]heptane-6-sulfonic acid (Isomer B)

The product of step 2 (5 mg, 0.010 mmol) ) was dissolved in ethanol (2 mL)/water (1 drops)/acetic acid (1 drop) then 20% palladium hydroxide on carbon (5 mg) was added and the reaction was subjected to 45 psi of hydrogen in a Parr Shaker for 4 hours. The reaction was filtered to remove the catalyst and the filtrate was concentrated under vacuum and purified by HPLC (21.2×250 mm Phenomenex Synergi Polar-RP 80A column). Fractions containing the product were lyophilized overnight to afford the title compound (2 mg, 55%). ¹H NMR (500 MHz, D₂O) δ ppm 5.23 (1H, d, J=4 Hz), 4.74 (1H, dd, J=4 Hz), 3.91 (1H, dd, J=10, 9 Hz), 3.81-3.86 (1H, m), 3.29-3.35 (1H, m), 3.19-3.26 (4H, m), 2.39 (1H, dd, J=14, 6 Hz), 1.61-2.06 (11H, m). LC-MS (neg. ionization) m/e 359 (M−H).

### Examples 21-31

Applying the procedure of Example 1 to the starting materials in the following Table, the following compounds can be prepared (note: a protecting group is not necessary for the compounds of Examples 22-25 and thus the deprotection step set forth in Example 1 can be omitted for them):

| Example | Activated Side Chain | Product |
|---|---|---|
| 21 | 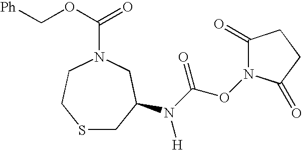 | 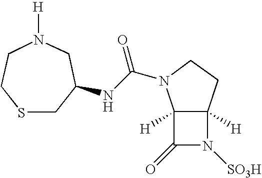 |
| 22 | 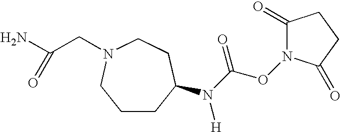 | 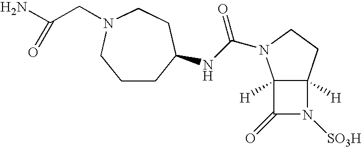 |
| 23 | 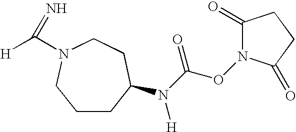 | 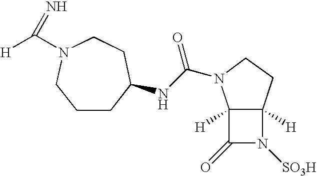 |
| 24 | 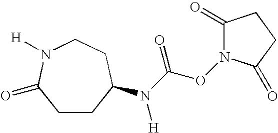 | 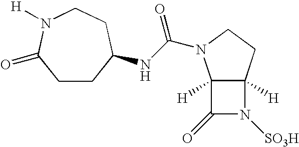 |
| 25 | 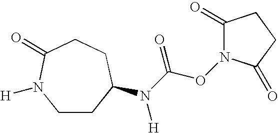 | 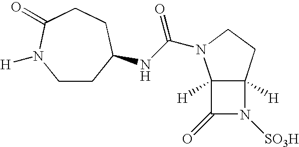 |
| 26 | 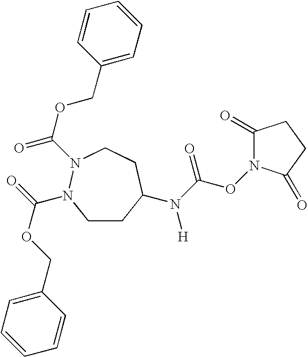 | 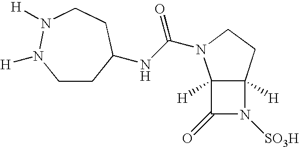 |
| 27 | 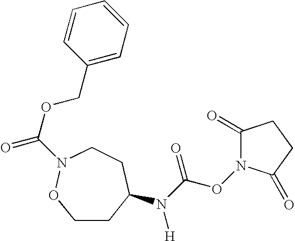 | 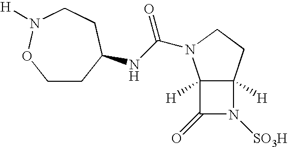 |
| 28 | 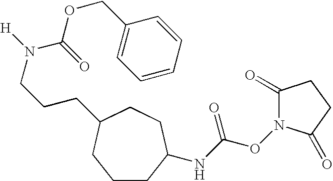 | 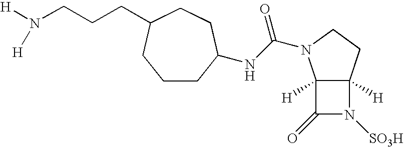 |
| 29 | 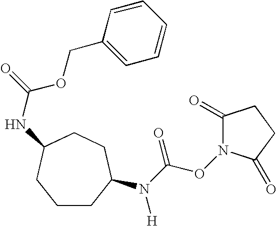 | 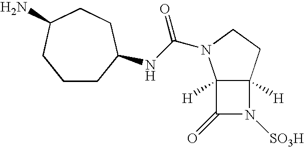 |
| 30 | 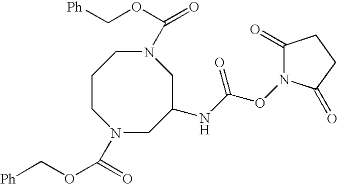 | 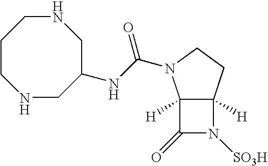 |
| 31 | 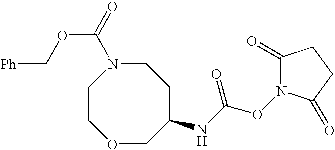 | 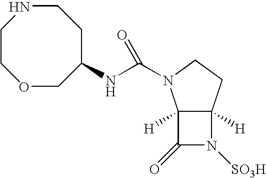 |

### Example 32

### Enzyme Activity: Determination of IC₅₀

The Class C enzyme activities were measured in the presence of the test inhibitor in spectrophotometric assay against the commercially available substrate, nitrocefin. The enzyme AmpC (P. aeruginosa.), and the substrate, were dissolved in 100 mM KH₂PO₄ buffer (pH 7). The buffer also contains 0.005% BSA. The test inhibitor was dissolved in DMSO and diluted 1:20 in the assay, resulting in a final concentration range of 50 μM to 0.0002 μM. In a 96-well microplate, the test inhibitor was incubated with the beta-lactamase enzyme for 40 minutes at ambient temperature, the substrate solution was added, and the incubation continued for another 40 minutes. The spectrophotomertric reaction was quenched by the addition of 2.5N acetic acid and the absorbance at 492 nm was measured. The IC₅₀ value was determined from semi logarithmic plots of enzyme inhibition versus inhibitor concentration, with a curve generated using a 4-parameter fit.

Representative compounds of the present invention exhibit inhibition of Class C β-lactamase in this assay. For example, the compounds of Examples 1 to 20 were tested in this assay and were found to have IC₅₀ values in a range of about 25 micromolar or less. The IC₅₀ values of selected compounds are shown in Table 1.

### Synergy Assay Protocol:

The assay determines the concentration of a β-lactamase inhibitor required to reduce the MIC of a β-lactam antibiotic by one-half, one-quarter, one-eighth, one-sixteenth and one-thirty-second against strains of bacteria normally resistant to the antibiotic in question. This is accomplished by titrating the BLI in a serial dilution across a microtiter plate while at the same time titrating the antibiotic in a serial dilution down the microtiter plate and then inoculating the plate with the bacterial strain in question and allowing the bacteria to grow up overnight. Each well in this microplate checkerboard contains a different combination of concentrations of the inhibitor and the antibiotic allowing a full determination of any synergy between the two.

### Bacterial Strain/Antibiotic Combinations:

CL 5701 (Pseudomonas aeruginosa; Pa AmpC)/Imipenem
MB 2646 (Enterobacter cloacae; P99)/Ceftazidime
CL 5513 (Klebsiella pneumoniae; SHV-5)/Ceftazidime
CL 6188 (Acinetobacter baumanii; Oxa40)/Imipenem
CL 6569 (Klebsiella pneumoniae; KPC-2)/Imipenem
CL 5761 (Klebsiella pneumoniae; KPC-3)/Imipenem
CLB 21648 (Acinetobacter baumanii; Ab AmpC)/Imipenem

### General Checkerboard Method:

1. All wells in rows B-H of MIC 2000 microtiter plates are filled with 100 μL of MHBII+1% DMSO (dimethyl sulfoxide).
2. All wells in row A of MIC 2000 microtiter plates are filled with 100 μL of 2× MHBII+2% DMSO.
3. 100 μL of 4× the final antibiotic concentration wanted is added to well A1 of the MIC 2000 plates.
4. 100 μL of 2× the final antibiotic concentration wanted is added to wells A2-A12 of the MIC 2000 plates.
5. 100 μL is serially diluted from row A to row G of each MIC 2000 plate.
6. 100 μL is removed from each well in row G of each MIC 2000 plate.
7. 100 μL of 2× the final inhibitor concentration wanted (in MHBII+1% DMSO) is added to all wells in column 1 of the microtiter plates.
8. 100 μL is serially diluted from column 1 to column 11 of each MIC 2000 plate.
9. 100 μL is removed from each well in column 11 of each MIC 2000 plate.
10. Plates are then inoculated with an overnight growth (in TSB) of the strain to be tested using an MIC 2000 inoculator.
11. Plates are left at 37° C. for about 20 hours and scored for growth by eye.

### Media (All are Sterilized by Autoclaving Prior to Any Addition of DMSO):

### MHBII+1% DMSO

| | | |
|---|---|---|
| Mueller Hinton Broth type II cation adjusted | 4.4 | g |
| (BBL ™) | | |
| DMSO | 2.0 | mL |
| Distilled water | 198.0 | mL |

### 2× MHBII+2% DMSO

| | | |
|---|---|---|
| Mueller Hinton Broth type II cation adjusted | 8.8 | g |
| (BBL ™) | | |
| DMSO | 4.0 | mL |
| Distilled water | 196.0 | mL |

### 1.02× MHBII

| | | |
|---|---|---|
| Mueller Hinton Broth type II cation adjusted | 4.4 | g |
| (BBL ™) | | |
| Distilled water | 198.0 | mL |

### 1.1× MHBII +1% DMSO

| | | |
|---|---|---|
| Mueller Hinton Broth type II cation adjusted | 4.4 | g |
| (BBL ™) | | |
| DMSO | 2.0 | mL |
| Distilled water | 178.0 | mL |

### TSB

Trypticase Soy Broth (BBL™) Prepared as Directed on Bottle.

Synergy may be expressed as a ratio of the minimum inhibitory concentration (MIC) of an antibiotic tested in the absence of a β-lactamase inhibitor to the MIC of the same antibiotic tested in the presence of the β-lactamase inhibitor. A ratio of one (1) indicates that the β-lactamase inhibitor has no effect on antibiotic potency. A ratio greater than one (1) indicates that the β-lactamase inhibitor produces a synergistic effect when co-administered with the antibiotic agent. The preferred β-lactamase inhibitors of the present invention exhibit a synergy ratio of at least about 2, more preferred compounds exhibit a ratio of at least about 4, still more preferably at least about 8, and most preferred at least about 16. Alternatively, the synergy effect may be expressed as a factor, again, utilizing a concentration of the BLI to lower the MIC of the antibiotic. Thus, if the MIC of the antibiotic is 20 μg/mL and a 1.5 μM concentration of BLI lowers the MIC to 5 μg/mL, the synergy effect is four fold or “4× synergy” at 1.5 μM of BLI.

Representative compounds of the present invention display a synergy effect. For example, the compounds of Examples 1 to 20 were determined to have 2× synergy concentrations in a range of from about 100 μM or less. The synergy concentrations of selected compounds of the invention against P. aeruginosa strain CL5701 are shown in Table 1.

TABLE 1
Biological Data

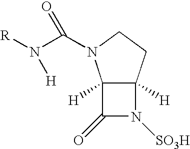

| | | | | | In Vitro |
|---|---|---|---|---|---|
| | | | In Vitro 2X | In Vitro 8X | 16X |
| | | P. aeruginosa | Synergy | Synergy | Synergy |
| | | AmpC IC₅₀ | CL5701 | CL5701 | CL5701 |
| Example | R | (μM) | (μM)² | (μM)² | (μM)² |
| 1 | 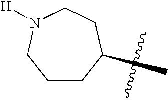 | 1.2 | 2.3 | 12.5 | 25 |
| 2 | 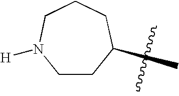 | 4 | 3.1 | 25 | 100 |
| 3 | 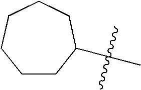 | 0.4 | 100 | >100 | >100 |
| 7 | 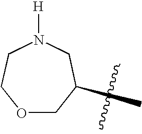 | 0.6 | 6.3 | 25 | 100 |
| 8 | 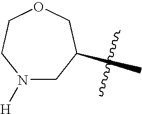 | 1.1 | 6.3 | 50 | 100 |
| 17 | 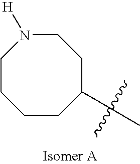 | 1.3 | 3.1 | 12.5 | 50 |
| 18 | 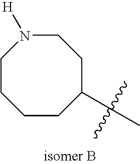 | 25 | 12.5 | >100 | >100 |
| Compound A¹ | 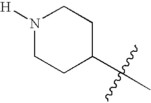 | 6.8 | 3.7 | 21 | 67 |

¹Compound A is from J. Med. Chem. 1997, 40: 335.
²These are the concentrations for 2X, 8X and 16X synergy with imipenem against P. aeruginosa strain CL5701. For example, a 12.5 μM concentration of the compound of Example 1 reduces the MIC of imipenem versus P. aeruginosa strain CL5701 by a factor of 8 (8X synergy).

X-ray crystal structures were obtained for the covalent enzyme/inhibitor complexes of (1S,5R)-2-{[(4S)-azepan-4-ylamino]carbonyl}-7-oxo-2,6-diazabicyclo[3.2.0]heptane-6-sulfonic acid (compound 1), (1S,5R)-2-{[(4R)-azepan-4-ylamino]carbonyl}-7-oxo-2,6-diazabicyclo[3.2.0]heptane-6-sulfonic acid (compound 2), and (1S,5R)-2-[(cyclohexylamino)carbonyl]-7-oxo-2,6-diazabicyclo[3.2.0]heptane-6-sulfonic acid (comparison compound from the literature) bound to AmpC, a class C beta-lactamase. The X-ray structures indicate that an improved interaction of the side chain nitrogen of compound 1 with an amino acid of the enzyme is the structural basis for the superior beta-lactamase activity of compound 1.

### Example 33

### (1S,5R)-2-{[(4S)-azepan-4-ylamino]carbonyl}-7-oxo-2,6-diazabicyclo-[3.2.0-heptane-6-sulfonic acid

### Step 1: CBZ-Protected Hydroxy Proline

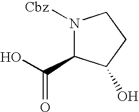

A 75 L round bottom flask equipped with a banana stirblade was charged with hydroxy proline (4.7 kg; 35.8 moles; 1.0 eq.) (free flowing solids) and then with water (18.8 L). The internal temperature of the flask after charging was 13° C. Room-temperature NaOH (10M, 9.5 kg/7.17 L; 2.0 eqs.) was then charged to the flask to provide a homogeneous, clear, pale yellow solution having a temperature of 20° C. CBZ-Cl (6.4 kg/5.37 L; 37.6 moles; 1.05 eqs.) was then slowly charged to the flask (turning the solution cloudy) over 1.75 hours while maintaining the internal temperature at about 25° C. using a 0° C. salt/ice bath. The solution was then aged for 30 minutes, after which a solution sample was assayed by LC, wherein the desired product was present at 94.8% (area percent), benzyl alcohol at 2.6%, and an impurity at 2.2%. The reaction was slowly quenched by the addition of concentrated HCl (1.5 L) over 35 minutes, while maintaining the internal temperature at 22° C. (no exotherm was observed), to provide a solution with pH=3.6. The solution was then seeded with about 10 g of product from a previous batch (Note—the procedure does not require seed), and the resulting slurry was aged for 1.25 hours, during which time the slurry thickened substantially. More HCl (1.88 L) was added over 50 minutes and the solution was aged for an additional 25 minutes. The slurry was filtered under vacuum, the wetcake washed with water (2×18 L), and the solids dried at room temperature in a filter pot under nitrogen while applying a vaccum thereto for several days. The title product was isolated in 96.5% yield (9.17 kg, 98.2 A %).

HPLC Assay Conditions: column=Zorbax RX-C8, 4.6×250 mm; temperature=20.1 ° C.; flow rate=1.0 mL/minute; detector=210, 254 nm; mobile phase=Solvent A: acetonitrile, Solvent B: 0.1% H₃PO₄; gradient=

| Time | | |
|---|---|---|
| (min.) | A % | B % |
| 0 | 10 | 90 |
| 4 | 45 | 55 |
| 6 | 45 | 55 |
| 10 | 0 | 100 |
| 11.5 | 0 | 100 |

### Step 2: CBZ-Proline Amide

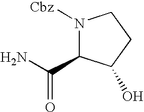

THF (13.5 L) was charged to a 100 L round bottom flask equipped with a banana stirblade and containing a mixture of NH₄HCO₃ (2.01 kg; 25.4 moles; 1.5 eqs) and a first portion of CBZ-protected hydroxy proline (0.45 kg; 1.69 moles; 0.1 eq.) at room temperature. Pyridine (0.56 kg; 7.1 moles; 0.4 eq.; d=0.978) was then added to the cloudy solution, which was then heated to 50° C. and allowed to age for 1 hour, during which time the solution thickened to a milky white slurry. A solution of Boc₂O (4.81 kg; 22 moles; 1.3 eqs.) in THF (4.5 L) and a second portion of Cbz acid (4.05 kg; 15.21 moles; 0.9 eq.) were added simultaneously over 1 hour. The reaction was monitored by LC after completion of the addition and, when the reaction was determined to be complete—about 15 minutes after completion of the addition—, the solution was cooled to room temperature with an ice bath and then filtered through a 20, 10, and 5 μm pore size in-line filter. The clear, light yellow solution was allowed to stand overnight.

The following day, the solution was transferred to the round bottom flask via vacuum and then heated to 50° C., after which heptanes (7 L) were charged to the hot solution and then the solution was seeded with Cbz-protected amide (315 g) from a previous batch (Note: the procedure does not require seed). Additional heptanes (6.3 L) were added to the thin slurry which was then allowed to age at 50° C. for 100 minutes. The thickened slurry was then cooled to room temperature over the course of 2.5 hours using a water bath, after which the content of the desired CBZ-protected amide in the mother liquor was determined by LC to be 45.9 mg/g. Two additional portions of heptanes (32 L and 5.7 L) were added, each followed by an LC assay of the mother liquor, and the final mother liquor concentration was determined to be 3.1 mg/g. The slurry was then transferred to a 10-inch filter pot via suction, the resulting cake was washed with 4 L of 3:1 heptanes:THF solution and then with an additional 4 L of the solution as a displacement wash, and the resulting white granular solid dried under nitrogen/vacuum overnight. HPLC assay conditions: same as in Step 1.

### Step 3: Mesylate of CBZ-Proline Amide

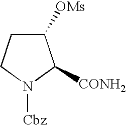

DCM (38.5 L) and CBZ-protected amide (3.85 kg; 14.6 moles; 1.0 eq.) were charged to a 100 L cylindrical flask and the resulting slurry was cooled to −20° C., after which triethylamine (4.1 L; 2 eqs.; d=0.726) was added. MsCl (1.4 L; 1.2 eqs.; d=1.474) was then added over 1 hour, while maintaining the internal temp at −15° C. The reaction was aged for 30 minutes at −15° C. and then assayed for completion using LC. Additional MsCl (113 mL, 1.45 moles) was then added to drive the reaction to completion. After completion, the reaction was quenched with aqueous 3 wt. % NH₄Cl (5 mL per g of SM=starting material; 19.25 L). The layers were partitioned and washed with 3 wt. % NH₄Cl (5 mL/g SM; 19.25 L; 5 eqs.) and then with 10% brine (5 mL/g SM; 19.25 L). The final organic layer was then slowly passed thru a filter frit containing MgSO₄ (1 g/g of product). The KF of the organic solution after MgSO₄ treatment was 2149 μg/mL. The MgSO₄ cake was washed with DCM (2×1 mL/g). The combined filtrate and washes had KF=1896 μg/mL and a concentration=126.2 mg/g of solution, which was concentrated to 140 mg/g of solution; 178 mg/mL; KF=1555 μg/mL.

### Step 4: Sulfonate Salt of CBZ-Proline Amide Mesylate

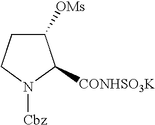

2-Picoline (2 L; 6.1 eq.; d=0.944) was charged to a 100 L cylindrical vessel containing a solution of CBZ proline amide mesylate (5 kg; 14.6 moles; 1.0 eq.) in DCM (141 mg mesylate/g of solution; 25 L) initially cooled to −20 C, wherein the internal temperature is maintained at a maximum of −7° C. during the charging. ClSO₃H (3.5 L; 3.61 eqs.; d=1.745) was then added slowly over 1 hour while maintaining the internal temp at <−15° C. Upon completion of the addition the reaction mixture was aged at 40° C. for about 6 hours, after which the mixture was cooled to room temperature. The reaction mixture was then quenched by adding the mixture over 1.5 hours to a 100 L flask equipped with an overhead strirrer and containing 0.5 M K₂HPO₄ (50 L) cooled to 0° C. After completion of the reverse quench, the resulting slurry was aged for 1 hour at 0° C. The solids were then filtered and the mother liquor was recycled to wash out the crystalline solids still in the reaction flask. The solids were then slurry washed with cold 0.5 M K₂HPO₄ (2×5 mL/g of product=2×25 L), displacement washed with cold IPA (5 mL/g of product; 25 L), and then slurry washed with cold IPA (5 mL/g of product; 25 L).

### Step 5: Lactam Sulfonate

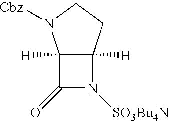

DMF (15 L) was charged to a 50 L round bottom flask, followed by KHCO₃ (978 g; 9.77 moles; 1.5 eqs.) and water (1.5 L). The resulting slurry was heated to 80° C. with a steam pot. The progress of the reaction was monitored by LC. No starting material was observed after 2 hours at 80° C., and the reaction mixture was then cooled with an ice bath to 20° C.

Water (45 L), Bu₄NHSO₄ (2.21 kg; 6.51 moles; 1.0 eq.) and KHCO₃ (651 g, 6.51 moles; 1.0 eq.) were charged to a separate 100 L cylindrical vessel. After CO₂ evolution was complete, pH of the solution was about 7. DCM (15 L) was then charged while stirring the solution. The crude reaction mixture in the round bottom flask was transferred under nitrogen (via a Yamada pump) to the cylindrical vessel and the round bottom was rinsed with water (7.5 L). The mixture was stirred vigorously for 10 minutes after which the biphasic solution was separated. The organic cut was concentrated on a rotary evaporator to approximately 9 L, then solvent switched into IPA (20 L used in the switch). The solution was then cooled to and stored overnight at 5° C. Wt. of product as determined by LC=8.6 kg, KF=850 ppm.

### Step 6: Deprotected Lactam Sulfonate

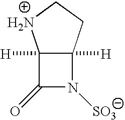

The IPA solution of lactam sulfonate (8.6 kg; 6.51 moles; 1.0 eq.) prepared in Step 5 was charged to a 10 gallon autoclave, followed by the charging of Pd(OH)₂/C catalyst (225 g; 9.77 moles; 1.5 eqs.) slurried in IPA. Hydrogenation was performed at 40 psig which resulted in a mildly exothermic reaction (i.e., temperature increased from an 16° C. to 28° C.) that was complete after about 1.75 hours according to LC. The batch was transferred to a polyjug and the vessel was rinsed with IPA (10 L). The catalyst was filtered over celite and the celite cake washed with IPA. The resulting solution (20.9 kg) was stored overnight at 5° C. The next day, the solution was charged under vacuum through a 1 micron filter into a 75 L round bottom flask and the storage container being rinsed with IPA (300 mL). In a separate flask, TsOH—H₂O (980 g; 5.15 moles) was dissolved in IPA (4 L), and the TsOH/IPA solution was then charged to the product solution via an addition funnel over 2 hours to provide a slurry. During the acid addition a mild exotherm was observed (from 12° C. to 17.3° C.) and the pH changed from 10 to 5. The slurry was filtered and the solids were washed with IPA and dried under nitrogen overnight.

### Step 7: Homopiperidine Salt

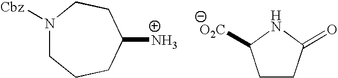

A solution of benzyl (4S)-4-aminoazepane-1-carboxylate (7 kg; 15.78 moles; 1.0 eq.) (Note—can be prepared in accordance with Step 1 of Preparative Example 3, but see also preparative step 7a below) was charged under vacuum to a 75 L round bottom flask. Ethanol (11 kg) was then added to the flask and the mixture was heated to 55° C., after which pyroglutamic acid (2.1 kg; 16.3 moles; 1.03 eqs.) was charged portionwise resulting in a slight exotherm to 60° C. The mixture was aged for 20 minutes and then EtOAc (3.3 L) was added over 10 minutes. The yellow solution was seeded with 70 g of the amine salt product from a previous crystallization run (Note—the procedure does not require seed) and the resulting slurry aged for 1 hour. Additional EtOAc (19.2 L) was then added to the thickened slurry over 2 hours, and then the slurry was cooled to room temperature and aged overnight. The slurry was then filtered in a filter pot and the solids washed with EtOH/EtOAc (1:2) followed by 100% EtOAc to provide the title compound.

### Step 7a Preparation of benzyl (4S)-4-aminoazepane-1-carboxylate

Step 7a-i: Benzyl 4-ethoxycarbonyl-5-oxo-azepane-1-carboxylate

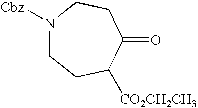

To a solution of Cbz-piperidone (214,6 g; 0.92 mole; 1 eq.) in MTBE (1.4 L) at 0 C was charged BF₃—OEt₂ (118 mL; 0.93 mole; 1.01 eqs/; d=1.12) and the solution was cooled to −30° C. Ethyl diazoacetate (N₂CH₂CO₂Et) (136.9 g; 1.2 moles; 1.3 eqs.) was added slowly at a rate such that the internal temperature of the reaction mixture was between −27° C. to −30° C. The addition period was two hours and N₂ evolution was observed during this period of time.

After the addition was complete, the solution was warmed to 0° C. and the reaction was assayed by HPLC to be complete. Aqueous K₂CO₃ (4.4 wt % solution in water; 1.4 kg, 0.45 mole; 0.49 eq.) was then added to the reaction mixture at 0° C. and the solution was warmed to 20° C. The biphasic mixture was separated and the organic solution was washed with water (0.5 L) followed by aqueous KHCO₃ (23.1 wt % aqueous solution, 163 g of solution; 0.375 mole; 0.41 eq.). The organic solution was concentrated under vacuum at 50° C. to an oil. Toluene (250 mL) was then added and the solution was concentrated again under vacuum at 90° C. until most of the toluene was removed and an oil remained. The product was cooled to 20° C. and stored for subsequent use.

### Step 7a-ii: Potassium Enolate

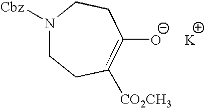

To a solution of benzyl 4-ethoxycarbonyl-5-oxo-azepane-1-carboxylate (293.6 g; 0.92 mole; 1.0 eq.) in MeOH (800 mL) was added KOMe (147.3 g; 2.1 moles; 2.3 eqs.) and the slurry was heated to 65° C. The slurry was aged at 65° C. for 30 minutes and then cooled to 50° C. over 20 minutes. After the slurry had cooled to 50° C., MTBE (800 mL) was added over 20 minutes and the slurry was cooled slowly to 0° C. over 2 hours. The slurry was filtered and the filter cake (potassium enolate product) was washed with 1:1 MeOH:MTBE (400 mL) followed by MTBE (400 mL). The solids were dried under a stream of nitrogen.

### Step 7a-iii: N-CBZ-4-azepenone

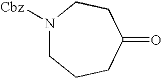

A slurry of K-enolate (264 g; 0.77 mole; 1.0 eq.), MeOH (600 mL) and water (200 mL) was heated to 65° C. and aged until most of the solids dissolved. Aqueous KOH (45 wt %; 12.47 g; 0.1 mole) was added and the solution was heated for another hour between 65° C. and 70° C. A portion of the solution (300 mL) was then distilled off under vacuum and MTBE (600 mL) and water (900 mL) were added. The solution was cooled to room temperature and the organic and aqueous phases were separated. The organic solution was washed with NaCl (1.5 wt %, 1 L) and then concentrated to an oil under vacuum at 65° C. The residue was cooled to 20° C. and stored at room temperature for subsequent use.

### Step 7a-iv: N—CBZ-4R-azepanol

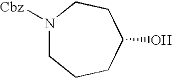

N—CBZ-4-azepenone (20.02 g; 0.081 mole; 1.0 eq.) in DMF (40 mL) was heated to 30° C. in a reaction vessel, after which a solution of NADP (400 mg) and PDH-101 (226.8 mg) in 10 mL of a 0.3 M Na₂(PHO₃) pH 7.0 buffer and a solution of KRED-112 (225.4 mg) in 10 mL of the 0.3 M Na₂(PHO₃) pH 7.0 buffer were added. The reaction mixture was aged at 30° C. for 16-18 hours. Upon completion, Celite 521 (10.46 g) and NaCl (80.42 g) were added and the mixture heated to about 90° C. for about thirty minutes. After cooling to below 65° C., EtOAc (120 mL) was added and the mixture was filtered through Celite. The filter cake was washed with EtOAc (160 mL), the filtrate transferred to an extraction funnel and the phases separated. The filter cake was washed again with EtOAc (90 mL). The combined organic layers were washed with brine (30 mL) and concentrated. 94% EE by SFC.

### Step 7a-v: N—CBZ-4R-mesyloxyazepane

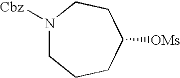

To a solution of the azepanol (4.587 kg; 18.4 moles; 1.0 eq.) and Et₃N (3.08 L; 22.1 moles; 1.2 eqs.) in EtOAc (27.6 L) at −10° C. to −15° C. was charged MsCl (1.5 L; 19.3 moles; 1.05 eqs.) over 1-2 hours. After the addition was complete, the solution was warmed to 0° C. and aged for 1 hour. Aqueous NaHSO₄ was added to the reaction mixture, and the solution was warmed to room temperature. The organic/aqueous phases were separated and the organic solution was washed with aqueous Na₂SO₄. The organic solution was concentrated to an oil under vacuum at 40-45° C.

### Step 7a-vi: N—CBZ-4S-azidoazepane

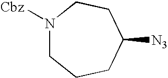

To a solution of mesylate (6.024 kg; 18.4 moles; 1.0 eq.) in DMF (9.2 L) was charged Na₂CO₃ (98 g; 0.92 moles; 0.05 eq.) and n-Bu₄NHSO₄ (62 g; 0.18 mole; 0.01 eq.) and the mixture was heated to 40-50 C for 20 minutes under nitrogen. NaN₃ (2.21 kg; 36.8 kg; 2.0 eqs.) was then added and the solution was stirred for an additional 20 minutes at 40-50° C. The solution was then heated further to 75-80° C. and aged for 1-2 hours. After the aging period, the solution was cooled to 50° C. and MTBE (27.6 L) was charged. The solution was allowed to cool to 30° C. and water (36.8 L) was added. The phases were separated and the aqueous solution was extracted with MTBE (5.5 L). The combined organic solutions were washed with water (2×36.8 L each time) and then concentrated to an oil under vacuum at 40-45° C. EtOH (3.7 L) was charged to the residual oil and the solution was concentrated under vacuum (40-45° C.) again to give an oil.

### Step 7a-vii: Benzyl (4S)-4-aminoazepane-1-carboxylate

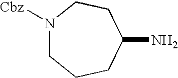

To a solution of the azide (5.047 kg; 18.4 moles; 1.0 eq.) in EtOH (3.7 L) at 80° C. was charged P(OEt)3 (3.669 kg; 22.1 moles; 1.2 eqs.) Nitrogen gas evolution was observed. Water (˜1.5 L) was charged to the reaction mixture over 10-30 minutes at 90° C. followed by additional water (˜4.6 L) charged over 5 minutes. The solution was aged for 10 minutes at 80° C. and aqueous 5 N HCl (7.36 L) was added over 10 minutes. The solution was then aged at 80° C. for 1 hour. Solvent (about 3.7 L) was then removed by vacuum distillation at 80° C. The reaction was cooled to 50° C. and the reaction solution washed with IPAc (27.6 L). The phases were separated, the IPAc cut was washed with water (3.7 L), and NaOH (5 M; 14.7 L) slowly added to the combined aqueous cuts. A vacuum was applied to the solution (distilling off approximately 0.1-0.2 L/mol, 2-4 L) such that the reaction temperature was <40° C. during the NaOH charge. The aqueous solution was then extracted twice with CH₂Cl₂ (14.7 L, then 7.3 L) and the combined organic solutions were dried over K₂CO₃, filtered, and concentrated under vacuum to an oil.

### Step 8: Amine Activation

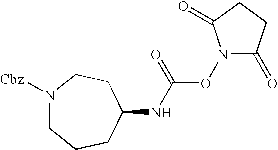

ACN (10 vol, 28.6 L) was charged to the 100 L extractor and cooled to below 10° C., after which amine pyroglutamate (2.86 kg; 7.6 moles; 1.0 eq.) was charged followed by Et₃N (1.15 L; 8.3 moles; 1.1 eqs.) at a temperature of 5° C. The cold white slurry was further cooled to 2.5° C. Succinyl carbonate (2.13 kg; 8.3 moles; 1.1 eqs.) was added to the slurry over a period of 20 minutes, during which time the slurry slowly thinned and eventually cleared. The solution was allowed to age for 30 minutes at 2° C. Upon completion of the ageing the reaction mixture was assayed and was determined via LC to have undergone 100% conversion.

EtOAc (28.6 L) was added to the cold solution followed by water (14.3 L). The solution was stirred for 5 minutes and then allowed to settle. 15% NaCl solution (1 L) was added to the solution to help cut the layers. The aqueous layer was removed and 5% NaCl solution (14.3 L) was pumped into the extractor. The solutions were again stirred for 5 minutes and then allowed to settle. The aqueous solution was removed and the organic layer was collected and dried over MgSO₄ (100 wt %, 3 kg).

The dried organic solution was slowly filtered through a 1μm inline filter into a 75 L round bottom flask fitted with a batch concentrator. The solution was concentrated to about 9 L and flushed twice in succession with EtOAc (15 L), and then flushed twice more with EtOAc (6.2 L) to provide a solution (about 7 L) with KF=692 ppm. DMF (14.3 L) was then slowly bled into the solution for immediate use in the next step.

### Step 9: Tetrabutyl ammonium salt of (1S,5R)-2-[({(4S)-1-[(benzyloxy)carbonyl]azepan-4-yl}amino)carbonyl]-7-oxo-2,6-diazabicyclo[3.2.0]heptane-6-sulfonic acid

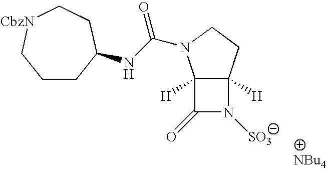

On the first day, the DMF solution of activated amine carbamate (Step 8; 2.95 kg; 7.6 moles; 1 eq.) in a 75 L round bottom flask was cooled below 10° C. via ice bath. Bicyclic lactam (Step 6; 1.38 kg; 0.95 eq.) was charged to the cold solution followed by NaHCO₃. The white slurry was further cooled to 4.1° C. Cooled water (7.2 L) was then slowly charged slowly 15 minutes (Tₘₐₓ=13.8° C.) accompanied by CO₂ evolution. Upon completion of the water addition, the ice bath was removed and the reaction mixture was allowed to warm slowly to room temperature slowly and then aged until the reaction was complete (12 hours) as determined by LC assay for 8 hours. The reaction solution was pumped into a 100 L extractor and cooled to below 10° C. after which cooled water (29.5 L) was slowly added to the solution followed by EtOAc (14.75 L). The solution was stirred for 5 minutes and allowed to settle. The EtOAc layer was removed and an additional 5 vol EtOAc (14.75 L) was added to the aqueous layer. The solution was stirred for 5 minutes, allowed to settle, and the EtOAc removed. The aqueous layer was charged to the 100 L extractor along with DCM (29.5 L). The emulsion was cooled to below 10° C. and then Bu₄NSO₄ (2.3 kg; 1.0 eq.) was added. The mixture was allowed to stir for 30 minutes at 6° C. The layers were allowed to settle and the aqueous layer was removed. The remaining DCM (organic) layer was washed twice with water (2×14.75 L). The organic layer was then collected and dried over MgSO₄ (50 wt %, 1.5 kg), filtered, and stored overnight at 5 C.

The next day the dried organic layer was filtered through a 1 μm inline filter into a 75 L round bottom flask fitted with a batch concentrator. The solution was concentrated to approximately 9 L and flushed with DCM (4 L) and concentrated to 9 L and KF measured (1223 ppm). The DCM solution was concentrated to a volume of about 5 l, and then DMF (14.4 L) was slowly bled into the solution. The solution was then concentrated and stored at 5° C. for use in the next step.

### Step 10: (1S,5R)-2-{[(4S)-Azepan-4-ylamino]carbonyl}-7-oxo-2,6-diazabicyclo-[3.2.0]-heptane-6-sulfonic acid

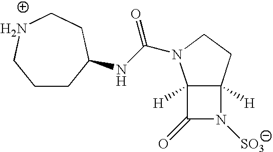

A slurry of 5% Pd/C (104 g) in DMF (6.4 L) was charged to a 10 gallon autoclave, after which hydrogen pressure (40 psig) was applied and the slurry aged for 1 hour. A solution of the tetrabutyl ammonium salt of (1S,5R)-2-[({(4S)-1-[(benzyloxy)carbonyl]azepan-4-yl}amino)carbonyl]-7-oxo-2,6-diazabicyclo[3.2.0]heptane-6-sulfonic acid (3.46 kg; 4.89 moles; 1.0 eq.) in DMF (17.3 L; 8.3 moles) (see Step 8) was degassed for 30 minutes and then charged to the autoclave vessel. Hydrogenation was performed until reaction was complete as indicated by LC (about 40 minutes). The batch was transferred to a polyjug container and the autoclave vessel rinsed with IPA (17.3 L). The product and IPA rinse solutions were transferred to a 100 L cylindrical vessel and mixed thoroughly. After 10 minutes, the slurry was filtered over celite, and the celite cake was washed with IPA (17.3 L). The filtrate was transferred to a 100 L round bottom flask via an in-line filter. MP-TMT resin (1.4 kg) was added to the solution and the slurry was aged for 1 hour, then filtered in a 10-inch filter pot, and the resin washed with IPA (10.4 L). The filtrate was transferred via an in-line filter to a new 100 L round bottom flask and formic acid (553 mL) was charged over the course of 1 hour, generating a free-flowing crystalline slurry. The slurry was aged for 30 minutes, filtered in an 18-inch filter pot, and the solids were rinsed with 1.8 L of DMF:IPA (5:13), followed by IPA (3 L). The solids were dried under vacuum overnight to give a crystalline material which was determined (by, e.g., TGA, DSC, and LCMS) to be an IPA solvate of the title compound.

### Example 34

### Crystalline dihydrate of Compound 1

### Part A: Preparation

Water (3 L) and IPA (12 L) were charged to a 22 L round bottom flask, followed by an IPA solvate of Compound 1 (1.579 kg) prepared in the manner described in Step 10 of Example 33. The resultant slurry was heated to 40° C. and then aged for 20 minutes, after which additional IPA (3 L) was then added over 1 hour. The solution was then allowed to cool to room temperature during the next 2 hours. Additional IPA (6 L) was then added over 2 hours and an assay (HPLC) of the mother liquors at this point was 4.2 mg/g. The slurry was filtered and the resulting crytalline solids were washed with 3 L IPA/water (4:1) and dried overnight under vacuum at room temperature. Isolated solids: 1.361 kg, 89.4 wt %, >99% A % via HPLC. The crystalline product was determined to be a dihydrate using KF titration and TGA and subsequently confirmed via single crystal studies. XRPD, DSC, and TGA characterizations are described in Part B

### Part B: Characterization

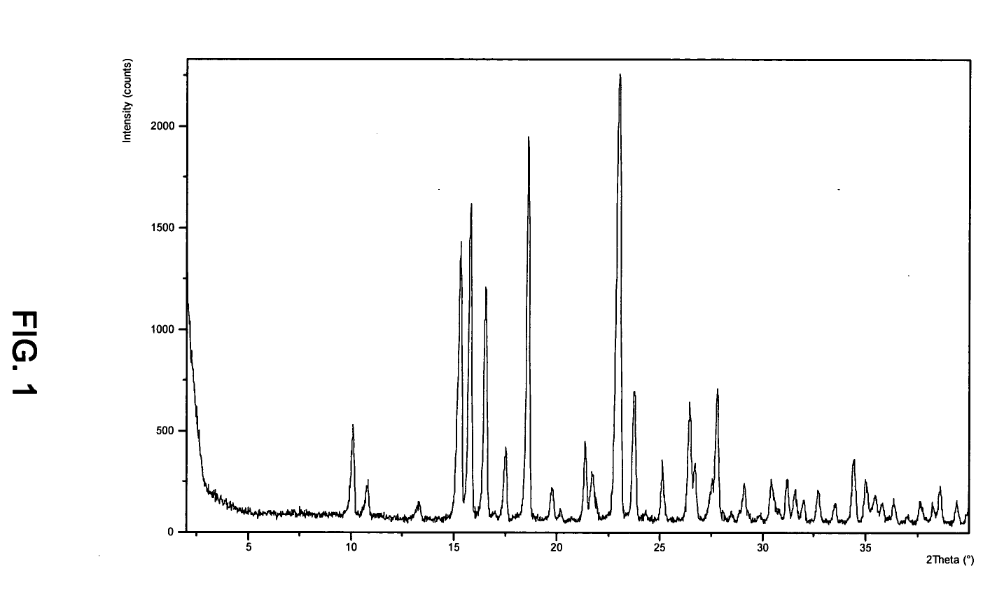

An XRPD pattern of the crystalline dihydrate of Compound 1 prepared in accordance with the method described in Part A was generated on a Philips Pananalytical X'Pert Pro X-ray powder diffractometer with a PW3040/60 console using a continuous scan from 2.5 to 40 degrees 2Θ. Copper K-Alpha 1 (K_{α1}) and K-Alpha 2 (K_{α2}) radiation was used as the source (i.e., PW3373/00 ceramic Cu LEF X-ray tube K-Alpha radiation source). The experiment was conducted with the sample at room temperature and open to the atmosphere. The XRPD pattern is shown in FIG. 1. 2Θ values, the corresponding d-spacings, and the relative peak intensities in the XRPD pattern include the following:

TABLE
XRPD of crystalline dihydrate

| Peak No. | d-spacing (Å) | 2 Theta | I/Imax (%)¹ |
|---|---|---|---|
| 1 | 8.75 | 10.1 | 0.17 |
| 2 | 8.16 | 10.8 | 0.03 |
| 3 | 6.64 | 13.3 | 0.04 |
| 4 | 5.77 | 15.3 | 0.49 |
| 5 | 5.59 | 15.8 | 0.62 |
| 6 | 5.35 | 16.6 | 0.39 |
| 7 | 5.21 | 17.0 | 0.01 |
| 8 | 5.05 | 17.5 | 0.06 |
| 9 | 4.77 | 18.6 | 0.58 |
| 10 | 4.49 | 19.8 | 0.04 |
| 11 | 4.39 | 20.2 | 0.01 |
| 12 | 4.15 | 21.4 | 0.11 |
| 13 | 4.09 | 21.7 | 0.09 |
| 14 | 3.86 | 23.0 | 1.00 |
| 15 | 3.75 | 23.7 | 0.32 |
| 16 | 3.66 | 24.3 | 0.01 |
| 17 | 3.54 | 25.1 | 0.05 |
| 18 | 3.37 | 26.5 | 0.18 |
| 19 | 3.34 | 26.7 | 0.07 |
| 20 | 3.240 | 27.5 | 0.07 |
| 21 | 3.206 | 27.8 | 0.21 |
| 22 | 3.129 | 28.5 | 0.01 |
| 23 | 3.068 | 29.1 | 0.03 |
| 24 | 2.989 | 29.9 | 0.01 |

¹The relative intensities of the XRPD peaks are a strong function of the preferred orientation of the sample, and thus can vary significantly from sample to sample due to differences in sample preparation (e.g., degree of grinding).

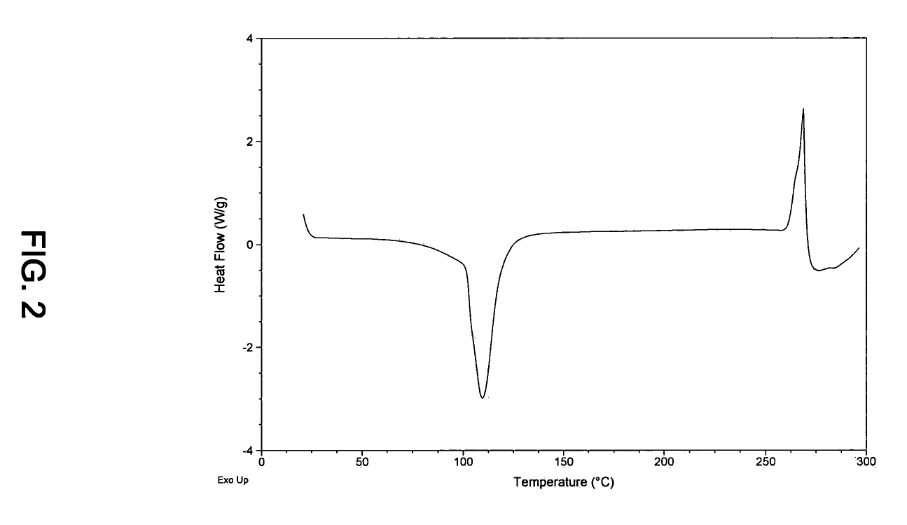

Crystalline dihydrate of Compound 1 prepared in accordance with the method described in Part A was also analyzed with a TA Instruments DSC 2910 differential scanning calorimeter (DSC) at a heating rate of 10° C./minute from 25° C. to 300° C. in a crimped (i.e., closed) aluminum pan. The data were analyzed using the DSC analysis program contained in the system software. The DSC curve (see FIG. 2) exhibited an endotherm with an onset temperature of 100.8° C. and a peak temperature of 109.6° C. The enthalpy change was 275.6 J/g. The endotherm is believed to be due to dehydration.

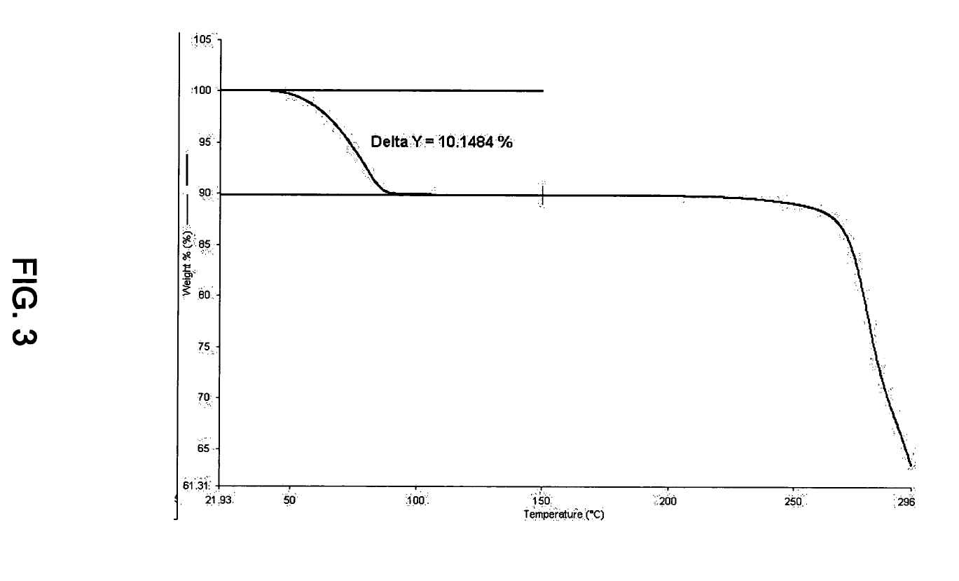

A thermogravimetric analysis (TGA) of crystalline dihydrate of Compound 1 prepared in accordance with the method described in Part A was performed with a Perkin Elmer model TGA 7 under a flow of nitrogen at a heating rate of 10° C./minute from 20° C. to 300° C. Analysis of the results was carried out using the Delta Y function within the instrument software. A weight loss of 10.1% associated with loss of water up to 98.3° C. was observed. The TGA curve is shown in FIG. 3.

While the foregoing specification teaches the principles of the present invention, with examples provided for the purpose of illustration, the practice of the invention encompasses all of the usual variations, adaptations and/or modifications that come within the scope of the following claims.

1. A compound of Formula I: 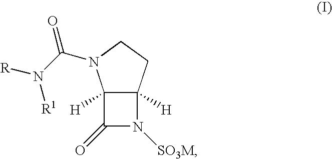
or a pro-drug or pharmaceutically acceptable salt thereof, wherein:
R represents a 7-, 8-, or 9-membered saturated or unsaturated ring optionally containing from 1 to 3 heteroatoms independently selected from N, O and S, wherein the ring is optionally substituted with one or more R^{a} groups;
R¹ represents hydrogen or methyl;
each R^{a} independently represents hydrogen, C₁₋₆ alkyl, halo, —(CH₂)ₙCN, —(CH₂)ₙNO₂, —(CH₂)ₙOR^{b}, —(CH₂)ₙSR^{b}, —(CH₂)ₙN(R^{b})₂, —(CH₂)ₙC(O)N(R^{b})₂, —(CH₂)ₙSO₂N(R^{b})₂, —(CH₂)ₙCO₂R^{b}, —(CH₂)ₙC(O)R^{b}, —(CH₂)ₙOC(O)R^{b}, —(CH₂)ₙNHC(O)R^{b}, —(CH₂)ₙNHC(O)₂R^{b}, —(CH₂)ₙNHSO₂R^{b}, —(CH₂)ₙC(═NH)NH₂, or —(CH₂)ₙC(═NH)H; or two R^{a} groups on the same ring carbon atom are optionally taken together to form oxo; or two R^{a} groups on the same ring sulfur atom are optionally taken together with the sulfur to represent SO; or four R^{a} groups on the same ring sulfur atom are optionally taken together with the sulfur to represent SO₂;
each n is independently 0, 1, 2, 3, or 4;
each R^{b} independently represents hydrogen or C₁₋₄ alkyl; and
M represents hydrogen or a pharmaceutically acceptable cation or, when the compound contains an internal base which is capable of being protonated by a sulfonic acid, M is optionally a negative charge.

2. The compound according to claim 1, or a prodrug or pharmaceutically acceptable salt thereof, wherein R¹ is hydrogen.

3. The compound according to claim 1, or a prodrug or pharmaceutically acceptable salt thereof, wherein R is a 7-, 8-, or 9-membered saturated ring containing one nitrogen atom and a balance of carbon atoms.

4. The compound according to claim 3, or a prodrug or pharmaceutically acceptable salt thereof, wherein R¹ is hydrogen.

5. The compound according to claim 1, or a prodrug or pharmaceutically acceptable salt thereof, wherein:
R is an 7-, 8- or 9-membered saturated ring containing N(R^{a}) and optionally also containing either O or NH; wherein the two ring atoms adjacent and directly bonded to N(R^{a}) are carbon atoms and (i) one of the ring carbons directly bonded to the N(R^{a}) is optionally substituted with oxo or is optionally mono-substituted with methyl or is optionally di-substituted with methyl, or (ii) both of the ring carbons directly bonded to the N(R^{a}) are independently and optionally mono- or di-substituted with methyl;
R¹ is hydrogen; and
R^{a} is hydrogen, C₁₋₄ alkyl, —(CH₂)₂₋₃OH, —(CH₂)₂₋₃O—C₁₋₃ alkyl, —(CH₂)₂₋₃NH₂, —(CH₂)₂₋₃N(H)—C₁₋₃ alkyl, —(CH₂)₂N(—C₁₋₃ alkyl)₂, —C(NH)NH₂, or —C(═NH)H.

6. The compound according to claim 5, or a prodrug or pharmaceutically acceptable salt thereof, wherein:
R is an 7-, 8- or 9-membered saturated ring containing N(R^{a}), wherein R^{a} is hydrogen, CH₃, —(CH₂)₂OH, —(CH₂)₂NH₂, —(CH₂)₂N(H)CH₃, or —(CH₂)₂N(CH₃)₂.

7. The compound according to claim 1, or a pharmaceutically acceptable salt thereof, which is a compound represented by Formula II or III:

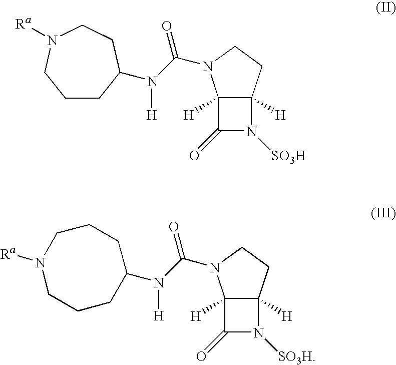

8. The compound according to claim 7, or a pharmaceutically acceptable salt thereof, wherein R^{a} is hydrogen, C₁₋₆ alkyl, —C(═NH)NH₂, or —C(═NH)H.

9. The compound according to claim 8, or a pharmaceutically acceptable salt thereof, wherein R^{a} is H or C₁₋₄ alkyl.

10. The compound according to claim 1, or a pharmaceutically acceptable salt thereof, wherein R is: 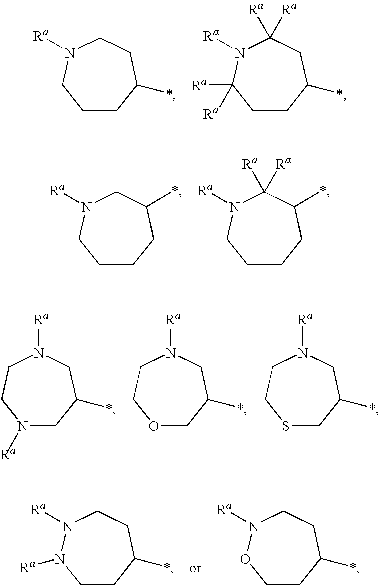
wherein the asterisk (*) at the end of the bond denotes the point of attachment of R to the rest of the compound;
R¹ is H;
each R^{a} which is a substituent on a ring N is independently selected from the group consisting of H, CH₃, —(CH₂)₂₋₃OH, —(CH₂)₂NH₂, —(CH₂)₂N(H)CH₃, —(CH₂)₂N(CH₃)₂. —(CH₂)₁₋₂C(O)NH₂, —(CH₂)₁₋₂C(O)N(H)CH₃, —(CH₂)₁₋₂C(O)N(CH₃)₂,
each R^{a} which is a substituent on a ring carbonn is independently H or CH₃ or, in the event that two R^{a} groups are on the same ring carbon atom, the two R^{a} groups are optionally taken together to form oxo; with the proviso that at least one R^{a} on a ring carbon is other than H; and
M is H.

11. The compound according to claim 1, or a pharmaceutically acceptable salt thereof, which is selected from the group consisting of:
(1S,5R)-2-{[(4S)-azepan-4-ylamino]carbonyl}-7-oxo-2,6-diazabicyclo[3.2.0]heptane-6-sulfonic acid;
(1S,5R)-2-{[(4R)-azepan-4-ylamino]carbonyl}-7-oxo-2,6-diazabicyclo[3.2.0]heptane-6-sulfonic acid;
(1S,5R)-2-{[(cycloheptylamino]carbonyl}-7-oxo-2,6-diazabicyclo-[3.2.0]-heptane-6-sulfonic acid;
(1S,5R)-2-{[(3 S)-azepan-3-ylamino]carbonyl}-7-oxo-2,6-diazabicyclo[3.2.0]heptane-6-sulfonic acid;
(1S,5R)-7-oxo-2-({[(3 S)-2-oxoazepan-3-yl]amino}carbonyl)-2,6-diazabicyclo[3.2.0]heptane-6-sulfonic acid;
(1S,5R)-2-[(1,4-diazepan-6-ylamino)carbonyl]-7-oxo-2,6-diazabicyclo[3.2.0]heptane-6-sulfonic acid;
(1S,5R)-2-{[(6R)-1,4-oxazepan-6-ylamino]carbonyl}-7-oxo-2,6-diazabicyclo[3.2.0]heptane-6-sulfonic acid;
(1S,5R)-2-{[(6S)-1,4-oxazepan-6-ylamino]carbonyl}-7-oxo-2,6-diazabicyclo[3.2.0]heptane-6-sulfonic acid;
(1S,5R)-2-({[(4S)-1-methylazepan-4-yl]amino}carbonyl)-7-oxo-2,6-diazabicyclo[3.2.0]heptane-6-sulfonic acid;
(1S,5R)-2-({[(4S)-1-(2-hydroxyethyl)azepan-4-yl]amino }carbonyl)-7-oxo-2,6-diazabicyclo[3.2.0]heptane-6-sulfonic acid;
(1S,5R)-2-({[(4S)-1-(3-hydroxypropyl)azepan-4-yl]amino}carbonyl)-7-oxo-2,6-diazabicyclo-[3.2.0]heptane-6-sulfonic acid;
(1S,5R)-2-[({(4S)-1-[2-(amino)ethyl]azepan-4-yl}amino)carbonyl]-7-oxo-2,6-diazabicyclo[3.2.0]heptane-6-sulfonic acid;
(1S,5R)-2-[({(4S)-1-[2-(dimethylamino)ethyl]azepan-4-yl}amino)carbonyl]-7-oxo-2,6-diazabicyclo[3.2.0]heptane-6-sulfonic acid;
(1S ,5R)-7-oxo-2-{[(2,2,7,7-tetramethylazepan-4-yl)amino]carbonyl}-2,6-diazabicyclo-[3.2.0]-heptane-6-sulfonic acid;
(1S,5R)-2-[(azocan-5-ylamino)carbonyl]-7-oxo-2,6-diazabicyclo-[3.2.0]-heptane-6-sulfonic acid;
(1S,5R)-2-[(azocan-4-ylamino)carbonyl]-7-oxo-2,6-diazabicyclo[3.2.0]heptane-6-sulfonic acid (Isomer A);
(1S,5R)-2-[(azocan-4-ylamino)carbonyl]-7-oxo-2,6-diazabicyclo[3.2.0]heptane-6-sulfonic acid (Isomer B);
(1S,5R)-2-{[azonan-5-ylamino]carbonyl}-7-oxo-2,6-diazabicyclo[3.2.0]heptane-6-sulfonic acid (Isomer A)
(1S,5R)-2-{[azonan-5-ylamino]carbonyl}-7-oxo-2,6-diazabicyclo[3.2.0]heptane-6-sulfonic acid (Isomer B)
(1S,5R)-7-oxo-2-{[(6R)-1,4-thiazepan-6-ylamino]carbonyl}-2,6-diazabicyclo[3.2.0]heptane-6-sulfonic acid;
(1S,5R)-2-({[(4S)-1-(2-amino-2-oxoethyl)azepan-4-yl]amino}carbonyl)-7-oxo-2,6-diazabicyclo[3.2.0]heptane-6-sulfonic acid;
(1S,5R)-2-({[(4S)-1-(iminomethyl)azepan-4-yl]amino}carbonyl)-7-oxo-2,6-diazabicyclo[3.2.0]heptane-6-sulfonic acid;
(1S,5R)-7-oxo-2-({[(4S)-7-oxoazepan-4-yl]amino}carbonyl)-2,6-diazabicyclo[3.2.0]heptane-6-sulfonic acid;
(1S,5R)-7-oxo-2-({[(4R)-7-oxoazepan-4-yl] amino }carbonyl)-2,6-diazabicyclo[3.2.0]heptane-6-sulfonic acid;
(1S,5R)-2-[(1,2-diazepan-5-ylamino)carbonyl]-7-oxo-2,6-diazabicyclo[3.2.0]heptane-6-sulfonic acid;
(1S,5R)-2-{[(5R)-1,2-oxazepan-5-ylamino]carbonyl}-7-oxo-2,6-diazabicyclo[3.2.0]heptane-6-sulfonic acid;
(1S,5R)-2-({[4-(3-aminopropyl)cycloheptyl]amino }carbonyl)-7-oxo-2,6-diazabicyclo[3.2.0]heptane-6-sulfonic acid;
(1S,5R)-2-({[(1S,4R)-4-aminocycloheptyl]amino}carbonyl)-7-oxo-2,6-diazabicyclo[3.2.0]heptane-6-sulfonic acid;
(1S,5R)-2-[(1,5-diazocan-3-ylamino)carbonyl]-7-oxo-2,6-diazabicyclo[3.2.0]heptane-6-sulfonic acid;
(1S,5R)-2-{[(7R)-1,4-oxazocan-7-ylamino]carbonyl}-7-oxo-2,6-diazabicyclo[3.2.0]heptane-6-sulfonic acid; and
(1S ,5R)-7-oxo-2-{[(4R)-2,3,4,7-tetrahydro-1H-azepin-4-ylamino]carbonyl}-2,6-diazabicyclo[3.2.0]heptane-6-sulfonic acid.

12. The compound according to claim 11, which is (1S,5R)-2-{[(4S)-azepan-4-ylamino]carbonyl}-7-oxo-2,6-diazabicyclo-[3.2.0]-heptane-6-sulfonic acid or a pharmaceutically acceptable salt thereof.

13. A pharmaceutical composition which comprises a compound according to claim 1, or a pharmaceutically acceptable salt thereof, and a pharmaceutically acceptable carrier.

14. The pharmaceutical composition according to claim 13, which further comprises a beta-lactam antibiotic and a DHP inhibitor.

15. A combination of a beta-lactam antibiotic and a compound according to claim 1, or a pharmaceutically acceptable salt thereof.

16. A combination of a beta-lactam antibiotic, a DHP inhibitor, and a compound according to claim 1, or a pharmaceutically acceptable salt thereof.

17. (canceled)

18. (canceled)

19. (canceled)

20. A method for treating a bacterial infection which comprises administering to a subject in need of such treatment a therapeutically effective amount of a compound according to claim 1, or a prodrug or pharmaceutically acceptable salt thereof, optionally in combination with a beta-lactam antibiotic.

21. (canceled)

22. The compound according to claim 12, which is in the form of a crytalline dihydrate characterized by an X-ray powder diffraction pattern obtained using copper K_{α} radiation which comprises 2Θ values in degrees of about 10.1, 10.8 and 15.3.

23. A process for preparing a crystalline dihydrate according to claim 22, which comprises:
(A) adding a C₁₋₄ alkyl alcohol solvate of the compound according to claim 12 to a mixture comprising water and C₁₋₄ alkyl alcohol to provide a slurry;
(B) ageing the slurry of Step A, optionally with the addition of more C₁₋₄ alkyl alcohol to the slurry during the ageing; and
(C) isolating the crystalline dihydrate from the slurry.

24. The process according to claim 23, wherein the solvate is an IPA solvate and the alcohol employed in the slurry is IPA.
